SEQUENCE LISTING (72 sequences)

SEQ ID NO: 1 - DNA, 3175 bp, Mus musculus
  source 1..3175: mol_type = other DNA
gcctttcctggccgttccctccttctggccgaggtgcctgcgtttaggggtgtcaccctggctcccgggacgccgcctccggagatttaagcgagaactggagtaggtcgtgtacttggagcggacgaggaagccaagagctcggacagaggcggagaggggcgggaagcgcaacaggtcacctggaggaagccccatactgacctcctcatgctgctgacacaggcaggatggcattgaactcagggtcacctccaggaatcggaccttgctatgagaaccacgggtatcagtctgagcacatctgtcctccgagaccaccagtggctcccaatggctacaacttgtatccagcccagtactacccatctccagtgcctcagtatgctccgaggattacaacgcaagcctcaacatctgtcatccacacacatcccaagtcctcaggagcactgtgcacctcaaagtctaagaaatcgctgtgtttagccctcgccctgggcactgtcctcacgggagctgctgtggctgctgtcttgctttggaggttctgggacagcaactgttctacgtctgagatggagtgtgggtcttcaggcacatgcatcagctcttctctctggtgtgacggggtagcacattgtcccaacggagaagatgagaaccgttgtgttcgtctctacggacaaagcttcatcctccaggtttactcatctcagaggaaagcctggtatcccgtgtgccaggatgattggagtgagagctacgggagagcagcatgtaaagacatgggatacaagaacaatttttattctagccaagggataccagaccagagcggggcaacgagctttatgaagctgaatgtgagctcaggcaacgttgacctctataaaaaactctaccacagtgactcatgttcatcccgcatggtggtttctttgcgctgtatagaatgcggggttcgctcagtgaaacgccagagcaggattgtgggtggattgaatgcctcaccaggagactggccctggcaggtcagcctgcacgtccaaggcgtccacgtctgcggaggctccatcatcacccccgagtggattgtgacggccgcccactgtgtggaagaacccctcagcagcccgaggtactggacggcatttgcgggaattctgagacagtctctcatgttctatggaagtagacaccaggtagaaaaagtaatttcccatccaaattacgactctaagaccaagaataacgacattgctctcatgaagctgcagacacctttggcttttaatgatctagtgaagccagtgtgtctgccgaacccaggcatgatgctagacctagaccaggaatgctggatttcggggtggggggccacctatgagaaagggaagacctcggacgtgttgaatgctgccatggtacccttgatcgagccctccaaatgtaatagtaaatacatatacaacaacctaatcacaccagccatgatctgtgccggcttcctccaggggtctgtcgactcttgccagggagacagtggagggccgctggttactttgaagaatgggatctggtggctgattggggacacgagctggggctcgggctgtgccaaggcactcagacctggagtatacgggaacgtgacggtatttacagattggatctaccagcaaatgagggcgaacagctaatccacgtggctttgtcccagacttcctttgtcttcaacaaccttctgcaagaaaaccaagggcctgaattttaacttcctgtgcacaatgtaccttttgagatgattcgaagggcctttcacttttattaaacagtgacttgtttgactgtgctccctggtcctgtgagggcttcagtgccccacccctgggccacttctgcagctcccaccagaatggatgaccagattctgttgggtttgggcacatagggccaaaggcagaggagggtggcactctcatgttggaacttcttttgggctcatgctcaggccttttttggatcactaaggactatgacctctgagtaacctgatgacctgagaaagagtaaggaggccaggcagggccttgggcccaggaacaggtaccttgagagtgagagctacccattgcctgtggcctaaatctgctgtgcaggttgggctggtcatactgtcatgatttcattaacagcctgggtgaacatggctgggagtaaagggcttgctctcctgcatgttgacatgacggccctttccaagggtgatggaggctttcccaagctaagggcctaggcagatctctcagagcaagaagctaatgccggcatgtcccttgggtgagctctacatggtgttattcagtctggttcttggctccccactactgtttctctcagcctctcagagcctgaaacttacctcttagctttggctacaggcatggcctagtacctgatggagcctgtatagctcagctaatcaaatggaggctcaggtccatcagaatcagggacttgtgatttcagtcaccttgcttctgggttgtgtttcttctcttactacctcactgcacctggacactagagtggatgaatgtctggagttcacctgcatttggactgtgtgattgtgcctcagacactagacctcttccagatggttaggttgttctgtagactggcaatgagattagaagttcctagcttcagataaagatgaaagagaggagatcattgtcttctgtcttcttctggccctgggtttataccaggaaagccatgccagaattaccaaatatgaagtatgaatgtcttacccacggtgaggctctgcctccttctctctgcctggttcttcagaaggcagtgaatgggtcataactgggactccatctttgctggggaaagtctcccacctagggaatggttaccactccatgtaaagaaaactccctcatgcgtcctctgggaccttcttagatgctgtaaggtacctacatacagactaaatgtgcaagcaccttgaagtgtgagaacctgtcccctccttagctctccttgtctttgctgttggttggttatttcctgctttgtgtctgttctgagctgtgagattccactgtgaaatatatgaataaagtatataattcttttaaaaaaaaaaaaaaaa

SEQ ID NO: 2 - protein, 490 aa, Mus musculus
MALNSGSPPGIGPCYENHGYQSEHICPPRPPVAPNGYNLYPAQYYPSPVPQYAPRITTQASTSVIHTHPKSSGALCTSKSKKSLCLALALGTVLTGAAVAAVLLWRFWDSNCSTSEMECGSSGTCISSSLWCDGVAHCPNGEDENRCVRLYGQSFILQVYSSQRKAWYPVCQDDWSESYGRAACKDMGYKNNFYSSQGIPDQSGATSFMKLNVSSGNVDLYKKLYHSDSCSSRMVVSLRCIECGVRSVKRQSRIVGGLNASPGDWPWQVSLHVQGVHVCGGSIITPEWIVTAAHCVEEPLSSPRYWTAFAGILRQSLMFYGSRHQVEKVISHPNYDSKTKNNDIALMKLQTPLAFNDLVKPVCLPNPGMMLDLDQECWISGWGATYEKGKTSDVLNAAMVPLIEPSKCNSKYIYNNLITPAMICAGFLQGSVDSCQGDSGGPLVTLKNGIWWLIGDTSWGSGCAKALRPGVYGNVTVFTDWIYQQMRANS

SEQ ID NO: 3 - DNA, 3212 bp, Homo sapiens
  source 1..3212: mol_type = other DNA
gagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaaaaaaaaaa

SEQ ID NO: 4 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 5 - DNA, 27947 bp, synthetic construct
  misc_feature 1..27947: note = Recombinant polynucleotide
  source 1..27947: mol_type = other DNA
gcagagtctaagaaatcgctgtgtttagccctcgccctgggcactgtcctcacgggagctgctgtggctgctgtcttgctttggaagttcagtaagtgcagggagcctcgatcccaccatgtgctcctgcagtccccagtgctctgagccagaccctgctctctgggctattgagacctctggaggccctccgtgaggttcctctcttacataacgaggctgtctctcttcccttctcttgtttagctatgagattgacacatcatggggaaagcatttagaatgtacccagtgctttggggtgcttggtgccacccagcactgtgagcacaggttcttctaccttggggccacacccagttacctgtatctcactgcacagcagtggctgttggggaccaggcccacccctccatgtcccacctcctgcaactgcagcctgagccttcccatcagcctggggtggtgcagacccatgtgccattgtggatccttcaagttacctgtgtggcagagaggacgtgtgagtgccgtccaaacccaaacactgagagggtccttcccattgcccccacggaagtaaggtgccccagtgctaattccacttatacttgctggtggcaaggacacttctcctccttattaaagtgggggattggctgggtgaggtggctcacgcctgttatcccagcactttaagaggccaaggcaggtggaccacctgaggtcaggagtttgagaccacaagcctggccaacatgttgaaactccatctctactaaaaatacaaaaattagtcaggcgtggtggcgtgcacctgtaatcccagctacttaggaggctggggcaggaggatcacttgaacccaggagttggaggttgcagtgagccaagattgtgcccctgcactccagcctgggtgacagaatgagacttcatctcaaaaacaaaacaaaacaaaacacagtggggccaggagttggaggctgcagcgagctacagtaatgccacggtgttcctcactccatgaggctcattgcgtttctcagcctgaagggcacctctcttctgttttctctgcaagtgggcagcaagtgctccaactctgggatagagtgcgactcctcaggtacctgcatcaacccctctaactggtgtgatggcgtgtcacactgccccggcggggaggacgagaatcggtgtggtgagtcagccttgaccttgggaagggactcctctgctcaccttggagacagcagccgggtccaggggcctttgggtgactgggcctggcgtgcgtccagtacgctgacacatgatgtcattgaatccctgctccaggctgagccctggggctcagagaggttgtgtttccggcccaacctcacccagcaggtgggagatgacagggccaccgaggactgtgtcattggaaccacacgtgctctgaactgccacaggaagtcagttaagatgagcaaactgtttataaagttggagatgcaggctaggaacggtggctcatgcctgtaatcccagcactttgggaggccgaggcagatggatcacctgaggtcaggagtttgagaccagcctgaccaatatggtgaaaccttatctccactaaaaatacaaaaattagccaagcgcggtggcgggtgcctgtaattccagctattcaggaggctgaggcaggagaatcacttgaacctgggaggcggaggttgcagtgagctgagatcacgccactgcattccagcctgggagacagagctggctcaaaaaataaattaattaattaaaaacaaaattggagatgcactatgttattttcaaaacaagctgcctttaaagatctatctgttgtcacagggtgggctcatctgtttcattttattttctgtggtttatctatttattcattttaatgaactaggaagcattgctcctatttatggcataccacatgatgtttggatacgtgtatgcctgtggcatggctaagtcaagctagaacatgggccttacctcatatacgtgtcttattaagaacacataaaacctactcttgtagtgattttcaaatatgcaacatatagtttattaactgcagtcactatgatgtacaatagattgctcgaacttattcctcctgtctaactaagattttgtgacctctgaccaacatctccccagtgttgtcaccccccgcccccagcctctgatagctgcctttctactctctgcttctgtgagtttgatgtttatacattccacatgtaagtggcctcatgcagtgtttctgtctctgtgtctggcttgttcacttagcgtaatgtcctccagcttcatctatgttgttggaaatgacaggatttccttctttcttgtggctgaatagtattgccttgtgcatatacaccacattttctttatcccttcattcactgatggactcttaggttgatgtcatgtcttggctgttgtgaaaaatgccgcagtgagcgtgggcgtgcaggtccctcttcaacacacggatttcctttcctttggatataaacccagcagtgagattgctggatcacatggcagttctgtttctcaccttttgaggaaactccatactgttttccataatggctgtagcaacttccactcccacccccacggtgcaaagtctccatttctcttctacaacctcaccaactcctgttattttccatctttctgatagtagccatttgaagaggtatgagatgatacctcattgtggttttcatttgcatttttatttgtatttttcatgaatttttgagggtgatttcaagggtagttagtgactcgaacagggaaacgatcctgagtatgagggttgtgctaatcatccccctcctgccagctgcgtacggaatggggctctgcagatggcagggagctggctcgtttctctttaagagctgccttttacttttcttcctcttcctttaaaacttatttcctggccggacgcagtggctcatgcctgtaatcccagcactttgggaggccgaggtgggcggatcacgaggtcaggaattccagaccagcctggccaacatggtgaaaccccgtctctactaaaaatacaaaaattagccagacgtggtggtgcgggcctatagtcccagctactcgggaggctgaggcaggagaatcacttgaacctgggaggagggggttgcagtgagccgagattgcgccactgcactccagcctgggcgacagagccagactccatctcaaaaaacaaaaaaaagttatttcccaagcacagccatgtattccaggcttgtggatcagcgttggtggtggtgtgtgctctcatatcttagttccagctaagcacactctgacatgtttacactagaaccatttgttttttctagaaatagaaatttcagaattgtagagtcagaggacttaccagaaatctcttaggtagttctcctcccctccctcaagtgcagtcctaacctcctggagttttctgtagaaaccacaagcctcagagctggccgagaattctagccaaagatttttccatgccaaagtaatcccccctctcctaagggccatccttggtggggactggtttcctgttaagccctcgctgtcagtcctggctgtggaatttcctggtgaggagcactggcccgtggagctcggccctcgtgccggccttgagcaggcccaagtgttccgtgttcttgatacctttcctccagcacagtcttgcttcccagaaaaaggtttgcacttgaaaatgatgcatttgctgattaaacatagttcttttgctttatttggtttctaaaataaagtgggagtttttgagattgagtaacgtgaggttaagatagcacgtggaatggctttttcttttctttctattttttttttttttttcctggagacagggtttcactctgttgcccaggctggagtgcagaggcatgaccatggctcactgcaacttcgatgtcctggggttaagcgatcccccagcctcagccccccaagtggctgggactacaggtgctcgccaccacacctggctaatttttgtattttttgtagaaaatgggtttcatcaatgttgtccagactggtctcgaactcctgacctcaagcaattctcctgcctcagcctcccagactgctgggattacaggcgtgaactaccacgcctggcctggaatggcttttgatgttctcctatgtgcacatgtgggtgaataaacaccaacaaagtccttatgttacctgaagagttgctctcttcttaatatttaagtcgtatttatttaaatactttaatagttgtacactattaaagtattattaggtcaaaatcaaggaagtacaaaagggtatgctgtgaaaaatctcttcttccttgctctgcttacttacctaccccgcatccccccatacaccccagacacacacacacacacacacacacacacacacacacgcatcactcccatacatgcccacctgtttaccagccaatcacatttcttggggcaactcatctgagttgcttctctttccagagagtttttgcataaagaagcacaggtatttctgcgttaccatgaccctatttcccagtggttcctagccagttgactctcctgcactggataccatcctggacagcattccttagggaaatgagccccctgttttttcccaccatggcacagttggtcctttgcatggacgcaccattattgcccctgtctcttcttggtggaccttaaggttttctccatccttttgctgtaacacacactgctccaagtgtgtgagcatatcagtaggaaacgcttccaggagtagaactgctaggtcagagggcgtgtggatctgtaacctgacagacctagaccggcttcagtttggttttatccagtttccatattgattattcatataaaaggaaacagacaaacataacgctgtgcatgtattctctcttagaccagaacaggcatagggtgcacttttaatttgtccatttcgtagagtagaaattgtttttgctgaaatgaacaccttaggatgctgaagaatatgacccgtcccatggaaaacattcaaaaatgtgtgtagcgctttcttcccaagggtgtgtgtgcgcatattttaacactaattcactttctacttccgttgctatcctttctgtgagtctttctcagaatctcagaaaagaaactaaattgttcactctagttatcaatgctgtactctatacctggaatttgctaaaagggcagattttaagtattctcaccacagaaaagagaaaagaaaatggtaattatgtgacgtggtggacatgttaactagctttattatggtgagcatttcacagcggatatccagtcatcacgctgtacacattaaacatgtacaattgggtttttttgagacaaggtctccttctgtcacccagtctggagtgcagtggctcagtcatggctcattgcagcctcgacctcctgggctcaatccatccttccccctcagcctcctgaaaagctggggccacaggcatgtaccatcatgccaggctaatgcatatatatttatattttttggtggagatggggttggtctcgaactctgggctcaagtgatcctcccgccttgcccttccaaagtgctgagattacaggcatgaaccacagcaccaggcctacatgtaaaatttttatttgtcaactatactttgacaaagctgagaaaaaaaatcctaatatttaaaaaaaaaaaaaaaaggactagcttgagaccttttccagctctctggcttatcagctgccgtctcttccgggtgcagatagctggaagggaaagaaaatccctaaaattacccacaagccaagaatgaagtgtctccctttgagccacagtggcagttttgtttttaatcatagaagtgtattttgagccgggtgtgctggctcacgcctgtaatccccgcactttgggaggccgaggtggggggcggagggggtggggatcgcctgaggtcaggagttcgagaccagcctgaccaacatggagaaaccccgtctctactaaaaatacaaaattagccggcgtggtggtgcatgcctgtaatcccagctactcatgaggctgagtcaggagaatctcttgaacccaggaggtggaggttgcggtgagctgagatcatgccattgcactccagcctgggaacaagaaaaaaaaagaagaagaagaagaagtgtattcatttcagttacttttaaaaaagtgaacagactttatattttagagcggttttaggtttacagaaaatgaaacagacagggcagcgagctccttgtactcctccccagcacacagttgccctgttatgaacatcccacatcagtgctgtgcgttcattaacaccgatgaacctgatgcatacattatgatgaactgaagtcctggacttcaccctttctcttgtacagttctgtgggatttgacaaatgcataatgctgtacagccacaatgatagtatcgtccagagtagttctcctgccttaaaacctcttttgctgcacctgtttctctctccccactcaccccagctatctgatcttcttagtgcctccgaagttttggtcttttcaggatgttgtagcgttggaatcatggagtatgtagccttcaccacatacaccttccttcactttgttggcttcctttacttagtaatatgcattcaagtttcctccatgccttttcatggcttgatagctcatttctttttagcaccaaataatattccgttgtccagatgtagcacaatgtttatccattcatgtaacctgtgaccgactcacagataggatgtggaatcactcaccacagaggcattagacaataatcagacccaagtcatttcatgggggaacaagcccacaggtaccagactgtccagtgagtcagggccactcgtaggaagtaagaagagaggctagagcatagccaggtcctcactttatactttaagcccatgtgtatttctcccaaaccacacagcattgtttccatgctttcagctttgcatgaataacgtgatacttgaacgcatcatttatcacttgctctctttcccacagcgctgttttcaagcttcttcctgttcatgatgctctgcttaacccttaagctgcatgggattctgttctgtgaatacgcccaccccatgtattatcctgcccagcaaaaagtccccaaaactctggatggtggttacctctagggagggagagaagagattgggaatagggagcgacttcaacggtgtttgtaatgttttgtttctttaaataaaagagctgagatcatttcagcagaatgttgatttagagtctcctggacaatttgttgctcaaagtgctctcttaaagagcactttaaaaaaaaaaaccttttatcttattatttatttatttatttattgagacggagttttgctctgtcacccaggctggagtggagtggtgtgatctcagctcactgcaacctttacctcctgggttcaagcaattcccctgcctcagcctcccaagtaggtgggattacagatgcgtgccaccacacttggctaatttttgcattttagtagagatcggtttctccatgttggccaggctgatctcaaacgcctgacctcaggtgatctgcccgccttggcctcccaaagtgctggtattacaggcgtgagctaccatgcctggcttatcttatatatttttaaaaacagcttattgagatctaatttatgtaccataaaattcaagtatataattcagtgcttttatatataaaacatatatatgaaatagcttattgagatataattttttatataaaacagcttattgatatgtaatgtatgtaccataaaatttaaatatataattcactggcttttatatattcacgaatatgtgcaactatcaccacagtcaattttagcatattttcatcagctcataaagaaaccccaagcccttgaactatcaccccatatccctcctcccagcccgtccctcctactcataagcaaccactaatctacttagtgtctatagatttcctactctaggcattccatgtgagcgggatcatgcaatacgtgggctcacacaatataagtggcattccatgtgagtcggctcatgcagtatgtccggctcctttcactgagcataaggtcttcagcactcatccaggttgcagcctgtgtctgaatttcattccctcttctggctgaatcgtattccattgtgtatcttggacatatcctattctgctcacccagccgttggtgggcgtttggagtgttttcgcctttcagctgttttaagagggttgcagtgaacatttgtacaagttttggacccaatgcctgttttcaattctcttgtgtagagagcactttttagcagaaaaagaatagatttgtggcctccctttgtgtgcggtcagtgccttgagaagagtgaactgtgctgccacctccggagccgtggagagcgcggggcttgggtagcagctaggacgatacaagttgggacaaggccaggtgcaatggctcacgcctgtaattccaacactttgggagaccgaggcagggggatcacctgaggtcaggagttcaagaccagcctggccaacatggtgaaaccccatctctaataaaacagaaaaattaactggacggggtggtggacgcctgtaatcccagctactcgggaggctgaggcaggagaatcacttgaacctgggaggcggaggctgcagtgagtggagatcagaccactgcacttcagcctaggtgacagagcgagactccgtctcaaaaaaaagaaaaaaaaagaaagaaactcatggataatcctccctctcgtgcagttcgcctctacggaccaaacttcatccttcaggtgtactcatctcagaggaagtcctggcaccctgtgtgccaagacgactggaacgagaactacgggcgggcggcctgcagggacatgggctataagtgagtatggggcagcacccgccgagtgacagtaacagacagcagaaacacgagaagaccctctctctgcctccctgtgaaagcaccggcacatgagtgctggggacaattgtcaccttccaaaagctgagccctataaccagcaggtggaatttgtcctgctagggctgtgcccagcacacagaccttggctcactgccaccttgccctgcctcctccttggcctctatagactcctggttgctcgggagtgcccagtgctgtggtcatctggtcagaggggtaggctgagggcgttaggtgcctctttttccaaggtgcctctcagccagggtccattcacctccctgggtagaggttggaccagaacagctggcgaggagggttgggctggggagagcagcagagacaaatcctgtgccagtttcacttcattcgggagccatggaagccttttgagctggggagagaatcaatcaatcagactgatacttaaaaaatgtcattcctgctcgtagctctgagggaaggtgggaaggcttaacagggtgtgtgtcgcctgacagtgattcctaacgggggtggggcggtggttaccatttaccagcactgcctggggagatgcggcagccctcaggcatcgggggagagggtggtaggatgctactgccactttgttttccatgggagggtccccaggtgatttctatgcaactttagggtattcaatatgccagttttcagaatgaattaccactcggtgagaaagttggcatcttagctagtcactgtgacatccctaaacagcaggggtgaattacacagcaaagcccccccatcacagtccaggaacctggtggaattgataactggggccatgttaacatctgtaccttttattagattaaatgtgtgtatgattatacaatcctatgtccttctcatagtttcttgatcctaacctggataagaaacacgaccaatgaaggaattttgtctgacactttagggttattgaatcgaaaaatcgttacaatattctagcacttggttagaacgtgtgattttttttcctaaatgctaaggtttttccctcttattctgaatgtcgtatgagcggtattatgacatagtataggatttgtgtttgcttatgccttaaccattatcacaaataaggttttcttttttaggaataatttttactctagccaaggaatagtggatgacagcggatccaccagctttatgaaactgaacacaagtgccggcaatgtcgatatctataaaaaactgtaccacaggtatgcagcaatttcttcttgaaaaattttggaatgaaatcaactaggagacaccatggggaatcgttgtcctgagtctgatttctctgagctgcaatactcggtctggatgggttttgcattgggaggagattagagtctgaccaggcctggttactctaagcagcccttggtttattcataggaagtggctgaggtttctctgctatttcattttcagcctctaccgtctgcccttgttggtagcggctcacacttgcaacatcgacattcaactctatttagttttctttcctcttcagacatttagaggtgtacctattttgtcagggcgtggttctaggaatccaagataatgtctcagtgtcccagccagggtgaccggctcattccagtttgccagggacttcactggcttgagcaagggaagtcctgctccattccaggcagctgggctggctggtcccgttagccccaaccccgggacagcagtgccagagggtgctctgtgagggatgggcagcattctggcggcctgggaatgagttgtggtgtttccagggggtagaagtgggtacaagccacaggtcacatgatgagtggctgacctggctgggagggcagaagaggggatggacttaggctcttccttttgctttgcacatatttaggatgtttgcagacttgctatgattgttgctgttatgtgttttctgatgtgaaagatacacagtgtcctttgcccatgagctctccttgcctcccaggtccccagggcttatgcctggtgtctaggcatcacctccctgcctgccaggtgccaggtgctgcatttcgggggaggatgaactaatcaccccgcgccacctttcctctgagtgggagcctggggcaggtttgcattcctggaggccgctggtggaggggtctgggggcctgacttccactgcagcctgctgtcctggggaatgtggcagggcaagcccagtggggagggctgtgcacggccaggtgcacccatcaaaacagcagggctgcggtttgtccctgtggagaagctaaacacagctgcctgggcactttgtaaatgctgagtggttctttgtctttctgggttacacacggaatcagggagccaagtccagccgggcagggacggggggaggggaggaggtgctgccgtcccttggcaagagccttgggaactcacaaggaggctggagggcttggaagaaagaagagaaggccattgtctggtaggctctattctatctcggtggtggtggtggggggaggcgcacttcttttcctctttctgtgcagcagttgccctttgatgcctgagttcttggcttgttttctgtcgggcttctgtgaataaccacatgtgccctggcgctgtgaccacacagggctatccctaccgaccttaggattcttaggaaatgtcttctcttaaaggggacatgtcttcacttggccgtgtcagtgccccagagccagagtccacctggaatgcacctgtagtcactgagaacccggggggtgtgccttagtaagaaggtgtcaggaaggacctattattgtagggcctgggctcctgcaaggtggtttgggggtggttggaggaagcagagatttgctctggattggatgctgtcaggaagcaggggtaattctgtgaggctgctttattattttttttctaggaggaggttggaatgaggctaggctaaagctgtgattggtaaagaaacgtccgtcgctcaagttagccaggacaggaggagacatcagatcgtgattttgtggttgtgagcacaaggttcctgttctgtctgttcagacatcatttcggaggaggctccttgtgtcttgccccatctcaggcatggaggggcctagtccgatattgacgctcagtgaaataattcaggttccgcagagcacacggcccagctatcagggcgggccagctctgcatgccaggggccgcgtcttcccttctcagcatagcctgggaaattcactgcaggacaaaatgcatcagttacttcctcttcatccataacctgggatgtttgactcccaaatgagtaactcttacgtttcttctaatcctagggaaactattggttatattgctttcaacactacaaatttaaagcagttataggagcccagaggtttccaaatggcttccttaaaaattagaagatgattttaaattccaagaggaaaaacaaaactagcattattgtatacttaccctcacaaccgtcctaggagctggtacaattttaagagaggttaagtaacttgcccaaggtcacactgtggggatgtgagccgcgtaccttggctcagtgtctggtctttgccactgtccctatatggatttacttaccttattggagttgtaactagcagacccttctatgtctcagaagacaggagagggaacatcggaagaaatgactgatttctaagcatgtgagaggcaggtgactccgcactatcgtgaccagaatttcccctgttctttttgcagtgatgcctgttcttcaaaagcagtggtttctttacgctgtataggtaagttcatctggagtcccccttttgatacttctaactaggaaaagctctctactttcagaacagtactccctgtgtctctgggggcgtgggagggaagaaggtggggtcacgggttggaatgtgcccagcggcgtctcgctctttccaaggagctcctggtttagatttccatggcctgtagacaccttcagccttgggtccaagggacaccccctgagatcaggcacgctcaagaagctgacaaagccctacactttatgccacccatgagctggaggcccggcaggtctctttctccagaaagcaaaggggggtggcgttagtgagccctggcagccacctaacgtggacttggagcatctgcggggctgtggtccagcaccaccgtgtggccaccaggtgctcatcagccagtgggacccgggaggagggacaagaccagagaacaacagtgctcttgcctcttctctcctgaattttggacggtggcttagacttgggtgtccccatctctgtgtttagagtgcttacagtttccaaactgtttgcaaatgtggaagccaccgtccctctcctctgggatggcccagtgctgtcgtggggccgtggtcctgagctcagcttttcatttgaagaggtggaaggagctgacaccgtcccatcccggcagggctggctcaggtcttctttaggtcctgagtgggggtccagcacagccccaagggtgcgtggcacccgccctgccctctgcccatgcactcatctcctggtggagaagacactcacacacaggaagcagggaaggcagcagacctcactcacccctcaccccctcactcaccccctactcaccccctcaacctctcattcaccacccaccccctcgccccctcactcaccccctcactccctcaaccctcactcacctcctcactccctcaaccctcactcacctcctcacctcctcactctccccctcatccctccctcaccccaccccgtcacctcctcactcacctcctcaccccctcactcacccttcaccccctcactcaccacctcacctcctcactcaccccctactcaacccctcattcacccctcaccccctcactcacccctgcaccccctcactcaccccttcatccactcacccacctgctcacctcctcactcaacccctcaccccctcactaatccctcactccctcaccccctcacgccctcactcacaccttcacctcctcactcaccccctcaccccctcaaccccttacttaccccctcactcatcccttcacccctcactcaccccctctctcacccattcaccccctcactcatgccttcaccccctcactcacctcctcactcacaccttcacccctcagtcaccccctcactcaccccttcaccccctcaatcatgccttcactccctcactcaccccttcaccctctgaattactccctcatcccctcactcaccccctcactcaccccttcaccccctcacccaccacctcacccacccctcacccaccccctcacctccttacccctcacccccctcactcacccctcaccccctcactcaccacctcacccacccctcacccaccccctcactcactccctcatcccctcactcaccccctcaccccctcactcaccccctcacccacccctcacccaccccctcaccccctcactcaccccttcaccccctcactcaccccctcactcaccccttcaccccctcactcaccacctcacccacccctcacccaccccctcactcactccctcaccccctcactcaccccctcaccccctcactcaccccctcatctcctcactcaccccctcacctcctcactcacccgctcacctcctcactcaccccctcgccccctcactcacccctcaccccctcaccccctcactcacccctcaccccctcgccccctcactcaccccctcgccccctcactcacccctcaccccctcaccccctcactcatcccctcacctcctcactcaccccctcacctcctcactcaccccctcacctcctcactcaccccctcacctcctcacccaccccctcactcactccctcaccccctcaccccctcactcaccccctcacctcctcactcaccccctcacctcctcacccaccccctcactcactccctcaccccctcaccccctcactcaccccctcacctcctcactcaccccctcacctcctcactcaccccctcacctcctcactcatgccctcaccccctcactcaccctttcacctccttgctcatcccctcacttaccccctcacttcgtcaatcacccccccacctcgtcaatcaccccctcaccttttcactcaccccctcactcacccccttacttcctcacttacctcctcaccccccactcaccccctcaccccccactcaccccctcaccccacactcaccccctcaccccccactcaccccctcacccctctcacctcctcactcaccccctcacctcctcacttatcccctcaccccctcaattaccccctcaccccctcaattactccctcatcctttcaattacccactcaccccctcacctcctcactcctcactcactccctcactcaccccttcaccttctcactcacctcctcgtctcctcaccccctcactcacttccagccctgcccctcccatcttccttttctttgtgtgagaatctggggtccctgagtggtgtcagtccctccaagactcaaggagtccccagggccttgttatccagaacacccccacctgggtcccgggagaccccatgggatcacaggagtgttcagggaagtggtgcttcctgggtctgggtgggctggaggggcatcctcccttccccaagaggagacccccaggagccccctaagtccatccccagcagtggtgcccctgccctgtccttgcagcctgggagacccttgggaggggcgggcgctgggtggctgggcggcttctgctggtctcaccccactggcctcctgtttgtcatcctcagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaagtatgccaggggcggcgcgggccgggtgggggctcagggctggcctacagccaccctgtgaccttgagcaggtctcaacccttgcagccccggcatccttgtgtttaaatggggagagtattgcacctgcttcctagggctgtgagacatcaagtgcgctcatgccaggcagtgcatggctgtatgcactgagtgtcccctgcacgcagggcacagggtgcaggtggaacattctccacgatgtcgccgtgaccagcgttccttccagccactgtcctctgagctctgtcctgcccttgagcaaagcccctgccccctgaggtatcctgtctccgggacgctagtcccaggagagggcacactcagacaggcttcaggctgccctgctggaaggtccctggggttaagcgttcttggccacagcattgctcatgcagagggttaggtaggggtgaggctagccgtgacagtattagcatttatggacgctaccaccccctccccttttccttaaacacatagtgcttttggtcacatgctgctttggaggaggcctcacttggcggatgtatttttctgccttagagagaggctgaactgggtttgactgttggcccagccctctcttgctgcgtgcccttagacgattcactcaacgtctctgatccatggcatgtacaactataagatgggcatgcccttctcctctcgggctgttatgaaggtcaaggaagcaagggctgttacccaagggtgctcccttctctccccctcttcacacccccaggtgctctgggccctctaggaactgggtttctctcaagggctgttacccaagggtgctcccttctctccccctcttcacaccactgggtgctctgggcccactaggagctgggattctcttaagagggaaactcttggataaaggaaatggtttgattgatatcggacaagtctgttcattagtatccatttattaagcacctaccatgtgccaggaaatgctttggcgtacaaaggaaaataagggccagtcctgctagaaatggccttgaaaccccagggagggatgtcggcccattgtgggtgctgcagattccttgaaggtgatgcaagagccagaaagaaggatgatgtggggggctgaggcagggagtcggggttgggggagtgtgggggagaaggggagaccgagcacctcttccactatctccctgtgtggtttttggtgaaccatcctgcctctgggtgtcttgcctccagcttctgacgttggaagttcatccactgagagctctgtgtttatggctctgagatactgagtccttcttctctcccagacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacggtacgtgtggctcaggcttggcaagcaggttggcagaatcttaaagagatgttgattggaaatgacacttgtgctatgccaaatggaagggaggcatttgcgttgagcgagggtagcgtgcagcgggtggccaatgggagaggctcacagaggctaagagcacctgccgcattttgggggaggcagcagccaccacatctgttctgtactgtactgagtggtggtgattcaagccaggcatggaaaaggctagaacagggctttcccactgcagcacccttgacatctgggtggttctctgttgtagggctctcttgtgccttgtaggatgtttaacagcgtccccagcctctacccactggaggccagtagctaccaagctgtgacaaccagtgttgcctgctgacattgccaaacatccgctttgaggcaaagtcacttccagttgagaactactggcctaaaatgtgtaaagatccttgatttttaaagatacattctaaaaccaagttgcttaattcaggacaaacatgctttctcttagcctcttattcggtcccactctggtccatccaagggtctggaatgttctagccccatgtggatacagaagaagcaaaacctcagccctccctacagcatgtctgtattcacattgggaaatggttcacatatagaagagcgaatgcctgagcaatggcgtggtgcctctggggcgaaagctgactccattgactccatcggctttttggctgttgcctcctgtgtgtctttcccgtcttgatcacctggagatatgtaattttggaagcagagctagcaaataattcctcttataagcagagctagcaaataattctacttataagtagcataacgtcttgcctgccagaaggagaggtctggcagggggagaaagtgagaatgtgggacttgttgggatgcagggtcctctgggcagggtggccagggtgccaggcccagcagcctgcatgtgggaaggccaggtggagacataggtgatacccgcctggctcactgtgttttctcttcttgaaacagacctagtgaaaccagtgtgtctgcccaacccaggcatgatgctgcagccagaacagctctgctggatttccgggtggggggccaccgaggagaaaggtgaggctgctcctgggcacacaggactgcagggcccacagatggagcattgggttcggaagtgggaggtccaggttttaatcccagttctactactcaatgactggatgactttggttgattcccccagtccttgtgcctcagtttctccatctgctaagtgggagaaatcctgcccagcctacctaatacactgtgttcttatcgtgatcacacagagcagcatgtggaatggcttttgaagtatctgggccatacgagtttagaggtgcaggatctcctgtgttgcactcattgtgagtttagagctgccctggagatcccaccaaggcctgcgtggctgagtgacagggggcttggtgaggacgggcatcctggacccatggtggccacatctaagcctgtcctctgccctgataaccacagagagaggctctctccacccacttcctttgcaatctgcatttctctctgacagtctttcaaatgaagggagcctggctgcttcatttttatggagggttggaagtgcttagtggcaggcacaaaggttcattttacatattgtttatatccttctcaaaagcgtctaggccatacagacaacaaatcctttcaaacaaggggaaaagtacaaaggttgggtgatttctggggagcgtcagggaaggtagtggggggcatcctggctcctcatcagcagaaacttactacagtagagccacaggctgggcaaaagacctcatggaatccaagatgaagggaatatcgacaaatatttgtgcgcacctgcacctagtacaggctgggtgctactcaggtgctgggaatgcagaagtgaacagagtaagacaaatgtctctgctgtcaggagctttacctctcttctggatgtcggtggtggggacggggcaggtgtggtcagacagatgggagacaaacaactgagcgaggtacttccaaacatctgagggtggggatcacaaggtcccggctattttgaaggggtggtcaggaaaggcttctcggaagaggtggcatttgagctgagactcaaatggcaaaaatgtgtacacatcaaaaaggctagtgcatgtatcttcaggtgtggtcaaggggccaaggaggtgggctggggccagattgcataggtccttgtggattatggtgaagacaccagcttctcatctgcttgaggtggggagatcgtgagccggggagtgccatgatctggcagctgcgtggggagtggggatgaatggatggagacgaggatgatggtgacaagtccattgctgtggttccttgagacaggaagccagctcatagcagagtgcgggcgtggatgtgaagagatgagggtacactagggctagagccaccagacttactgatgggttgcatgtctgtgggagagagagtgagaagtcagggacgatggctttccactctgtggctgaagccccagggtggcgggtggtgccatttttcaagccaggaaatattggttggtgagaatttggggtgggagaaggtgtgacggagggttctggttttgcacactaagcccacggtgcccagaagatgcccgaggggaggcagcaaagcgagagtgggaaatgcagaggtggcaagtgcaggccgtgtcttgagaagctctaatgtgcaggggagccgagaagcaggcggcctagggagggtcacgtgtgctccagaagagtgtgtgcatgccagaggggaaacaggcgcctgtgtgtcctgggtggggttcagtgaggagtgggaaattggttcagcagaaccaagccgttgggtgaataagagggggattccatggcactgatagagccctatagtttcagagctgggaatttctttccctgaagctgaactccagagctgcattcagcacaggcaccgccagttgtaaggagaatccaggtttcccaggagaggggttggtgctgggatgagctgaccggggcagggctggaaaatagggctgtgaccatctgtgtagtgcgtgtggaggtctcagggagggaagtgtgctctccctgcgagagctgcaggcaacactgggagctcaacaagtctccctgtccttagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccaggtaattcaacatttttattctacctttggtccttaccagatcctactgaaccccccatgagagagagggcattcttggggtcagcagagcctcctcagtgacacggagccagctcggggcagtcatgggaagtgacggccacaaacagtgcgaacgcttctggtggcagaaggaagtacagtcaacaaatcacacacaccctctgaaaaaccggtatttggtaaaagtgccagtggaacagaaacaagtatttagactattttaaattatgaacggcaatttatttagtaacttttagcttgaacagattaaaattcaggatgggggctatctctttgggggttacatctctgttaccatcaccccttgatggtggagattcgaagcccacacagtcactcgtaactcacactgcgacccccgccccccaactcctctaggcctggtcagtggtgtgcggcagattgtgacttgattttctgctctctgtaccttgctgtgtcccacagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggtaactatcctgtcctccttctgactgtgttctccgattcctcgagccaaagccagacatctgttaggcgtggttctgctgctggaagctgactggtgaccactggtcagcatgaagcaaactctgcttcctccagccacagccccatccccccagtgtccacccattgcccattgcctctcactggcttcacttgcatatttcccctggtgtttggatgaaaagcgctggggctcagcttgtgtgaaattccttggtgctctgccaaccacacttcgttctggctcagctgactcagctgttccacccaggccacctcacatcaaactttttttttttttttttgagatggagtctcactgtgtcgcccaggctggagtgcagtggcacaatctcgactcactgcaacctttgcctcctgggttcaagtgattctcctgcctcagcctcccaagtagctgggactacaggcatgcgccaccacgcccagctactttttgtatttttagtagagatggggtttctccatgttggccaggctggtctcgaagccctgacctcaggtgattcacccacctcagcctcccacagtgctgggattacaagtgtgaaccacggtgcccggcctcacatgaaacttttgatttatagagagcagagggaagagccggctgtgcccatccttttctggggccatcgagtggctcctgggcagcccccaaggttaggaagggcaggagcagccagggttctctgatgccccagactcaagcacgagggaaggtctcaggggttccatgtgagcctcatggatgtctctgcttagcagagccctggctttgggcattgtccagatagggggtgagaaccagatcttctcatctccaggacctcagacgtatagttttctcagatttctgtgctttctggggctgggctactagtggaagaaagcagtctattctgtcttctcccaaatctcccagatgcccagtctgttgaaggaggagcagaaccagggggcctttcccgctgaggcccgacctgtgtctccttcaaatgacacgcgggactcagggccttcccatgaccatggggcccagggggcgtcacctggcccagggcccagtgctagaaacagatgaccccaggaggaggaggcagggcaggagggaagctggcagggctgggatggtcagccaggctgaggggcggactcgcaccaggatggagctaggaaatgatccaggtgtgtttggcggctgcaggtgggtccgcatggctgtgcagggagggaagggctgcgtggcaggagagcagccgggggaggcccagactctgctgaagagatgcctgttgtgccggcctccacatccgctgcccgctccttccggagctcctgccccgccatgctcagcctgactctgaccaacacgttggagagaagaatgatccctttgtgctattaagcttgcttatttggtttctaagtgcttcatgcgaacctagaggaaaaaattattttccacctttgtttgtcttaagaaaataacacacttttttttttcctatttgaacaggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctagtaactataacggtcctaaggtagcgagctagctccacgtggctttgtcccagacttcctttgtcttcaacaaccttctgcaagaaaaccaagggcctgaattttaacttcctg

SEQ ID NO: 6 - DNA, 25333 bp, synthetic construct
  misc_feature 1..25333: note = Recombinant polynucleotide
  source 1..25333: mol_type = other DNA
gcagagtctaagaaatcgctgtgtttagccctcgccctgggcactgtcctcacgggagctgctgtggctgctgtcttgctttggaagttcagtaagtgcagggagcctcgatcccaccatgtgctcctgcagtccccagtgctctgagccagaccctgctctctgggctattgagacctctggaggccctccgtgaggttcctctcttacataacgaggctgtctctcttcccttctcttgtttagctatgagattgacacatcatggggaaagcatttagaatgtacccagtgctttggggtgcttggtgccacccagcactgtgagcacaggttcttctaccttggggccacacccagttacctgtatctcactgcacagcagtggctgttggggaccaggcccacccctccatgtcccacctcctgcaactgcagcctgagccttcccatcagcctggggtggtgcagacccatgtgccattgtggatccttcaagttacctgtgtggcagagaggacgtgtgagtgccgtccaaacccaaacactgagagggtccttcccattgcccccacggaagtaaggtgccccagtgctaattccacttatacttgctggtggcaaggacacttctcctccttattaaagtgggggattggctgggtgaggtggctcacgcctgttatcccagcactttaagaggccaaggcaggtggaccacctgaggtcaggagtttgagaccacaagcctggccaacatgttgaaactccatctctactaaaaatacaaaaattagtcaggcgtggtggcgtgcacctgtaatcccagctacttaggaggctggggcaggaggatcacttgaacccaggagttggaggttgcagtgagccaagattgtgcccctgcactccagcctgggtgacagaatgagacttcatctcaaaaacaaaacaaaacaaaacacagtggggccaggagttggaggctgcagcgagctacagtaatgccacggtgttcctcactccatgaggctcattgcgtttctcagcctgaagggcacctctcttctgttttctctgcaagtgggcagcaagtgctccaactctgggatagagtgcgactcctcaggtacctgcatcaacccctctaactggtgtgatggcgtgtcacactgccccggcggggaggacgagaatcggtgtggtgagtcagccttgaccttgggaagggactcctctgctcaccttggagacagcagccgggtccaggggcctttgggtgactgggcctggcgtgcgtccagtacgctgacacatgatgtcattgaatccctgctccaggctgagccctggggctcagagaggttgtgtttccggcccaacctcacccagcaggtgggagatgacagggccaccgaggactgtgtcattggaaccacacgtgctctgaactgccacaggaagtcagttaagatgagcaaactgtttataaagttggagatgcaggctaggaacggtggctcatgcctgtaatcccagcactttgggaggccgaggcagatggatcacctgaggtcaggagtttgagaccagcctgaccaatatggtgaaaccttatctccactaaaaatacaaaaattagccaagcgcggtggcgggtgcctgtaattccagctattcaggaggctgaggcaggagaatcacttgaacctgggaggcggaggttgcagtgagctgagatcacgccactgcattccagcctgggagacagagctggctcaaaaaataaattaattaattaaaaacaaaattggagatgcactatgttattttcaaaacaagctgcctttaaagatctatctgttgtcacagggtgggctcatctgtttcattttattttctgtggtttatctatttattcattttaatgaactaggaagcattgctcctatttatggcataccacatgatgtttggatacgtgtatgcctgtggcatggctaagtcaagctagaacatgggccttacctcatatacgtgtcttattaagaacacataaaacctactcttgtagtgattttcaaatatgcaacatatagtttattaactgcagtcactatgatgtacaatagattgctcgaacttattcctcctgtctaactaagattttgtgacctctgaccaacatctccccagtgttgtcaccccccgcccccagcctctgatagctgcctttctactctctgcttctgtgagtttgatgtttatacattccacatgtaagtggcctcatgcagtgtttctgtctctgtgtctggcttgttcacttagcgtaatgtcctccagcttcatctatgttgttggaaatgacaggatttccttctttcttgtggctgaatagtattgccttgtgcatatacaccacattttctttatcccttcattcactgatggactcttaggttgatgtcatgtcttggctgttgtgaaaaatgccgcagtgagcgtgggcgtgcaggtccctcttcaacacacggatttcctttcctttggatataaacccagcagtgagattgctggatcacatggcagttctgtttctcaccttttgaggaaactccatactgttttccataatggctgtagcaacttccactcccacccccacggtgcaaagtctccatttctcttctacaacctcaccaactcctgttattttccatctttctgatagtagccatttgaagaggtatgagatgatacctcattgtggttttcatttgcatttttatttgtatttttcatgaatttttgagggtgatttcaagggtagttagtgactcgaacagggaaacgatcctgagtatgagggttgtgctaatcatccccctcctgccagctgcgtacggaatggggctctgcagatggcagggagctggctcgtttctctttaagagctgccttttacttttcttcctcttcctttaaaacttatttcctggccggacgcagtggctcatgcctgtaatcccagcactttgggaggccgaggtgggcggatcacgaggtcaggaattccagaccagcctggccaacatggtgaaaccccgtctctactaaaaatacaaaaattagccagacgtggtggtgcgggcctatagtcccagctactcgggaggctgaggcaggagaatcacttgaacctgggaggagggggttgcagtgagccgagattgcgccactgcactccagcctgggcgacagagccagactccatctcaaaaaacaaaaaaaagttatttcccaagcacagccatgtattccaggcttgtggatcagcgttggtggtggtgtgtgctctcatatcttagttccagctaagcacactctgacatgtttacactagaaccatttgttttttctagaaatagaaatttcagaattgtagagtcagaggacttaccagaaatctcttaggtagttctcctcccctccctcaagtgcagtcctaacctcctggagttttctgtagaaaccacaagcctcagagctggccgagaattctagccaaagatttttccatgccaaagtaatcccccctctcctaagggccatccttggtggggactggtttcctgttaagccctcgctgtcagtcctggctgtggaatttcctggtgaggagcactggcccgtggagctcggccctcgtgccggccttgagcaggcccaagtgttccgtgttcttgatacctttcctccagcacagtcttgcttcccagaaaaaggtttgcacttgaaaatgatgcatttgctgattaaacatagttcttttgctttatttggtttctaaaataaagtgggagtttttgagattgagtaacgtgaggttaagatagcacgtggaatggctttttcttttctttctattttttttttttttttcctggagacagggtttcactctgttgcccaggctggagtgcagaggcatgaccatggctcactgcaacttcgatgtcctggggttaagcgatcccccagcctcagccccccaagtggctgggactacaggtgctcgccaccacacctggctaatttttgtattttttgtagaaaatgggtttcatcaatgttgtccagactggtctcgaactcctgacctcaagcaattctcctgcctcagcctcccagactgctgggattacaggcgtgaactaccacgcctggcctggaatggcttttgatgttctcctatgtgcacatgtgggtgaataaacaccaacaaagtccttatgttacctgaagagttgctctcttcttaatatttaagtcgtatttatttaaatactttaatagttgtacactattaaagtattattaggtcaaaatcaaggaagtacaaaagggtatgctgtgaaaaatctcttcttccttgctctgcttacttacctaccccgcatccccccatacaccccagacacacacacacacacacacacacacacacacacacgcatcactcccatacatgcccacctgtttaccagccaatcacatttcttggggcaactcatctgagttgcttctctttccagagagtttttgcataaagaagcacaggtatttctgcgttaccatgaccctatttcccagtggttcctagccagttgactctcctgcactggataccatcctggacagcattccttagggaaatgagccccctgttttttcccaccatggcacagttggtcctttgcatggacgcaccattattgcccctgtctcttcttggtggaccttaaggttttctccatccttttgctgtaacacacactgctccaagtgtgtgagcatatcagtaggaaacgcttccaggagtagaactgctaggtcagagggcgtgtggatctgtaacctgacagacctagaccggcttcagtttggttttatccagtttccatattgattattcatataaaaggaaacagacaaacataacgctgtgcatgtattctctcttagaccagaacaggcatagggtgcacttttaatttgtccatttcgtagagtagaaattgtttttgctgaaatgaacaccttaggatgctgaagaatatgacccgtcccatggaaaacattcaaaaatgtgtgtagcgctttcttcccaagggtgtgtgtgcgcatattttaacactaattcactttctacttccgttgctatcctttctgtgagtctttctcagaatctcagaaaagaaactaaattgttcactctagttatcaatgctgtactctatacctggaatttgctaaaagggcagattttaagtattctcaccacagaaaagagaaaagaaaatggtaattatgtgacgtggtggacatgttaactagctttattatggtgagcatttcacagcggatatccagtcatcacgctgtacacattaaacatgtacaattgggtttttttgagacaaggtctccttctgtcacccagtctggagtgcagtggctcagtcatggctcattgcagcctcgacctcctgggctcaatccatccttccccctcagcctcctgaaaagctggggccacaggcatgtaccatcatgccaggctaatgcatatatatttatattttttggtggagatggggttggtctcgaactctgggctcaagtgatcctcccgccttgcccttccaaagtgctgagattacaggcatgaaccacagcaccaggcctacatgtaaaatttttatttgtcaactatactttgacaaagctgagaaaaaaaatcctaatatttaaaaaaaaaaaaaaaaggactagcttgagaccttttccagctctctggcttatcagctgccgtctcttccgggtgcagatagctggaagggaaagaaaatccctaaaattacccacaagccaagaatgaagtgtctccctttgagccacagtggcagttttgtttttaatcatagaagtgtattttgagccgggtgtgctggctcacgcctgtaatccccgcactttgggaggccgaggtggggggcggagggggtggggatcgcctgaggtcaggagttcgagaccagcctgaccaacatggagaaaccccgtctctactaaaaatacaaaattagccggcgtggtggtgcatgcctgtaatcccagctactcatgaggctgagtcaggagaatctcttgaacccaggaggtggaggttgcggtgagctgagatcatgccattgcactccagcctgggaacaagaaaaaaaaagaagaagaagaagaagtgtattcatttcagttacttttaaaaaagtgaacagactttatattttagagcggttttaggtttacagaaaatgaaacagacagggcagcgagctccttgtactcctccccagcacacagttgccctgttatgaacatcccacatcagtgctgtgcgttcattaacaccgatgaacctgatgcatacattatgatgaactgaagtcctggacttcaccctttctcttgtacagttctgtgggatttgacaaatgcataatgctgtacagccacaatgatagtatcgtccagagtagttctcctgccttaaaacctcttttgctgcacctgtttctctctccccactcaccccagctatctgatcttcttagtgcctccgaagttttggtcttttcaggatgttgtagcgttggaatcatggagtatgtagccttcaccacatacaccttccttcactttgttggcttcctttacttagtaatatgcattcaagtttcctccatgccttttcatggcttgatagctcatttctttttagcaccaaataatattccgttgtccagatgtagcacaatgtttatccattcatgtaacctgtgaccgactcacagataggatgtggaatcactcaccacagaggcattagacaataatcagacccaagtcatttcatgggggaacaagcccacaggtaccagactgtccagtgagtcagggccactcgtaggaagtaagaagagaggctagagcatagccaggtcctcactttatactttaagcccatgtgtatttctcccaaaccacacagcattgtttccatgctttcagctttgcatgaataacgtgatacttgaacgcatcatttatcacttgctctctttcccacagcgctgttttcaagcttcttcctgttcatgatgctctgcttaacccttaagctgcatgggattctgttctgtgaatacgcccaccccatgtattatcctgcccagcaaaaagtccccaaaactctggatggtggttacctctagggagggagagaagagattgggaatagggagcgacttcaacggtgtttgtaatgttttgtttctttaaataaaagagctgagatcatttcagcagaatgttgatttagagtctcctggacaatttgttgctcaaagtgctctcttaaagagcactttaaaaaaaaaaaccttttatcttattatttatttatttatttattgagacggagttttgctctgtcacccaggctggagtggagtggtgtgatctcagctcactgcaacctttacctcctgggttcaagcaattcccctgcctcagcctcccaagtaggtgggattacagatgcgtgccaccacacttggctaatttttgcattttagtagagatcggtttctccatgttggccaggctgatctcaaacgcctgacctcaggtgatctgcccgccttggcctcccaaagtgctggtattacaggcgtgagctaccatgcctggcttatcttatatatttttaaaaacagcttattgagatctaatttatgtaccataaaattcaagtatataattcagtgcttttatatataaaacatatatatgaaatagcttattgagatataattttttatataaaacagcttattgatatgtaatgtatgtaccataaaatttaaatatataattcactggcttttatatattcacgaatatgtgcaactatcaccacagtcaattttagcatattttcatcagctcataaagaaaccccaagcccttgaactatcaccccatatccctcctcccagcccgtccctcctactcataagcaaccactaatctacttagtgtctatagatttcctactctaggcattccatgtgagcgggatcatgcaatacgtgggctcacacaatataagtggcattccatgtgagtcggctcatgcagtatgtccggctcctttcactgagcataaggtcttcagcactcatccaggttgcagcctgtgtctgaatttcattccctcttctggctgaatcgtattccattgtgtatcttggacatatcctattctgctcacccagccgttggtgggcgtttggagtgttttcgcctttcagctgttttaagagggttgcagtgaacatttgtacaagttttggacccaatgcctgttttcaattctcttgtgtagagagcactttttagcagaaaaagaatagatttgtggcctccctttgtgtgcggtcagtgccttgagaagagtgaactgtgctgccacctccggagccgtggagagcgcggggcttgggtagcagctaggacgatacaagttgggacaaggccaggtgcaatggctcacgcctgtaattccaacactttgggagaccgaggcagggggatcacctgaggtcaggagttcaagaccagcctggccaacatggtgaaaccccatctctaataaaacagaaaaattaactggacggggtggtggacgcctgtaatcccagctactcgggaggctgaggcaggagaatcacttgaacctgggaggcggaggctgcagtgagtggagatcagaccactgcacttcagcctaggtgacagagcgagactccgtctcaaaaaaaagaaaaaaaaagaaagaaactcatggataatcctccctctcgtgcagttcgcctctacggaccaaacttcatccttcaggtgtactcatctcagaggaagtcctggcaccctgtgtgccaagacgactggaacgagaactacgggcgggcggcctgcagggacatgggctataagtgagtatggggcagcacccgccgagtgacagtaacagacagcagaaacacgagaagaccctctctctgcctccctgtgaaagcaccggcacatgagtgctggggacaattgtcaccttccaaaagctgagccctataaccagcaggtggaatttgtcctgctagggctgtgcccagcacacagaccttggctcactgccaccttgccctgcctcctccttggcctctatagactcctggttgctcgggagtgcccagtgctgtggtcatctggtcagaggggtaggctgagggcgttaggtgcctctttttccaaggtgcctctcagccagggtccattcacctccctgggtagaggttggaccagaacagctggcgaggagggttgggctggggagagcagcagagacaaatcctgtgccagtttcacttcattcgggagccatggaagccttttgagctggggagagaatcaatcaatcagactgatacttaaaaaatgtcattcctgctcgtagctctgagggaaggtgggaaggcttaacagggtgtgtgtcgcctgacagtgattcctaacgggggtggggcggtggttaccatttaccagcactgcctggggagatgcggcagccctcaggcatcgggggagagggtggtaggatgctactgccactttgttttccatgggagggtccccaggtgatttctatgcaactttagggtattcaatatgccagttttcagaatgaattaccactcggtgagaaagttggcatcttagctagtcactgtgacatccctaaacagcaggggtgaattacacagcaaagcccccccatcacagtccaggaacctggtggaattgataactggggccatgttaacatctgtaccttttattagattaaatgtgtgtatgattatacaatcctatgtccttctcatagtttcttgatcctaacctggataagaaacacgaccaatgaaggaattttgtctgacactttagggttattgaatcgaaaaatcgttacaatattctagcacttggttagaacgtgtgattttttttcctaaatgctaaggtttttccctcttattctgaatgtcgtatgagcggtattatgacatagtataggatttgtgtttgcttatgccttaaccattatcacaaataaggttttcttttttaggaataatttttactctagccaaggaatagtggatgacagcggatccaccagctttatgaaactgaacacaagtgccggcaatgtcgatatctataaaaaactgtaccacaggtatgcagcaatttcttcttgaaaaattttggaatgaaatcaactaggagacaccatggggaatcgttgtcctgagtctgatttctctgagctgcaatactcggtctggatgggttttgcattgggaggagattagagtctgaccaggcctggttactctaagcagcccttggtttattcataggaagtggctgaggtttctctgctatttcattttcagcctctaccgtctgcccttgttggtagcggctcacacttgcaacatcgacattcaactctatttagttttctttcctcttcagacatttagaggtgtacctattttgtcagggcgtggttctaggaatccaagataatgtctcagtgtcccagccagggtgaccggctcattccagtttgccagggacttcactggcttgagcaagggaagtcctgctccattccaggcagctgggctggctggtcccgttagccccaaccccgggacagcagtgccagagggtgctctgtgagggatgggcagcattctggcggcctgggaatgagttgtggtgtttccagggggtagaagtgggtacaagccacaggtcacatgatgagtggctgacctggctgggagggcagaagaggggatggacttaggctcttccttttgctttgcacatatttaggatgtttgcagacttgctatgattgttgctgttatgtgttttctgatgtgaaagatacacagtgtcctttgcccatgagctctccttgcctcccaggtccccagggcttatgcctggtgtctaggcatcacctccctgcctgccaggtgccaggtgctgcatttcgggggaggatgaactaatcaccccgcgccacctttcctctgagtgggagcctggggcaggtttgcattcctggaggccgctggtggaggggtctgggggcctgacttccactgcagcctgctgtcctggggaatgtggcagggcaagcccagtggggagggctgtgcacggccaggtgcacccatcaaaacagcagggctgcggtttgtccctgtggagaagctaaacacagctgcctgggcactttgtaaatgctgagtggttctttgtctttctgggttacacacggaatcagggagccaagtccagccgggcagggacggggggaggggaggaggtgctgccgtcccttggcaagagccttgggaactcacaaggaggctggagggcttggaagaaagaagagaaggccattgtctggtaggctctattctatctcggtggtggtggtggggggaggcgcacttcttttcctctttctgtgcagcagttgccctttgatgcctgagttcttggcttgttttctgtcgggcttctgtgaataaccacatgtgccctggcgctgtgaccacacagggctatccctaccgaccttaggattcttaggaaatgtcttctcttaaaggggacatgtcttcacttggccgtgtcagtgccccagagccagagtccacctggaatgcacctgtagtcactgagaacccggggggtgtgccttagtaagaaggtgtcaggaaggacctattattgtagggcctgggctcctgcaaggtggtttgggggtggttggaggaagcagagatttgctctggattggatgctgtcaggaagcaggggtaattctgtgaggctgctttattattttttttctaggaggaggttggaatgaggctaggctaaagctgtgattggtaaagaaacgtccgtcgctcaagttagccaggacaggaggagacatcagatcgtgattttgtggttgtgagcacaaggttcctgttctgtctgttcagacatcatttcggaggaggctccttgtgtcttgccccatctcaggcatggaggggcctagtccgatattgacgctcagtgaaataattcaggttccgcagagcacacggcccagctatcagggcgggccagctctgcatgccaggggccgcgtcttcccttctcagcatagcctgggaaattcactgcaggacaaaatgcatcagttacttcctcttcatccataacctgggatgtttgactcccaaatgagtaactcttacgtttcttctaatcctagggaaactattggttatattgctttcaacactacaaatttaaagcagttataggagcccagaggtttccaaatggcttccttaaaaattagaagatgattttaaattccaagaggaaaaacaaaactagcattattgtatacttaccctcacaaccgtcctaggagctggtacaattttaagagaggttaagtaacttgcccaaggtcacactgtggggatgtgagccgcgtaccttggctcagtgtctggtctttgccactgtccctatatggatttacttaccttattggagttgtaactagcagacccttctatgtctcagaagacaggagagggaacatcggaagaaatgactgatttctaagcatgtgagaggcaggtgactccgcactatcgtgaccagaatttcccctgttctttttgcagtgatgcctgttcttcaaaagcagtggtttctttacgctgtataggtaagttcatctggagtcccccttttgatacttctaactaggaaaagctctctactttcagaacagtactccctgtgtctctgggggcgtgggagggaagaaggtggggtcacgggttggaatgtgcccagcggcgtctcgctctttccaaggagctcctggtttagatttccatggcctgtagacaccttcagccttgggtccaagggacaccccctgagatcaggcacgctcaagaagctgacaaagccctacactttatgccacccatgagctggaggcccggcaggtctctttctccagaaagcaaaggggggtggcgttagtgagccctggcagccacctaacgtggacttggagcatctgcggggctgtggtccagcaccaccgtgtggccaccaggtgctcatcagccagtgggacccgggaggagggacaagaccagagaacaacagtgctcttgcctcttctctcctgaattttggacggtggcttagacttgggtgtccccatctctgtgtttagagtgcttacagtttccaaactgtttgcaaatgtggaagccaccgtccctctcctctgggatggcccagtgctgtcgtggggccgtggtcctgagctcagcttttcatttgaagaggtggaaggagctgacaccgtcccatcccggcagggctggctcaggtcttctttaggtcctgagtgggggtccagcacagccccaagggtgcgtggcacccgccctgccctctgcccatgcactcatctcctggtggagaagacactcacacacaggaagcagggaaggcagcagacctcactcacccctcaccccctcactcaccccctactcaccccctcaacctctcattcaccacccaccccctcgccccctcactcaccccctcactccctcaaccctcactcacctcctcactccctcaaccctcactcacctcctcacctcctcactctccccctcatccctccctcaccccaccccgtcacctcctcactcacctcctcaccccctcactcacccttcaccccctcactcaccacctcacctcctcactcaccccctactcaacccctcattcacccctcaccccctcactcacccctgcaccccctcactcaccccttcatccactcacccacctgctcacctcctcactcaacccctcaccccctcactaatccctcactccctcaccccctcacgccctcactcacaccttcacctcctcactcaccccctcaccccctcaaccccttacttaccccctcactcatcccttcacccctcactcaccccctctctcacccattcaccccctcactcatgccttcaccccctcactcacctcctcactcacaccttcacccctcagtcaccccctcactcaccccttcaccccctcaatcatgccttcactccctcactcaccccttcaccctctgaattactccctcatcccctcactcaccccctcactcaccccttcaccccctcacccaccacctcacccacccctcacccaccccctcacctccttacccctcacccccctcactcacccctcaccccctcactcaccacctcacccacccctcacccaccccctcactcactccctcatcccctcactcaccccctcaccccctcactcaccccctcacccacccctcacccaccccctcaccccctcactcaccccttcaccccctcactcaccccctcactcaccccttcaccccctcactcaccacctcacccacccctcacccaccccctcactcactccctcaccccctcactcaccccctcaccccctcactcaccccctcatctcctcactcaccccctcacctcctcactcacccgctcacctcctcactcaccccctcgccccctcactcacccctcaccccctcaccccctcactcacccctcaccccctcgccccctcactcaccccctcgccccctcactcacccctcaccccctcaccccctcactcatcccctcacctcctcactcaccccctcacctcctcactcaccccctcacctcctcactcaccccctcacctcctcacccaccccctcactcactccctcaccccctcaccccctcactcaccccctcacctcctcactcaccccctcacctcctcacccaccccctcactcactccctcaccccctcaccccctcactcaccccctcacctcctcactcaccccctcacctcctcactcaccccctcacctcctcactcatgccctcaccccctcactcaccctttcacctccttgctcatcccctcacttaccccctcacttcgtcaatcacccccccacctcgtcaatcaccccctcaccttttcactcaccccctcactcacccccttacttcctcacttacctcctcaccccccactcaccccctcaccccccactcaccccctcaccccacactcaccccctcaccccccactcaccccctcacccctctcacctcctcactcaccccctcacctcctcacttatcccctcaccccctcaattaccccctcaccccctcaattactccctcatcctttcaattacccactcaccccctcacctcctcactcctcactcactccctcactcaccccttcaccttctcactcacctcctcgtctcctcaccccctcactcacttccagccctgcccctcccatcttccttttctttgtgtgagaatctggggtccctgagtggtgtcagtccctccaagactcaaggagtccccagggccttgttatccagaacacccccacctgggtcccgggagaccccatgggatcacaggagtgttcagggaagtggtgcttcctgggtctgggtgggctggaggggcatcctcccttccccaagaggagacccccaggagccccctaagtccatccccagcagtggtgcccctgccctgtccttgcagcctgggagacccttgggaggggcgggcgctgggtggctgggcggcttctgctggtctcaccccactggcctcctgtttgtcatcctcagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaagtatgccaggggcggcgcgggccgggtgggggctcagggctggcctacagccaccctgtgaccttgagcaggtctcaacccttgcagccccggcatccttgtgtttaaatggggagagtattgcacctgcttcctagggctgtgagacatcaagtgcgctcatgccaggcagtgcatggctgtatgcactgagtgtcccctgcacgcagggcacagggtgcaggtggaacattctccacgatgtcgccgtgaccagcgttccttccagccactgtcctctgagctctgtcctgcccttgagcaaagcccctgccccctgaggtatcctgtctccgggacgctagtcccaggagagggcacactcagacaggcttcaggctgccctgctggaaggtccctggggttaagcgttcttggccacagcattgctcatgcagagggttaggtaggggtgaggctagccgtgacagtattagcatttatggacgctaccaccccctccccttttccttaaacacatagtgcttttggtcacatgctgctttggaggaggcctcacttggcggatgtatttttctgccttagagagaggctgaactgggtttgactgttggcccagccctctcttgctgcgtgcccttagacgattcactcaacgtctctgatccatggcatgtacaactataagatgggcatgcccttctcctctcgggctgttatgaaggtcaaggaagcaagggctgttacccaagggtgctcccttctctccccctcttcacacccccaggtgctctgggccctctaggaactgggtttctctcaagggctgttacccaagggtgctcccttctctccccctcttcacaccactgggtgctctgggcccactaggagctgggattctcttaagagggaaactcttggataaaggaaatggtttgattgatatcggacaagtctgttcattagtatccatttattaagcacctaccatgtgccaggaaatgctttggcgtacaaaggaaaataagggccagtcctgctagaaatggccttgaaaccccagggagggatgtcggcccattgtgggtgctgcagattccttgaaggtgatgcaagagccagaaagaaggatgatgtggggggctgaggcagggagtcggggttgggggagtgtgggggagaaggggagaccgagcacctcttccactatctccctgtgtggtttttggtgaaccatcctgcctctgggtgtcttgcctccagcttctgacgttggaagttcatccactgagagctctgtgtttatggctctgagatactgagtccttcttctctcccagacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacggtacgtgtggctcaggcttggcaagcaggttggcagaatcttaaagagatgttgattggaaatgacacttgtgctatgccaaatggaagggaggcatttgcgttgagcgagggtagcgtgcagcgggtggccaatgggagaggctcacagaggctaagagcacctgccgcattttgggggaggcagcagccaccacatctgttctgtactgtactgagtggtggtgattcaagccaggcatggaaaaggctagaacagggctttcccactgcagcacccttgacatctgggtggttctctgttgtagggctctcttgtgccttgtaggatgtttaacagcgtccccagcctctacccactggaggccagtagctaccaagctgtgacaaccagtgttgcctgctgacattgccaaacatccgctttgaggcaaagtcacttccagttgagaactactggcctaaaatgtgtaaagatccttgatttttaaagatacattctaaaaccaagttgcttaattcaggacaaacatgctttctcttagcctcttattcggtcccactctggtccatccaagggtctggaatgttctagccccatgtggatacagaagaagcaaaacctcagccctccctacagcatgtctgtattcacattgggaaatggttcacatatagaagagcgaatgcctgagcaatggcgtggtgcctctggggcgaaagctgactccattgactccatcggctttttggctgttgcctcctgtgtgtctttcccgtcttgatcacctggagatatgtaattttggaagcagagctagcaaataattcctcttataagcagagctagcaaataattctacttataagtagcataacgtcttgcctgccagaaggagaggtctggcagggggagaaagtgagaatgtgggacttgttgggatgcagggtcctctgggcagggtggccagggtgccaggcccagcagcctgcatgtgggaaggccaggtggagacataggtgatacccgcctggctcactgtgttttctcttcttgaaacagacctagtgaaaccagtgtgtctgcccaacccaggcatgatgctgcagccagaacagctctgctggatttccgggtggggggccaccgaggagaaaggtgaggctgctcctgggcacacaggactgcagggcccacagatggagcattgggttcggaagtgggaggtccaggttttaatcccagttctactactcaatgactggatgactttggttgattcccccagtccttgtgcctcagtttctccatctgctaagtgggagaaatcctgcccagcctacctaatacactgtgttcttatcgtgatcacacagagcagcatgtggaatggcttttgaagtatctgggccatacgagtttagaggtgcaggatctcctgtgttgcactcattgtgagtttagagctgccctggagatcccaccaaggcctgcgtggctgagtgacagggggcttggtgaggacgggcatcctggacccatggtggccacatctaagcctgtcctctgccctgataaccacagagagaggctctctccacccacttcctttgcaatctgcatttctctctgacagtctttcaaatgaagggagcctggctgcttcatttttatggagggttggaagtgcttagtggcaggcacaaaggttcattttacatattgtttatatccttctcaaaagcgtctaggccatacagacaacaaatcctttcaaacaaggggaaaagtacaaaggttgggtgatttctggggagcgtcagggaaggtagtggggggcatcctggctcctcatcagcagaaacttactacagtagagccacaggctgggcaaaagacctcatggaatccaagatgaagggaatatcgacaaatatttgtgcgcacctgcacctagtacaggctgggtgctactcaggtgctgggaatgcagaagtgaacagagtaagacaaatgtctctgctgtcaggagctttacctctcttctggatgtcggtggtggggacggggcaggtgtggtcagacagatgggagacaaacaactgagcgaggtacttccaaacatctgagggtggggatcacaaggtcccggctattttgaaggggtggtcaggaaaggcttctcggaagaggtggcatttgagctgagactcaaatggcaaaaatgtgtacacatcaaaaaggctagtgcatgtatcttcaggtgtggtcaaggggccaaggaggtgggctggggccagattgcataggtccttgtggattatggtgaagacaccagcttctcatctgcttgaggtggggagatcgtgagccggggagtgccatgatctggcagctgcgtggggagtggggatgaatggatggagacgaggatgatggtgacaagtccattgctgtggttccttgagacaggaagccagctcatagcagagtgcgggcgtggatgtgaagagatgagggtacactagggctagagccaccagacttactgatgggttgcatgtctgtgggagagagagtgagaagtcagggacgatggctttccactctgtggctgaagccccagggtggcgggtggtgccatttttcaagccaggaaatattggttggtgagaatttggggtgggagaaggtgtgacggagggttctggttttgcacactaagcccacggtgcccagaagatgcccgaggggaggcagcaaagcgagagtgggaaatgcagaggtggcaagtgcaggccgtgtcttgagaagctctaatgtgcaggggagccgagaagcaggcggcctagggagggtcacgtgtgctccagaagagtgtgtgcatgccagaggggaaacaggcgcctgtgtgtcctgggtggggttcagtgaggagtgggaaattggttcagcagaaccaagccgttgggtgaataagagggggattccatggcactgatagagccctatagtttcagagctgggaatttctttccctgaagctgaactccagagctgcattcagcacaggcaccgccagttgtaaggagaatccaggtttcccaggagaggggttggtgctgggatgagctgaccggggcagggctggaaaatagggctgtgaccatctgtgtagtgcgtgtggaggtctcagggagggaagtgtgctctccctgcgagagctgcaggcaacactgggagctcaacaagtctccctgtccttagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccaggtaattcaacatttttattctacctttggtccttaccagatcctactgaaccccccatgagagagagggcattcttggggtcagcagagcctcctcagtgacacggagccagctcggggcagtcatgggaagtgacggccacaaacagtgcgaacgcttctggtggcagaaggaagtacagtcaacaaatcacacacaccctctgaaaaaccggtatttggtaaaagtgccagtggaacagaaacaagtatttagactattttaaattatgaacggcaatttatttagtaacttttagcttgaacagattaaaattcaggatgggggctatctctttgggggttacatctctgttaccatcaccccttgatggtggagattcgaagcccacacagtcactcgtaactcacactgcgacccccgccccccaactcctctaggcctggtcagtggtgtgcggcagattgtgacttgattttctgctctctgtaccttgctgtgtcccacagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggtaactatcctgtcctccttctgactgtgttctccgattcctcgagccaaagccagacatctgttaggcgtggttctgctgctggaagctgactggtgaccactggtcagcatgaagcaaactctgcttcctccagccacagccccatccccccagtgtccacccattgcccattgcctctcactggcttcacttgcatatttcccctggtgtttggatgaaaagcgctggggctcagcttgtgtgaaattccttggtgctctgccaaccacacttcgttctggctcagctgactcagctgttccacccaggccacctcacatcaaactttttttttttttttttgagatggagtctcactgtgtcgcccaggctggagtgcagtggcacaatctcgactcactgcaacctttgcctcctgggttcaagtgattctcctgcctcagcctcccaagtagctgggactacaggcatgcgccaccacgcccagctactttttgtatttttagtagagatggggtttctccatgttggccaggctggtctcgaagccctgacctcaggtgattcacccacctcagcctcccacagtgctgggattacaagtgtgaaccacggtgcccggcctcacatgaaacttttgatttatagagagcagagggaagagccggctgtgcccatccttttctggggccatcgagtggctcctgggcagcccccaaggttaggaagggcaggagcagccagggttctctgatgccccagactcaagcacgagggaaggtctcaggggttccatgtgagcctcatggatgtctctgcttagcagagccctggctttgggcattgtccagatagggggtgagaaccagatcttctcatctccaggacctcagacgtatagttttctcagatttctgtgctttctggggctgggctactagtggaagaaagcagtctattctgtcttctcccaaatctcccagatgcccagtctgttgaaggaggagcagaaccagggggcctttcccgctgaggcccgacctgtgtctccttcaaatgacacgcgggactcagggccttcccatgaccatggggcccagggggcgtcacctggcccagggcccagtgctagaaacagatgaccccaggaggaggaggcagggcaggagggaagctggcagggctgggatggtcagccaggctgaggggcggactcgcaccaggatggagctaggaaatgatccaggtgtgtttggcggctgcaggtgggtccgcatggctgtgcagggagggaagggctgcgtggcaggagagcagccgggggaggcccagactctgctgaagagatgcctgttgtgccggcctccacatccgctgcccgctccttccggagctcctgccccgccatgctcagcctgactctgaccaacacgttggagagaagaatgatccctttgtgctattaagcttgcttatttggtttctaagtgcttcatgcgaacctagaggaaaaaattattttccacctttgtttgtcttaagaaaataacacacttttttttttcctatttgaacaggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttctcgagataacttcgtataatgtatgctatacgaagttatgctagtaactataacggtcctaaggtagcgagctagctccacgtggctttgtcccagacttcctttgtcttcaacaaccttctgcaagaaaaccaagggcctgaattttaacttcctg

SEQ ID NO: 7 - protein, 491 aa, synthetic construct
  REGION 1..491: note = Recombinant protein
MALNSGSPPGIGPCYENHGYQSEHICPPRPPVAPNGYNLYPAQYYPSPVPQYAPRITTQASTSVIHTHPKSSGALCTSKSKKSLCLALALGTVLTGAAVAAVLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 8 - DNA, 2267 bp, Mus musculus
  source 1..2267: mol_type = other DNA
ccggttgtgttataggacttgaccagccccaatagtcctcaagtcactcctagatacagtggcaggtggtagctggcttgcggaaggaagaggaagaagagaatgtgggccatcaaggagcaaggccagccttgcacttgggccccctctgctcagtgctgaccagggctttctgagccgcttcctaatgaggctcatttgaagacccccccccacccccctcctgctgtcttgggtggcagagctagctccaggctgtaagaaaattaggaggattaccaaagcagtatggagtcagacagtggccaacccctcaacaaccgtgatattgttccctttcgcaaaccccgaaggccccaggagaccttcaaaaaggtggggatccccatcattgcagtgctgctgagcctgatagccctcgtgattgtggcccttctcatcaaggtgattctggataaatactacttcatctgcggcagtcccctgaccttcattcagaggggccagttgtgtgacggccaccttgactgcgcctcaggggaggatgaggaacactgtgtcaaggacttccctgaaaagcccggagtggcagtccggctctccaaggacagatccaccctgcaggtgctggatgcagccacagggacctgggcctcagtctgtttcgacaacttcacagaagcactggccaagacagcctgcagacagatgggctatgacagccagcccgctttcagagcagtggagatccgtccagatcagaacctccctgttgctcaagtcacaggaaacagccaggaacttcaggtgcagaatggaagcagatcctgcctctcaggctccctggtttccttgcgctgccttgactgtggaaagagcctgaagactcctcgtgtggtgggtggggtggaggcccctgtggattcttggccgtggcaggtcagcatccagtacaacaagcagcatgtctgtggtgggagcatcctggatccccactggatcctcacagcagcccactgcttcaggaagtatcttgatgtgtcaagctggaaggtcagggcaggctcaaacatactgggtaactctccatccttgcctgtggccaagatcttcatcgctgaacccaatcctctgtaccccaaagagaaggacattgcccttgttaagctgcagatgccactcacattctcaggctcagtcaggcccatctgcctgcccttctctgatgaggtgcttgtcccagccacaccagtctgggtcattggatggggctttacagaagaaaacggaggaaagatgtctgacatgctactgcaggcatcagtccaggtcattgacagcacacggtgcaatgcagaggatgcctacgaaggggaagtgaccgctgagatgctgtgtgcaggtaccccacagggtggcaaggacacctgccagggtgacagtggtgggcctttgatgtaccattctgacaagtggcaggtagtaggcatcgtgagctggggccatggatgcggcggcccaagtactcctggagtgtataccaaggtcactgcctatctcaactggatctacaatgttcggaagtctgagatgtaacgctgccgtcccccacatccagaagctgcttcccttcagacctacctacggcatgacccctcaaagtcagatatgggacaagagcctccttgaacaaactctggtatccctgcagcaagcaaggatacattgcagaggtgcccggagtggagtcagatgggctagctcagccacccctgcatctcccaaaccctgggagacatgtggcccatgggagtaaatccaggacattgactcaactctcagaagtgttattcagtcaaggaggctctcccttccactgaaggaaggaaagtcagctctctcctgaaaggccagatcactggctgagtagatgagacaagggtatgaaaggcctttgccatcttctttgcccagtcctgaaagcactgacgtaagagaccagtcagttctaatgtaaggtgtatattttagtgtcagggtattgcaattgtcacctctgtggtcaatatcattaaacaggtatgagaattcgctggcatagacttcctggtctgcttaataagaatccaactaaggatgtcacatgacagtttcccagaaaatgtgaacaagtgtccatctgacacacggcaccaatgacaaaccaaagaagttattctgcctgagtctcagttgctgaactaataaattagctgcggtttcttgca

SEQ ID NO: 9 - protein, 435 aa, Mus musculus
MESDSGQPLNNRDIVPFRKPRRPQETFKKVGIPIIAVLLSLIALVIVALLIKVILDKYYFICGSPLTFIQRGQLCDGHLDCASGEDEEHCVKDFPEKPGVAVRLSKDRSTLQVLDAATGTWASVCFDNFTEALAKTACRQMGYDSQPAFRAVEIRPDQNLPVAQVTGNSQELQVQNGSRSCLSGSLVSLRCLDCGKSLKTPRVVGGVEAPVDSWPWQVSIQYNKQHVCGGSILDPHWILTAAHCFRKYLDVSSWKVRAGSNILGNSPSLPVAKIFIAEPNPLYPKEKDIALVKLQMPLTFSGSVRPICLPFSDEVLVPATPVWVIGWGFTEENGGKMSDMLLQASVQVIDSTRCNAEDAYEGEVTAEMLCAGTPQGGKDTCQGDSGGPLMYHSDKWQVVGIVSWGHGCGGPSTPGVYTKVTAYLNWIYNVRKSEM

SEQ ID NO: 10 - DNA, 3543 bp, Homo sapiens
  source 1..3543: mol_type = other DNA
atcattccagtttggcaacttcacttgtagggctgttttaatcaagctgcccaaagtcccccaatcactcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatggatcctgacagtgatcaacctctgaacagcctcgatgtcaaacccctgcgcaaaccccgtatccccatggagaccttcagaaaggtggggatccccatcatcatagcactactgagcctggcgagtatcatcattgtggttgtcctcatcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagcaaacccactttcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtggggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaaaaaaaaaaaaaaaa

SEQ ID NO: 11 - protein, 437 aa, Homo sapiens
MLQDPDSDQPLNSLDVKPLRKPRIPMETFRKVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGGEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 12 - DNA, 20078 bp, synthetic construct
  misc_feature 1..20078: note = Recombinant polynucleotide
  source 1..20078: mol_type = other DNA
ccacccgcacacactacagtcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagccaagtctgtgtgctaccaagtagcaaaactgagcctggaactcacacatgcgtgtctgagagcccagcactatcgccaggaaaacccagcgtctccctgctcaagcctgaccctcagccctctctgcctctccctgcacttgccttccagtcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcaggtgagtgcagggtctgaggcacaagagaagtgggcccagcaggaggtctgctcaggcccccacggcccactgcatagtatctgccccctacttgtcacttttcatccttgttgtataaggttctttgtttgtttgtttgttgttgttttgaggcagagtgctctgtggcccaagatggagtgcagtgtcttggtctcggctcactgcaacctctgcctcccagtttcaagtgattcttctgcctcagcctcatgagtagctgggattacaggtgccagccaccacgcctggctaatttttatatttttagtagagacggggttttgccacattggtcaggctgatcttgaactcctgacctcaggtgatctgcccgcctcagcctcccaaagtgctgggattacaggcgtgagccaccgtgcccagctgtgtaagtttcttgagagcaggaccctgtcttgtctacctttaaatcctagtacttaacacacagcaaacagtaactatttgatgaccaaatgtgagccagaaaggacaggaaattgtaactgaggctgccccatgcgtgctgcgcctggtggatttcaggcagagggctagactgggtgaccttggggcattcctcctttctatgaaatttgttatttcaaggagactagaaaagagacttctcagccacttcgccagctattggtccttctattcattagtgtttgctgagacatgctatgtgacaggactgagccaggtcctttcaatggataggagatgttttgagcataaaatccacgttctctcttgggctgggctcttctaccttcttccccctggtgcttgggctctgaagaaaaaaagataggtaggagatgagtgatggggcttctgagggcagggctgagtgactttctgtgtatttgctctttctttatcagaagtcaaatgcccacaggcacctgtcatcctactgccagtaggacttctcactcaaccttcccctctgaccttacttggagaaggacttaggtccctctctcagacatttccccaggctgggcaagttgtgtggaccatggatgggtatgtggtccatacaatttaaacaagctgtatatggtcgctgggtagagtgaccacataattgatcatcaaaactgatacctgtaagagcaaaagggggcactattaaccattgggtcagggcaacaggtcaaaatggagacctaccctgggacttctggtcacactagctactgtcaaaatggggcccaaatagacaaagccaaatggaagaaattcccttgacattgaaagtgttggggctctgtggcacccccagttctaggttgggggagcttgggctggtctcatgatgagttctgagggggatgggccagttgggccccccgttccatctaactcaggttcctttcctcccagtccgcctctccaaggaccgatccacactgcaggtgctggactcggccacagggaactggttctctgcctgtttcgacaacttcacagaagctctcgctgagacagcctgtaggcagatgggctacagcaggtaaccaacctgggcctctctcctttttccctccttcctccttcctcctcttcctcctttccttcctcccttcttctctctttcctaaaaattacgggcattggagccaggcagaatggcttttgaatcccagcatttcacttataagcaacatgaagttaaatttcctaagcctcaggttcctcaggagttaattgggggaactaatgccaacctcataggatagttttgcaatgccagtgagagaatgtgtgctgccctccaacacacacacacacacttctagcgtctatgcagtcctctcctttcctttactcctcaaccttcactcctttgtgctggctttgcaagaaactgttcctgcccagtaatacaaaagctaagttaacttattcaaagtttcgttagttaagatttagcttaagtgagcctagtttcagtggggccccatcttcagcaatcccagctctctctgcaaatttcaaaagcagttccaaatctggagtggatgaaaaggtgtaagatgatagtaagagtaatttgcattctatatatttatattcacttgattttggcagaaaaccaaaaagatagttattatatcttatatatagatatatattatatctatttcataaataggctcaaacaaagtaagtaacttgctagggtactagctgggaggtagagggctagaatttgagcccaagacccctaattcttgcgcattaggagttcccacattgtttctgtttctagactgagtaattctttattctcatgtaggacatcatctctaagggaaggggctaatgagatggttgatcactcagagagtttagctggagaggatggaaaagaacccatacattcagttgcagattgagatagcctatctctggcaggcctcagatttcttcaggattctaacagactggacccagagactaggccaaacaaacaaacaaacaaaaactctactaggcagacatcaccaaccaatcacagaactctctcccatggatccctaatacagcctcaaagtccttttcagtaaatgctccaggcagccattacaaatcaatcagaattatttgcctttctcttctctgctcaacgggcttctgctgctctctactttccatagggggcaacttccattaccctctagaaagcacaccccaccaccttcatttcaaggagagtgaggaactcatgcccagcacctgctattctcccctcttcctgcagccacggagcccagcctcgctgcagccagccctgcctccccactgtagtccagtcaactgctgcatcagccgttcctggcacagcaggctgagccttgattatgaaacctgggtgtctccaggggttcttaagatgataggctcctggaatttctgtccttttggagctcagtaaggcaccaaaccacctgagtcttgtgcttcacaaaatcaaagttcatcagaatcattcattgggatggaattggtgaacagaagttaactttcctgggaatgtccatttccaccatattccgtccttctaggtctcagacttctctactttctttcctctctctagatcggaggcccttcttgtcctagaaccataggcatttcaagatgtgggagaccctagggatcatctagtccacgcatcttttttttttttttttgacagagtctcactctgtcacccaggctggagtgcaatggcaccatctctgcttactgcaacctccacctcccaggttcaagtgattctttcgcctcagcctcccaagtagctgggattacaggcacgcaccatcatgcccagctaatttttatatttttgtagagaccgagtttcaccatgttggccaggctggtcttgaactcctgacctcaggtgatccacccacctcggcctcccaaagtgctgggattacaggcgtgagccactgcacccagccccgtgcatctttttatagagggggaaactgaggcttggagagacccagaaaaagaatatgacctgcccaaggccacacatcaaactagtgccagagccagggacagaacctagatcatgaggactcttaaaatgcactctagtcctcccaggtctgagacttgggtccttccaggaagtgccagcattcctgcctgagaatgtgccaatccaccagtattgccaatgactcagccctccatggagagcttctactaacattactagcatagttagggatggaaggaaaagatttagaagaggcagattcagtaaaggaacaatcagagagatggaattaatcaaggaaggcttcctggaggaggaaaaacttcaacccaaggtttgaaagtagcaagcatggattagcagggagaaagagggagagtggtccagttgagagaaacgtttgtctggattcatatgaagacagatctagtcctgttctattaaatatctctaagggggccaaaaacatacccccgctatcaaagtcagaccagatgctttgtttggagaacgaaatatccacattccaactccctcccaggtgagaagggagctaacctgagcccctatgcctctttgtttccctgctgtgaaccagaagacattgctgggatatttgaaatagggacagagctgggaatatggaaaggagacccctaacatttctccagggctctgggttctggatttggattccccacccaagaaagcaagttacatcagcaatgcactgagggttgagtcctgggatgccaagggtcggttctttattgtatagcaaagcaggccccatcttcactgactaagaccatctccactccctggccactccccaccaagcattctctgccactctttctcctgaaagtgggggccaactctaccatcttgttctaaccccctgccccagctcacaactctctctccctcttgatgtgagcagcaaacccactttcagagctgtggagattggcccagaccaggatctggatgttgttgaaatcacagaaaacagccaggagcttcgcatgcggaactcaagtgggtaagtgaggggacaccttctggcctacagaaggcccccacatggacgctgctcttcaggttgcaaccagctcacctggaaccccaagcagccaggggaatgtaagcagacatcaggaagaactcctagccagatggatcattcaatgccaagagctatagactcacattttggagaggttttctgtgttgacttgtttttaatacaatggacagctggacaaagtgtgttgtcctactcagagccagagggatggataatgtgacctttccatcaatctggatagtaaatagtttttgctactgctgtaggttttctaataaattgcccaataggcaagattccaaagtcactttgtccttccctaccacttacccagccagagctccccaccttcttgatgctccagggaagaggctccatggcccttgtgggtggcctgttcctgagcctcgccaccctgtgttagagcagagcatccagatgaaatctgtcacactgtggcaaagtggctcagagaggaggctggcttcctagcattcagggacgttgctgagggccgcttattcaccgaaaataaatcttgaaaaggacagggctggtagcagaatgatcctttacctaaaattctatcaaaatcccattcttccatttggaaagcccacagtgtcacagactctgttccgggctctgtcctcttccctcttgggtcccaggagcccaggctgggctttgaagcaggcagggcccagcacacagtaggtactcagcagtgggggtgttgaatccaatcaaacggaagtgtcaatgcaggaaatgcaatggatgtcaatgcagtctccaaatgttccccactgtgcagcttccacattcccgaggtattgggaggggacttgaattaacagcttcgggaggcctgagtccctgcctcccagctgaggaagaagcttaaatcacagggcgctgtgtctgtcttccaggccctgtctctcaggctccctggtctccctgcactgtcttggtgagtacccccaatctctgagggtttggggcctgggccagcaatgagcagggaggaagaccttcatcttcactcctaaatttctgggactccaagtttcattctgccttggtctacagcccttgggcttgtcggtcaatgccccctcgagttgttggtggccttgggcaggtcacattctttttctgggtctttccaagccccagtttcccccttctaccatctgtgcatggctccatgacctaagtggagacctgggagagagtgttaggaagaccgaaaagggcaggacggggcctccactgcctcccatccctggtccgggcccacatagccttctttgtcacaatcagctcaggtatccaagatcagattacccacattcattatttgagcaactattcattgaacagttagaatatgtctcactctgtcagttgctggctagaagtagaaagtaccagatgagtgaaataattggccactatccttggtagctgatgactaagtaagagagagatgcaagacaacatgtggaaaatgccaaactgagtagcagtcacagttgacatgctgcagagagagctggccgggggtcagaagacctgggcaccagtcctgttcatttccagtgtggcctcgagtcattcacctgacctccctgaagttcattttcccaagaagttgtttagtccaactgcccatcaaggatctttagggacccttctagctctaacagaggagatcagaaaagaaaacaagcaatgtggctcagctcatcctacaagcttcatagagaactgagactggcctggaagcatagccagaaattagaacgcctaagggaagaaggtcacaacgctgcctctgcaatttaggagtgtatatgctttcctgcaggatgttgagagtttcattcattatcgtatgccccctaccccggccccacaatacctagtgcgtgggatctgacacgtggtggctggtcaatgaatgaatgaatgaatggtcacaccatctgaggttctgcactgagtagccctgaaggcttgaagcagcataagtgacaggtcctcccttgaggggcctctgttttaccaataagccaagacctaagctcaacaacactgaaagggtggccaatacccaggacagcctgtgggaattccagagaaagggagattcccagggactgggggcccaggctaaacactgaaaaatgcatctgtaggctcaaggaggaaaagcccatgtctgtctgtcttgcccaccactctctcccagcacccagcactgccccaggacagagagcacttgacacaagttggttagattaatgaatgatttagagttcagtggtccccaacctttttggcacaagagactggttgcatggaagacaatttttccgcaaaccaagagggggatagagagcattagattctctcttttttttttttttgagaccaagtctggctcttgtcactcagcctggagtaaagtgttgcgatctcggctcactgcaacctccgcctcctggattcaagcgattctcctgcctcagccccctaaatagctgggattacaggcacccgtcaccagcccagctgggactataggcatgtgccaccatgcccggctaatttttgtatttttagtagagacggcgtttcaccatgttggccaggctagtctcgaactcctgacctcaggtgatctgcccgcctgagcctcccaaagtgctgggattacaggcatgagctgcctcacccagcctaaagtctcataaggaacgtacagcatagatccctcacatgtgcagttcacaataaggttgtgctcctacaagaatctaacgccacctctgatctgacaggaggtgaagctcaggtggtcatgctcgcttgtccctgccactcacttcctaatgtacagccaggttcctaacaggccacgaaccagtgggaagggcatctttttggatcaaaaacagaattactttttagagaactacaagcagatcaatttggctagacagagactttatatgaaacagcaggaggctgctaggaggagtggaaactctactttgccctcaagggagatcccgaagggctttgcaggagcgggcaaggtggcatgaagaaagcagtgtttgaaatcaggtggtatttgaaaagcccagcccttccccttagaatggcccttctaccatctgtgcatggctccacaaccgtggtggtggctgccagaagaattggaaaggcagagcatgggtggagaggggggacctgagggctttacaggagttccgggggtggtgagggtgtgaaagccaggtcagtcagtaggaagacaggatgtcagattgagagactcccctggccggggaaacagacttggagaagggggagttttggatgagacagtccacttccgagtcacaaaatagcttgtgggtgtctgtttactgttactcagtgggagtggctggggacacgccacctgggcagggctttcgtaattctgcatcacttgtgaaggtcacagattcccagcacaacggacacacccatgttcatagtctgaactcctaaacacatcttaaaccaaaataaaaaaaaaagaaagaaagaaagaaaaaggagagggaggtttgaggaaagcctatggtctgggacactcaatacctcccatgaatatctcatattgggctggtcctctctccactctggccccagccataagggccctgcttagagcagattttgggtgctgagtggaggcagcctcatccccaacagcctgacttcctgcctcctccctgcctctgcctgtgtccagcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggtaagaccccagctgtaaggaggtctctggggaccaaggccagtcagggaccagagagcttggggtcctgtctcctggcaccgtccttctcttcactctcccactagagacgttttccaggttgtggtggccccaatgagacaatggccatgatgccctttgttaggcttttgggtgtctgagcagagggtgctggtcaccaagcatggcctcttcctggtgggacaccagcagatacccagagtcctcaccccacccccatatcgttcaagctacaaaagctcttcccacctgcctcaacttccaagaactcactctctttttgcttgtttccaggaagttgttccagggtctagagtcatagccacgtcctcattatgtctggaaactttaaaaaaattaaagagcataggttcctttcagtccacagagaagcctggccttacctcagggaagggctactcccagacccccttcactttttttttttttttttttttttttttttttgagacagagtcttgctctgttgcttaggctggagcgcagcagcatgatcttggctcactgcaacctccgcctcctgagttcaagcaattctcctgcctcagcttcccaagtagctgggactataggcatgggccaccatgcccggctaatttttgtatttttggtagagacagggtttcaccatgttggccaggctgatctctaactcctgacctcaagtgatctgcccacctcagcctcccaaactgctgggattacaggcatgagccagggcatccggcttttatttattcattcattcaatatctaatgagcacctaccaggtaccaaacaccagatgatgcgcccaagttcattagaccccaccgctgtcttcaaggcactcatgatctaggccagcgttttttaaccactttttttttttttttttttgagattctggtgagagctataaattctttcctggaaaaacatctctgcacactaagctgtgcctggcattgggaaaaagaaagcacgtaatgtaactgacagcatgagtaacacagtgagaaaggttggaggagagagcgccaggacctcagaactcaggcattagaggagccccttccccagccctccttgaggtttcgttgggcaggtttcactgaggaaaaagggtcaaatccctttttcgaatttgacttcttgtaagtgccagaagactgccccttctccaccatccctgcctcaccatcatctttcctcccaaggcagtgacatccagcaccccgatccctagggccctggggacccagcctttggcaaagtctcctcaggcttggatcaggcctgaacccagctgtctctacccccaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggtgagaagcagggcccaaggccactcaagcctcttacatcagttttcacgcccactctgctattagctcactgaccgcccttggcacataatgtctcctctcaagtcctcagcttgcccatttgtctctaatacgtcagcctaacatcactgatgccatgaggcctcctcaagctgtcagctaacacctccactccattccctgccagagattcttccaaggcctgtcttccctatgtggagcccctcgagtgagaactggagtttcatccaatcttggagttttaggagaccttttaaaaagattatcgagctaattccccaccactgaccaacacgcaagagcctgctcagtatccctgccaaggagtcattgtgcccctgtttgctctcctccaggggcagggaacccattacctgtgaggcagcccacagagtctttgaacagctctgttggatgccttgtgcttatactgaaatgtatttagatcaggattcccaactgtggggtccacaagacactggccccttggagaagagaggattccattgtcaaataagtttggggaacattttcatactacagctcccttcttggaacacattagtttattaaaggtaggagaagtttttaaaataatctgttttattgcgtttaacctacattttttaaatttatttgaccacagaatccttttttcatgctacttctattagcatcccatagaacaagtgttctagagaccctggtgtgacccctttcagagagcttaactgccaggctctcctgagccctggtgtgtgtttcaagatttgtgcctgggaattgttttaatcaggtatggcaaggtgacagatacagacacagctatctttgaaagaagagtttattatttataattcctgagagaaagggacataccccaccccccaacacagggacacccggggaagcagctgggtccaccaggaggcaggagtgaggggaaggcatggcccagagccacctgtggcttccatgggcaggtctggccaaggtagggtaggcaagattgagcatgctcaggattggatagtgtggacaattctctaggctatagatgtcagcctctggttgtctagtatctgtccctggggtgatttagggcagggaaaatattggcttggtgtctgagagtcagataaaggaagtggttggggatatgggctttgggttggctggtttgcctattaaaggcgtgcccaaagccaagttgtttactatctgcaggaattagctaacccagtctctcccagaccagcaagatccccataatcataaagcatcataatttacagaaaattaacacttatgatgaataaaagatctccttcttcctctgtgctcctggcaggcacagtcaggcccatctgtctgcccttctttgatgaggagctcactccagccaccccactctggatcattggatggggctttacgaagcagaatggaggtaagtcctgggtgcaggaccacagggcaggagatgcccttgtatgagggagcagcttccagaagtaatgggaaggaggaccacccttcagagaaacccatcctggaggaccaagcaccaaggcgccaggcagaaagcaaagtggtttggcaatccagggctgggggatagaaggcaaggatgggaatgtgagtgtttttaccctcccagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccaggtggggcctccaagaatcatggggagttctaagaatagggtttaggtcctagagagatgagaaaacccagaggctgcatgccctacaggaagccttgcatatcatgggcactcaatgtgtgatgatgggaggaagagagggagggaaggaaaggatagtcagataaaagtgtaccaatagatgagtgggtggatggatggatgcagacaagcagagagatttcaaatgtctctttcacattcgaagatgatgttactggcctggcatggtggctcacgcttgtaatcccagcactttgggaggctgaggcgggcaggtgatttgaggtcaggaattcaagaccagcctggccaacatggtgaaatcccatctctactaaaaagaatacaaaaattagctgggcgtggtggcacgtgcctgtaatcccagctacttgggaggctgaggcaggagaattgcttgaacccaggaggcagaggttgcagtaagctgagattgcgccactgcactccagcctgggtgacccagcaagactccatctgaaaacaacaacaacaacaaagatgacattactcatccaccccacccacccttctcactagctacagaatgattagccccttgaggtcaggaatcccaggtctattttctctgtgactctccccaagctgctgaactacactaggaaagaattaccgcctgcagaatgctggaagcacatctgtgtgtgccctcaccccggcctcattggccatcaggactgcttagcaatccctgtagaccttcttcctcccccatacttccagaggatcttctgaactattttctttttttattttttcttttatgttttttaacagagacagggtcactatgttgcccagtctggtctcaaactcctgggttcaagggattctcccacctcagctttccaaaatgctgggattacaggcatgagccatcgtgcttggcctgaaccattttcattaaaacccctaccctactctcacctccatttccagtcattaaattccttcatttaagaggcatctcttagtcatcgcatgtgtgccatgaacatggtagtctttggagacccctcagggagctcacagtggttgggggaaaggggggcattaaacagacatttaagctatagttttgggttcagagggaggaagccccaggggctaaaacagctgataaggactcccagataagtgcacttttcactatctggcattttcttgttttgttatttgcttgttcactgtctctcaccccatttgatcctaagctttctgagggcagggatctttgttttttttcatcagttggatcccaattgcttagaacactacctggcacaaaataggcactctataagtgattacacaaattttggaacgactaggttaaacaatgataaccaggcttttttttttttttttgagactgagtctcactctgttgcccaggctagagtgaagtggtttgatctcggctcactgcagcctccgcctctgggttcgaatgattctccacctcagcctcctgagtagctgggattacaggtgcctgccactatgcccagctaatttttgtatttgtagtagagacgggtttcaccatgttggccaggctggtcttgaactcctgacctcaagtgattcacccgcctcagcctcccaaggtgctgggattacaggtgtgagccaccgctcctggccaacaaccaggcttttttaagacatcactcagagcctttaatttgctaatgtgagttgtgaatctctgagagaaggctaacggcatgcttgcaacttacttgtccacagacaagcctttctgccccagaagagaagaccattctagggtgctaatgagcaaagagggtgagggtggaatatcggagagcagcagggagtgcaggggaacagataggccagttcagggagcagagaaggagaagcccccccacctcacctgccctccccagcagtctctgttctggtctctcacagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggtaaggtacctttgccctacccactgtgccttccctccagtcctctacctggggggtgccaatccatcctcaggtttgatttaaatggttctgacaactctttacatcccaaataactttccctccaagcaagggacagcctgagattgcactattaaggctgaaattccttaggtcagagatttctgataaatgcaaataccttagggaatagaacacaccaagcctttctttctcttttctgacagaatgagactatcagatcctttctagagagaagattctgataaggaagagagtggaaaggctcatgagacctcctggccctctgcagggtagggagagaagcaaagtgtttcagaaaaggaagactcacgttacacatgtcaccactttgtccagtttcagataatctgactttctcttcatcggtctctcttattctaggctgagctgtaacgctgccgtcccccacatccagaagctgcttcccttcagacctacctacggcatgacccctcaaagtcagatatgggacaagagcctccttgaacaaactc

SEQ ID NO: 13 - DNA, 15159 bp, synthetic construct
  misc_feature 1..15159: note = Recombinant polynucleotide
  source 1..15159: mol_type = other DNA
ccacccgcacacactacagtcgagataacttcgtataatgtatgctatacgaagttatgctagtaactataacggtcctaaggtagcgagctagccaagtctgtgtgctaccaagtagcaaaactgagcctggaactcacacatgcgtgtctgagagcccagcactatcgccaggaaaacccagcgtctccctgctcaagcctgaccctcagccctctctgcctctccctgcacttgccttccagtcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcaggtgagtgcagggtctgaggcacaagagaagtgggcccagcaggaggtctgctcaggcccccacggcccactgcatagtatctgccccctacttgtcacttttcatccttgttgtataaggttctttgtttgtttgtttgttgttgttttgaggcagagtgctctgtggcccaagatggagtgcagtgtcttggtctcggctcactgcaacctctgcctcccagtttcaagtgattcttctgcctcagcctcatgagtagctgggattacaggtgccagccaccacgcctggctaatttttatatttttagtagagacggggttttgccacattggtcaggctgatcttgaactcctgacctcaggtgatctgcccgcctcagcctcccaaagtgctgggattacaggcgtgagccaccgtgcccagctgtgtaagtttcttgagagcaggaccctgtcttgtctacctttaaatcctagtacttaacacacagcaaacagtaactatttgatgaccaaatgtgagccagaaaggacaggaaattgtaactgaggctgccccatgcgtgctgcgcctggtggatttcaggcagagggctagactgggtgaccttggggcattcctcctttctatgaaatttgttatttcaaggagactagaaaagagacttctcagccacttcgccagctattggtccttctattcattagtgtttgctgagacatgctatgtgacaggactgagccaggtcctttcaatggataggagatgttttgagcataaaatccacgttctctcttgggctgggctcttctaccttcttccccctggtgcttgggctctgaagaaaaaaagataggtaggagatgagtgatggggcttctgagggcagggctgagtgactttctgtgtatttgctctttctttatcagaagtcaaatgcccacaggcacctgtcatcctactgccagtaggacttctcactcaaccttcccctctgaccttacttggagaaggacttaggtccctctctcagacatttccccaggctgggcaagttgtgtggaccatggatgggtatgtggtccatacaatttaaacaagctgtatatggtcgctgggtagagtgaccacataattgatcatcaaaactgatacctgtaagagcaaaagggggcactattaaccattgggtcagggcaacaggtcaaaatggagacctaccctgggacttctggtcacactagctactgtcaaaatggggcccaaatagacaaagccaaatggaagaaattcccttgacattgaaagtgttggggctctgtggcacccccagttctaggttgggggagcttgggctggtctcatgatgagttctgagggggatgggccagttgggccccccgttccatctaactcaggttcctttcctcccagtccgcctctccaaggaccgatccacactgcaggtgctggactcggccacagggaactggttctctgcctgtttcgacaacttcacagaagctctcgctgagacagcctgtaggcagatgggctacagcaggtaaccaacctgggcctctctcctttttccctccttcctccttcctcctcttcctcctttccttcctcccttcttctctctttcctaaaaattacgggcattggagccaggcagaatggcttttgaatcccagcatttcacttataagcaacatgaagttaaatttcctaagcctcaggttcctcaggagttaattgggggaactaatgccaacctcataggatagttttgcaatgccagtgagagaatgtgtgctgccctccaacacacacacacacacttctagcgtctatgcagtcctctcctttcctttactcctcaaccttcactcctttgtgctggctttgcaagaaactgttcctgcccagtaatacaaaagctaagttaacttattcaaagtttcgttagttaagatttagcttaagtgagcctagtttcagtggggccccatcttcagcaatcccagctctctctgcaaatttcaaaagcagttccaaatctggagtggatgaaaaggtgtaagatgatagtaagagtaatttgcattctatatatttatattcacttgattttggcagaaaaccaaaaagatagttattatatcttatatatagatatatattatatctatttcataaataggctcaaacaaagtaagtaacttgctagggtactagctgggaggtagagggctagaatttgagcccaagacccctaattcttgcgcattaggagttcccacattgtttctgtttctagactgagtaattctttattctcatgtaggacatcatctctaagggaaggggctaatgagatggttgatcactcagagagtttagctggagaggatggaaaagaacccatacattcagttgcagattgagatagcctatctctggcaggcctcagatttcttcaggattctaacagactggacccagagactaggccaaacaaacaaacaaacaaaaactctactaggcagacatcaccaaccaatcacagaactctctcccatggatccctaatacagcctcaaagtccttttcagtaaatgctccaggcagccattacaaatcaatcagaattatttgcctttctcttctctgctcaacgggcttctgctgctctctactttccatagggggcaacttccattaccctctagaaagcacaccccaccaccttcatttcaaggagagtgaggaactcatgcccagcacctgctattctcccctcttcctgcagccacggagcccagcctcgctgcagccagccctgcctccccactgtagtccagtcaactgctgcatcagccgttcctggcacagcaggctgagccttgattatgaaacctgggtgtctccaggggttcttaagatgataggctcctggaatttctgtccttttggagctcagtaaggcaccaaaccacctgagtcttgtgcttcacaaaatcaaagttcatcagaatcattcattgggatggaattggtgaacagaagttaactttcctgggaatgtccatttccaccatattccgtccttctaggtctcagacttctctactttctttcctctctctagatcggaggcccttcttgtcctagaaccataggcatttcaagatgtgggagaccctagggatcatctagtccacgcatcttttttttttttttttgacagagtctcactctgtcacccaggctggagtgcaatggcaccatctctgcttactgcaacctccacctcccaggttcaagtgattctttcgcctcagcctcccaagtagctgggattacaggcacgcaccatcatgcccagctaatttttatatttttgtagagaccgagtttcaccatgttggccaggctggtcttgaactcctgacctcaggtgatccacccacctcggcctcccaaagtgctgggattacaggcgtgagccactgcacccagccccgtgcatctttttatagagggggaaactgaggcttggagagacccagaaaaagaatatgacctgcccaaggccacacatcaaactagtgccagagccagggacagaacctagatcatgaggactcttaaaatgcactctagtcctcccaggtctgagacttgggtccttccaggaagtgccagcattcctgcctgagaatgtgccaatccaccagtattgccaatgactcagccctccatggagagcttctactaacattactagcatagttagggatggaaggaaaagatttagaagaggcagattcagtaaaggaacaatcagagagatggaattaatcaaggaaggcttcctggaggaggaaaaacttcaacccaaggtttgaaagtagcaagcatggattagcagggagaaagagggagagtggtccagttgagagaaacgtttgtctggattcatatgaagacagatctagtcctgttctattaaatatctctaagggggccaaaaacatacccccgctatcaaagtcagaccagatgctttgtttggagaacgaaatatccacattccaactccctcccaggtgagaagggagctaacctgagcccctatgcctctttgtttccctgctgtgaaccagaagacattgctgggatatttgaaatagggacagagctgggaatatggaaaggagacccctaacatttctccagggctctgggttctggatttggattccccacccaagaaagcaagttacatcagcaatgcactgagggttgagtcctgggatgccaagggtcggttctttattgtatagcaaagcaggccccatcttcactgactaagaccatctccactccctggccactccccaccaagcattctctgccactctttctcctgaaagtgggggccaactctaccatcttgttctaaccccctgccccagctcacaactctctctccctcttgatgtgagcagcaaacccactttcagagctgtggagattggcccagaccaggatctggatgttgttgaaatcacagaaaacagccaggagcttcgcatgcggaactcaagtgggtaagtgaggggacaccttctggcctacagaaggcccccacatggacgctgctcttcaggttgcaaccagctcacctggaaccccaagcagccaggggaatgtaagcagacatcaggaagaactcctagccagatggatcattcaatgccaagagctatagactcacattttggagaggttttctgtgttgacttgtttttaatacaatggacagctggacaaagtgtgttgtcctactcagagccagagggatggataatgtgacctttccatcaatctggatagtaaatagtttttgctactgctgtaggttttctaataaattgcccaataggcaagattccaaagtcactttgtccttccctaccacttacccagccagagctccccaccttcttgatgctccagggaagaggctccatggcccttgtgggtggcctgttcctgagcctcgccaccctgtgttagagcagagcatccagatgaaatctgtcacactgtggcaaagtggctcagagaggaggctggcttcctagcattcagggacgttgctgagggccgcttattcaccgaaaataaatcttgaaaaggacagggctggtagcagaatgatcctttacctaaaattctatcaaaatcccattcttccatttggaaagcccacagtgtcacagactctgttccgggctctgtcctcttccctcttgggtcccaggagcccaggctgggctttgaagcaggcagggcccagcacacagtaggtactcagcagtgggggtgttgaatccaatcaaacggaagtgtcaatgcaggaaatgcaatggatgtcaatgcagtctccaaatgttccccactgtgcagcttccacattcccgaggtattgggaggggacttgaattaacagcttcgggaggcctgagtccctgcctcccagctgaggaagaagcttaaatcacagggcgctgtgtctgtcttccaggccctgtctctcaggctccctggtctccctgcactgtcttggtgagtacccccaatctctgagggtttggggcctgggccagcaatgagcagggaggaagaccttcatcttcactcctaaatttctgggactccaagtttcattctgccttggtctacagcccttgggcttgtcggtcaatgccccctcgagttgttggtggccttgggcaggtcacattctttttctgggtctttccaagccccagtttcccccttctaccatctgtgcatggctccatgacctaagtggagacctgggagagagtgttaggaagaccgaaaagggcaggacggggcctccactgcctcccatccctggtccgggcccacatagccttctttgtcacaatcagctcaggtatccaagatcagattacccacattcattatttgagcaactattcattgaacagttagaatatgtctcactctgtcagttgctggctagaagtagaaagtaccagatgagtgaaataattggccactatccttggtagctgatgactaagtaagagagagatgcaagacaacatgtggaaaatgccaaactgagtagcagtcacagttgacatgctgcagagagagctggccgggggtcagaagacctgggcaccagtcctgttcatttccagtgtggcctcgagtcattcacctgacctccctgaagttcattttcccaagaagttgtttagtccaactgcccatcaaggatctttagggacccttctagctctaacagaggagatcagaaaagaaaacaagcaatgtggctcagctcatcctacaagcttcatagagaactgagactggcctggaagcatagccagaaattagaacgcctaagggaagaaggtcacaacgctgcctctgcaatttaggagtgtatatgctttcctgcaggatgttgagagtttcattcattatcgtatgccccctaccccggccccacaatacctagtgcgtgggatctgacacgtggtggctggtcaatgaatgaatgaatgaatggtcacaccatctgaggttctgcactgagtagccctgaaggcttgaagcagcataagtgacaggtcctcccttgaggggcctctgttttaccaataagccaagacctaagctcaacaacactgaaagggtggccaatacccaggacagcctgtgggaattccagagaaagggagattcccagggactgggggcccaggctaaacactgaaaaatgcatctgtaggctcaaggaggaaaagcccatgtctgtctgtcttgcccaccactctctcccagcacccagcactgccccaggacagagagcacttgacacaagttggttagattaatgaatgatttagagttcagtggtccccaacctttttggcacaagagactggttgcatggaagacaatttttccgcaaaccaagagggggatagagagcattagattctctcttttttttttttttgagaccaagtctggctcttgtcactcagcctggagtaaagtgttgcgatctcggctcactgcaacctccgcctcctggattcaagcgattctcctgcctcagccccctaaatagctgggattacaggcacccgtcaccagcccagctgggactataggcatgtgccaccatgcccggctaatttttgtatttttagtagagacggcgtttcaccatgttggccaggctagtctcgaactcctgacctcaggtgatctgcccgcctgagcctcccaaagtgctgggattacaggcatgagctgcctcacccagcctaaagtctcataaggaacgtacagcatagatccctcacatgtgcagttcacaataaggttgtgctcctacaagaatctaacgccacctctgatctgacaggaggtgaagctcaggtggtcatgctcgcttgtccctgccactcacttcctaatgtacagccaggttcctaacaggccacgaaccagtgggaagggcatctttttggatcaaaaacagaattactttttagagaactacaagcagatcaatttggctagacagagactttatatgaaacagcaggaggctgctaggaggagtggaaactctactttgccctcaagggagatcccgaagggctttgcaggagcgggcaaggtggcatgaagaaagcagtgtttgaaatcaggtggtatttgaaaagcccagcccttccccttagaatggcccttctaccatctgtgcatggctccacaaccgtggtggtggctgccagaagaattggaaaggcagagcatgggtggagaggggggacctgagggctttacaggagttccgggggtggtgagggtgtgaaagccaggtcagtcagtaggaagacaggatgtcagattgagagactcccctggccggggaaacagacttggagaagggggagttttggatgagacagtccacttccgagtcacaaaatagcttgtgggtgtctgtttactgttactcagtgggagtggctggggacacgccacctgggcagggctttcgtaattctgcatcacttgtgaaggtcacagattcccagcacaacggacacacccatgttcatagtctgaactcctaaacacatcttaaaccaaaataaaaaaaaaagaaagaaagaaagaaaaaggagagggaggtttgaggaaagcctatggtctgggacactcaatacctcccatgaatatctcatattgggctggtcctctctccactctggccccagccataagggccctgcttagagcagattttgggtgctgagtggaggcagcctcatccccaacagcctgacttcctgcctcctccctgcctctgcctgtgtccagcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggtaagaccccagctgtaaggaggtctctggggaccaaggccagtcagggaccagagagcttggggtcctgtctcctggcaccgtccttctcttcactctcccactagagacgttttccaggttgtggtggccccaatgagacaatggccatgatgccctttgttaggcttttgggtgtctgagcagagggtgctggtcaccaagcatggcctcttcctggtgggacaccagcagatacccagagtcctcaccccacccccatatcgttcaagctacaaaagctcttcccacctgcctcaacttccaagaactcactctctttttgcttgtttccaggaagttgttccagggtctagagtcatagccacgtcctcattatgtctggaaactttaaaaaaattaaagagcataggttcctttcagtccacagagaagcctggccttacctcagggaagggctactcccagacccccttcactttttttttttttttttttttttttttttttgagacagagtcttgctctgttgcttaggctggagcgcagcagcatgatcttggctcactgcaacctccgcctcctgagttcaagcaattctcctgcctcagcttcccaagtagctgggactataggcatgggccaccatgcccggctaatttttgtatttttggtagagacagggtttcaccatgttggccaggctgatctctaactcctgacctcaagtgatctgcccacctcagcctcccaaactgctgggattacaggcatgagccagggcatccggcttttatttattcattcattcaatatctaatgagcacctaccaggtaccaaacaccagatgatgcgcccaagttcattagaccccaccgctgtcttcaaggcactcatgatctaggccagcgttttttaaccactttttttttttttttttttgagattctggtgagagctataaattctttcctggaaaaacatctctgcacactaagctgtgcctggcattgggaaaaagaaagcacgtaatgtaactgacagcatgagtaacacagtgagaaaggttggaggagagagcgccaggacctcagaactcaggcattagaggagccccttccccagccctccttgaggtttcgttgggcaggtttcactgaggaaaaagggtcaaatccctttttcgaatttgacttcttgtaagtgccagaagactgccccttctccaccatccctgcctcaccatcatctttcctcccaaggcagtgacatccagcaccccgatccctagggccctggggacccagcctttggcaaagtctcctcaggcttggatcaggcctgaacccagctgtctctacccccaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggtgagaagcagggcccaaggccactcaagcctcttacatcagttttcacgcccactctgctattagctcactgaccgcccttggcacataatgtctcctctcaagtcctcagcttgcccatttgtctctaatacgtcagcctaacatcactgatgccatgaggcctcctcaagctgtcagctaacacctccactccattccctgccagagattcttccaaggcctgtcttccctatgtggagcccctcgagtgagaactggagtttcatccaatcttggagttttaggagaccttttaaaaagattatcgagctaattccccaccactgaccaacacgcaagagcctgctcagtatccctgccaaggagtcattgtgcccctgtttgctctcctccaggggcagggaacccattacctgtgaggcagcccacagagtctttgaacagctctgttggatgccttgtgcttatactgaaatgtatttagatcaggattcccaactgtggggtccacaagacactggccccttggagaagagaggattccattgtcaaataagtttggggaacattttcatactacagctcccttcttggaacacattagtttattaaaggtaggagaagtttttaaaataatctgttttattgcgtttaacctacattttttaaatttatttgaccacagaatccttttttcatgctacttctattagcatcccatagaacaagtgttctagagaccctggtgtgacccctttcagagagcttaactgccaggctctcctgagccctggtgtgtgtttcaagatttgtgcctgggaattgttttaatcaggtatggcaaggtgacagatacagacacagctatctttgaaagaagagtttattatttataattcctgagagaaagggacataccccaccccccaacacagggacacccggggaagcagctgggtccaccaggaggcaggagtgaggggaaggcatggcccagagccacctgtggcttccatgggcaggtctggccaaggtagggtaggcaagattgagcatgctcaggattggatagtgtggacaattctctaggctatagatgtcagcctctggttgtctagtatctgtccctggggtgatttagggcagggaaaatattggcttggtgtctgagagtcagataaaggaagtggttggggatatgggctttgggttggctggtttgcctattaaaggcgtgcccaaagccaagttgtttactatctgcaggaattagctaacccagtctctcccagaccagcaagatccccataatcataaagcatcataatttacagaaaattaacacttatgatgaataaaagatctccttcttcctctgtgctcctggcaggcacagtcaggcccatctgtctgcccttctttgatgaggagctcactccagccaccccactctggatcattggatggggctttacgaagcagaatggaggtaagtcctgggtgcaggaccacagggcaggagatgcccttgtatgagggagcagcttccagaagtaatgggaaggaggaccacccttcagagaaacccatcctggaggaccaagcaccaaggcgccaggcagaaagcaaagtggtttggcaatccagggctgggggatagaaggcaaggatgggaatgtgagtgtttttaccctcccagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccaggtggggcctccaagaatcatggggagttctaagaatagggtttaggtcctagagagatgagaaaacccagaggctgcatgccctacaggaagccttgcatatcatgggcactcaatgtgtgatgatgggaggaagagagggagggaaggaaaggatagtcagataaaagtgtaccaatagatgagtgggtggatggatggatgcagacaagcagagagatttcaaatgtctctttcacattcgaagatgatgttactggcctggcatggtggctcacgcttgtaatcccagcactttgggaggctgaggcgggcaggtgatttgaggtcaggaattcaagaccagcctggccaacatggtgaaatcccatctctactaaaaagaatacaaaaattagctgggcgtggtggcacgtgcctgtaatcccagctacttgggaggctgaggcaggagaattgcttgaacccaggaggcagaggttgcagtaagctgagattgcgccactgcactccagcctgggtgacccagcaagactccatctgaaaacaacaacaacaacaaagatgacattactcatccaccccacccacccttctcactagctacagaatgattagccccttgaggtcaggaatcccaggtctattttctctgtgactctccccaagctgctgaactacactaggaaagaattaccgcctgcagaatgctggaagcacatctgtgtgtgccctcaccccggcctcattggccatcaggactgcttagcaatccctgtagaccttcttcctcccccatacttccagaggatcttctgaactattttctttttttattttttcttttatgttttttaacagagacagggtcactatgttgcccagtctggtctcaaactcctgggttcaagggattctcccacctcagctttccaaaatgctgggattacaggcatgagccatcgtgcttggcctgaaccattttcattaaaacccctaccctactctcacctccatttccagtcattaaattccttcatttaagaggcatctcttagtcatcgcatgtgtgccatgaacatggtagtctttggagacccctcagggagctcacagtggttgggggaaaggggggcattaaacagacatttaagctatagttttgggttcagagggaggaagccccaggggctaaaacagctgataaggactcccagataagtgcacttttcactatctggcattttcttgttttgttatttgcttgttcactgtctctcaccccatttgatcctaagctttctgagggcagggatctttgttttttttcatcagttggatcccaattgcttagaacactacctggcacaaaataggcactctataagtgattacacaaattttggaacgactaggttaaacaatgataaccaggcttttttttttttttttgagactgagtctcactctgttgcccaggctagagtgaagtggtttgatctcggctcactgcagcctccgcctctgggttcgaatgattctccacctcagcctcctgagtagctgggattacaggtgcctgccactatgcccagctaatttttgtatttgtagtagagacgggtttcaccatgttggccaggctggtcttgaactcctgacctcaagtgattcacccgcctcagcctcccaaggtgctgggattacaggtgtgagccaccgctcctggccaacaaccaggcttttttaagacatcactcagagcctttaatttgctaatgtgagttgtgaatctctgagagaaggctaacggcatgcttgcaacttacttgtccacagacaagcctttctgccccagaagagaagaccattctagggtgctaatgagcaaagagggtgagggtggaatatcggagagcagcagggagtgcaggggaacagataggccagttcagggagcagagaaggagaagcccccccacctcacctgccctccccagcagtctctgttctggtctctcacagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggtaaggtacctttgccctacccactgtgccttccctccagtcctctacctggggggtgccaatccatcctcaggtttgatttaaatggttctgacaactctttacatcccaaataactttccctccaagcaagggacagcctgagattgcactattaaggctgaaattccttaggtcagagatttctgataaatgcaaataccttagggaatagaacacaccaagcctttctttctcttttctgacagaatgagactatcagatcctttctagagagaagattctgataaggaagagagtggaaaggctcatgagacctcctggccctctgcagggtagggagagaagcaaagtgtttcagaaaaggaagactcacgttacacatgtcaccactttgtccagtttcagataatctgactttctcttcatcggtctctcttattctaggctgagctgtaacgctgccgtcccccacatccagaagctgcttcccttcagacctacctacggcatgacccctcaaagtcagatatgggacaagagcctccttgaacaaactc

SEQ ID NO: 14 - protein, 435 aa, synthetic construct
  REGION 1..435: note = Recombinant protein
MESDSGQPLNNRDIVPFRKPRRPQETFKKVGIPIIAVLLSLIALVIVALLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 15 - DNA, 2046 bp, Mus musculus
  source 1..2046: mol_type = other DNA
cagaaacaaggacctcttcattattcaagagtaaaatgtataggccaagaccaatgctatcaccgtcaagattcttcactccctttgcagtagctttcgttgtcataataacggtagggctcctggccatgatggcaggtctacttattcactttttagcttttgacaagaaagcttacttttatcatagcagctttcaaatcctaaacgttgaatacactgaggctttaaactcaccagctacacacgaatacagaaccttgagtgaaagaattgaggctatgattactgatgaatttcgaggatcaagtctaaaaagtgagtttatcaggacacatgttgtcaaactaagaaaagaagggactggtgtggttgcggatgttgtcatgaaatttcgatctagtaaacgtaacaacagaaaggtaatgaaaaccagaattcaatctgtgctacgaagactcagcagctctggaaacttggaaatagccccttcgaatgagataacatcactcactgaccaggatacagaaaatgttttgactcaagaatgtggagcacgtccagaccttataacactgtcagaagagagaatcattggaggcatgcaagctgagcccggtgactggccctggcaagtcagtctacagctcaataatgtccaccactgtggaggtgccctgatcagtaacatgtgggtcctgacagcagctcattgcttcaaaagctatcctaatcctcaatattggacagccacctttggggtttctacaatgagccctaggctgagagtgagagtaagggctattttagcccacgacgggtacagctccgtaactcgtgacaatgacatcgcagttgtacaacttgacagatctgtcgccttttccagaaatatccatagggtatgtctcccagcagcaacccaaaatatcatccctggttctgtcgcatatgttacaggatggggatctctcacatatggaggcaacgcagtcacaaatctacggcaaggagaggtcagaataataagttcagaggaatgcaatacgccagctggttacagtggaagtgtcttgccaggaatgctgtgtgctggaatgcgttcaggggccgtggatgcatgccagggtgattcaggtggcccgctagtacaagaagactcaaggcggctttggtttgttgtgggcattgtgagctggggatatcagtgtggcctcccaaataagccaggcgtgtatactcgagtgacagcctaccgcaactggatcagacagcagacgggaatctagtgcaaccgaggaaaaaacgtgccatgaggtctctgtatccaagtgtgactgactcggatgccatggcttcacatttcaactgcaaaggagactggaaatgccccttctgaacgtcccattacataaatatggtttaactgtttagtatttctttgtcggtacagatttttactttcttgaggaaaaaaaaaacatgaacatggctaagtaagaattatgttaggctagtaacaggaagacatttattacatgggtggtcaggtgtagtagtgagaagtcaggtaagttaagtcaataatttacagaaaataatgtcaggtagtcctaacgttaaatatgtgaggccacagaacaaatagtgttagaactgaagccatcccaagtatttaacatttgttttcaagtgaaactaagaaacagacttacatatagttttaatggtgaattttcattttaaatattttatctacatagaaaagacatatctccttcatgaagaagctgaggtgatgaatcaacacagcctcttcagctatgtttgcaaccacaagatttgtgggaaagaaatccctactaccaacttcctactgttggcattattttttagagtaacacgacgcacaatagcaaaatttaagtaacaaattaaaagttaatgatgaagaagaagtaaagagtttgtttgcaaagacaaaaattaaacagattaatatcaataaatctggagacagaagggtctcagattcatattctctct

SEQ ID NO: 16 - protein, 417 aa, Mus musculus
MYRPRPMLSPSRFFTPFAVAFVVIITVGLLAMMAGLLIHFLAFDKKAYFYHSSFQILNVEYTEALNSPATHEYRTLSERIEAMITDEFRGSSLKSEFIRTHVVKLRKEGTGVVADVVMKFRSSKRNNRKVMKTRIQSVLRRLSSSGNLEIAPSNEITSLTDQDTENVLTQECGARPDLITLSEERIIGGMQAEPGDWPWQVSLQLNNVHHCGGALISNMWVLTAAHCFKSYPNPQYWTATFGVSTMSPRLRVRVRAILAHDGYSSVTRDNDIAVVQLDRSVAFSRNIHRVCLPAATQNIIPGSVAYVTGWGSLTYGGNAVTNLRQGEVRIISSEECNTPAGYSGSVLPGMLCAGMRSGAVDACQGDSGGPLVQEDSRRLWFVVGIVSWGYQCGLPNKPGVYTRVTAYRNWIRQQTGI

SEQ ID NO: 17 - DNA, 2800 bp, Homo sapiens
  source 1..2800: mol_type = other DNA
atttgagtgggaatctcaaagcagttgagtaggcagaaaaaagaacctcttcattaaggattaaaatgtataggccagcacgtgtaacttcgacttcaagatttctgaatccatatgtagtatgtttcattgtcgtcgcaggggtagtgatcctggcagtcaccatagctctacttgtttactttttagcttttgatcaaaaatcttacttttataggagcagttttcaactcctaaatgttgaatataatagtcagttaaattcaccagctacacaggaatacaggactttgagtggaagaattgaatctctgattactaaaacattcaaagaatcaaatttaagaaatcagttcatcagagctcatgttgccaaactgaggcaagatggtagtggtgtgagagcggatgttgtcatgaaatttcaattcactagaaataacaatggagcatcaatgaaaagcagaattgagtctgttttacgacaaatgctgaataactctggaaacctggaaataaacccttcaactgagataacatcacttactgaccaggctgcagcaaattggcttattaatgaatgtggggccggtccagacctaataacattgtctgagcagagaatccttggaggcactgaggctgaggagggaagctggccgtggcaagtcagtctgcggctcaataatgcccaccactgtggaggcagcctgatcaataacatgtggatcctgacagcagctcactgcttcagaagcaactctaatcctcgtgactggattgccacgtctggtatttccacaacatttcctaaactaagaatgagagtaagaaatattttaattcataacaattataaatctgcaactcatgaaaatgacattgcacttgtgagacttgagaacagtgtcacctttaccaaagatatccatagtgtgtgtctcccagctgctacccagaatattccacctggctctactgcttatgtaacaggatggggcgctcaagaatatgctggccacacagttccagagctaaggcaaggacaggtcagaataataagtaatgatgtatgtaatgcaccacatagttataatggagccatcttgtctggaatgctgtgtgctggagtacctcaaggtggagtggacgcatgtcagggtgactctggtggcccactagtacaagaagactcacggcggctttggtttattgtggggatagtaagctggggagatcagtgtggcctgccggataagccaggagtgtatactcgagtgacagcctaccttgactggattaggcaacaaactgggatctagtgcaacaagtgcatccctgttgcaaagtctgtatgcaggtgtgcctgtcttaaattccaaagctttacatttcaactgaaaaagaaactagaaatgtcctaatttaacatcttgttacataaatatggtttaacaaacactgtttaacctttctttattattaaaggttttctattttctccagagaactatatgaatgttgcatagtactgtggctgtgtaacagaagaaacacactaaactaattacaaagttaacaatttcattacagttgtgctaaatgcccgtagtgagaagaacaggaaccttgagcatgtatagtagaggaacctgcacaggtctgatgggtcagaggggtcttctctgggtttcactgaggatgagaagtaagcaaactgtggaaacatgcaaaggaaaaagtgatagaataatattcaagacaaaaagaacagtatgaggcaagagaaataatatgtatttaaaatttttggttactcaatatcttatacttagtatgagtcctaaaattaaaaatgtgaaactgttgtactatacgtataacctaaccttaattattctgtaagaacatgcttccataggaaatagtggataattttcagctatttaaggcaaaagctaaaatagttcactcctcaactgagacccaaagaattatagatatttttcatgatgacccatgaaaaatatcactcatctacataaaggagagactatatctattttatagagaagctaagaaatatacctacacaaacttgtcaggtgctttacaactacatagtactttttaacaacaaaataataattttaagaatgaaaaatttaatcatcgggaagaacgtcccactacagacttcctatcactggcagttatatttttgagcgtaaaagggtcgtcaaacgctaaatctaagtaacgaattgaaagtttaaagagggggaagagttggtttgcaaaggaaaagtttaaatagcttaatatcaatagaatgatcctgaagacagaaaaaactttgtcactcttcctctctcattttctttctctctctctccccttctcatacacatgcctcccccaccaaagaatataatgtaaattaaatccactaaaatgtaatggcatgaaaatctctgtagtctgaatcactaatattcctgagtttttatgagctcctagtacagctaaagtttgcctatgcatgatcatctatgcgtcagagcttcctccttctacaagctaactccctgcatctgggcatcaggactgctccatacatttgctgaaaacttcttgtatttcctgatgtaaaattgtgcaaacacctacaataaagccatctacttttagggaaagggagttgaaaatgcaaccaactcttggcgaactgtacaaacaaatctttgctatactttatttcaaataaattctttttaaaataaaaaaaaaaaaaaaaaaa

SEQ ID NO: 18 - protein, 418 aa, Homo sapiens
MYRPARVTSTSRFLNPYVVCFIVVAGVVILAVTIALLVYFLAFDQKSYFYRSSFQLLNVEYNSQLNSPATQEYRTLSGRIESLITKTFKESNLRNQFIRAHVAKLRQDGSGVRADVVMKFQFTRNNNGASMKSRIESVLRQMLNNSGNLEINPSTEITSLTDQAAANWLINECGAGPDLITLSEQRILGGTEAEEGSWPWQVSLRLNNAHHCGGSLINNMWILTAAHCFRSNSNPRDWIATSGISTTFPKLRMRVRNILIHNNYKSATHENDIALVRLENSVTFTKDIHSVCLPAATQNIPPGSTAYVTGWGAQEYAGHTVPELRQGQVRIISNDVCNAPHSYNGAILSGMLCAGVPQGGVDACQGDSGGPLVQEDSRRLWFIVGIVSWGDQCGLPDKPGVYTRVTAYLDWIRQQTGI

SEQ ID NO: 19 - DNA, 38992 bp, synthetic construct
  misc_feature 1..38992: note = Recombinant polynucleotide
  source 1..38992: mol_type = other DNA
gagggagggtggtgctttgctaatggtgaattactaactcctcaataaagaatattatttgaaataatttttgaaatttcataattactttgggttctttcttaatgataaataaataatagtatattacaaacatacattaatatttcctgaatgaatacaccacaaatctcccttaaaatatagcaagaataaaaattatactatttctgacaatttttaatttctcaaataataataccactctgatttttaaacatctacaccactctggctttgccaatctttttaaaaattgaaaagataataattttatcataattacactgaagcatagaactttttctttcaaggaaagcaaatttttgaaattctataatataacctcccataatcctgaataaattaaaggttcaacaacttagtaaagtaagactgaccttcccttttatttctttttcagatcaaaaatcttacttttataggagcagttttcaactcctaaatgttgaatataatagtcagttaaattcaccagctacacaggaatacaggactttgagtggaagaattgaatctctggtaagttaatatttgtctttgctctttattccattataaaatgaatatgataataaacctaatgttttgtaatatattttcagttgctaagtgctctacatattttccttccttgaatggtgaaacatgtgtttctctctgcttttatccagttagtttactcatatactggttcttattcacatctttgtcatgagtaaaaagtgttagaaaggccacgagtaaatatgcattttatttgtttatgaattcaaatactaaaagttttttatttgtttaattaagcattgacattgtctttttaaattcttttcattttaccttcttccctcttccttatccaactaaagacgcaaagcaggaggtgttaaaaaacaggtttaccatatcagcagtaacatagtttggacaacattacactttggttcaatgatagacatagaagtttgaacagaaatatgcaaagcaagtttgagctctaacttgaagagagcctctgggtgcctgccaggaaacctcacgagtggacccttaacattcatgtgtcaccacaaactaggggctgccctttagttttgaccagtctcagtgtcactcacttacccttaccttttcaaaaaaaagtcctaagaatataaagtaattcaatggttctacaattttagcatgtaactgagtcacctggcagggttgctttggtgagctcaagataaaattttatcagcatttctacattttctggaatattccttaatccaggcttttaatcccttggtgcttttctgaaccactgcaatgagcttctaactgttctcactgtgtgcaggctcttttccttctaatctaatttacacacttctgaacacaaatctctcacagcctgtttccttcatgttacctccagctcaagactttttgcctacaaaataaaattcaaacttgttagctaagcaccttctcatgtctatgctttggctcatatttcagccatcgtgtgccccacttattcttatagccaacctgaaaagccatcttttataagaaactacctctgctctccatgattggatataattaatcctccttccacatcacctcgccacaaaattgtatctgtgttgatctcatgccacatacctgtatgtattttatattataaatatttgcagacttgtttaatttgccatgttagactaagttccatgaagacagctccatatccattccatttttatatatccacaacatttggtcgggttgatgcttaataaatgtttattgaaggaacaggagtctcccacttctgacataatgaacttatttcccccagtgttaaccctacatctggttcctgtccaagagtctcttcccaaatcattctgattcaactgttcattctgatctcattaaacatttaaatgatatatctaacttcgcttgctttattctatgctcatcctgcagtctcctcataacttggtttcaatgatgcttgcttctagagaaaaaaatgtattaaataagcttatgattcagtcctccagctgtgatggttctcactgaacattagctcagtggttttcgaagtatggtctctagcataacctagaaacttgttagaaatgcaaattcttgggctcaccaagacatactaaatcaaaaattctgacattggggcctagaaatctgtgttttaacaagcctgccagtgcagcctggtcccttttcttctcggagccccactcaaagctttcagtgctcatctcccaccaatgacagggtcctctatggaaaccggcaggacggtttccaactctaactacgttttagagtttgcttcctagggctatccaggcaccaagtatcacaggttagtttcccagggaagcagactctgagacttgcatgcagggagtgtctctggggtgctctcaaccaacaccttcaggaagagaaggaagcagcattgggcagaggcatagtcaaactacagtgctgttggcacagaagactgaagggagtcagagccagggggtagaggtgggcccttagcatccatccttcaccattaggtgtgagttgccccacctccttgatggtgtaacctcagtcccaaggtgggtgggagtgcagcagagcagcccctacaagggccaaaccagagatacaccaggcgccagaagtgctgccagggaatagagaggaaaggatgggcttaaggtaggatccacagaacttggcaatggattagaagacaggatgagaagtgacaggttaacactaacacagaaatgtctaacttcggtagataatggtgccattggctagaagaggaaaccgaaatgaaagcaggttgttcagggagacaaaagttcactgtggacatctcagcagagtgattcagtggggaaaggaatggatgcccagaccacctcagaggaagatctaagctggagccagcaataaagatacaagatgaacaatccctaacgaactgctcctcagccatgctccccagacacgctgcttcagatttatagtccgggtgaggctaggaggtgcgcctccctcagtggaggacagcaaagcaccagtggctccagggagttaaaatcttttgataatttttgttctagcatctgtctgcagagctgtctctcagccattgcctgcctttacacaggagtgcagtccgaaattgggagatgagtgaaatttattatgcctagagatctggatccccagttgtttgggagtatattttctgaaccacttgttggtttaagtaatgcagatttattgatgccacttctcttgaatctgtgactctggacccaccatctaagtgaatgtgcagagggaacggaatggctgcaatagatctccattaaaaccagtgcatcctcccagacacatacagtagtagggaggtgagtcaatgtcaggacagcaccagctcccgcttcggtacatttccaaagttctcagtctgtgtacaaaggtttgctctggggcagcagaaatagccctgggcaggtagtcaaaggcctggtttgatttcctccacttccaggcaagtcactcgaaggctcacaggctttttcctcacctgccacatgggtccagtgagatctactgagctgtaaataatgaaatgagtgtgtgtgcagtcatctataagttgtaaagtactagaaaatggtgaaactttgggatttgggctatttaaggctgaatgctaaaaatgtcaggcattgtggagaaaggaatttaaatataagattgattgactgggatttaaagacaaatgaaggcacacacgcaagtgcacacccacactgacactgcacagctcccgttggaggcatatcctgaccatgcagacctggggctctgcctgtccaagtgcactcctttactacataaaccctccttctcttttggggctgtcaccccaccagagctggcaccgagcccttgctgctgcgcttccctggggtgtcagcttttgacagggtgtttcctccctctgcaggagccttaacatcccttggacttccttccccccacccacccccagcagttttatctcttcctaactcgggaccctttttttcccacacaaagtttattgtcagttgctggtttcatctgtttgagcggctgcaacaaaataccatagactgggtggcatatgcacgacaaaaatttatttctcacaggagaagtcaaagattaatgcaccagcagatctggtgtctgaggggccaccttctggtttgtagatgatgctttctagttaaaacacctatttaacacactattaaacactaagtgtgttaaatagtgcagttgatgtatttgtcatgtcacctttatcatacactaaatccttctttgtctttttttctgtactctaatctctttctgtaagtaatctttgcttgcagcagtaggatatttagagtactgtggcttgacaatatatttagtatttcaagatttccatgaaattcttctgatgtatgagttccctagttaatcttacatatgtatccctttgtaaaaacactttgaacatttaaaatgatacatgaatagtactctaatacaatgccataaaaattataaatcatttgtatagactggtaagtaaagattgtgagattaagaaacgcatcaaaggccattgagctggaaagtggtataatgagaattcaaaccagggtctcttgactcaaaatctaaggatcataccatttctcatgataatatgagtattattgttatctctatcccatagacaaagtgttaacactgaatgagcagtgaaatagtctcagaattttttattttatttagcaattcacttgtcatttctggtcctcagtttattcacgagtaaaataaaatagttggactagataatttctatagtacattcttacacaaaaaatctatgattttgttatttttaatgtgatatactcatggcactcattcacctcattttcccagcctgcctcactggtcattacttctctgtgttctttacaggctccccctcctctacactgccattaaatattgaaacacctcaaagctttacttatgtccacctctcctctgacactatcattctgtctagatgatcccatacatacatgcccattacttcaacctgtatttatacgccaatgattcactatatttccagcctagacattcttttgtactctagttaccagcttgatatccttacatggctgtttcaaaacaactcaaatatattatctctcaaaatcaaactcatgatgtccccacaccatcctagctttccaccaacaatacctatccctattaatagcaataccatttattcagttatccaaatcaaaaacctagaattcatccttaaaattctactatcattccaaatatcctatccatcagcagccactgtattcttaatcccctgtatttccttcaaatccattcacctctctccatatccattgctgcatgactatccaagccatcgcctctaccctagggtaccaaaatagcaacaaacctaatctgttcatttgcattattttttctccaaaactgattatctatatgtagcaagacagattgttctcaaattgcaaatcccactatattatcctcttgcttcaaacacttccatggtttcccattgtttatgataaaaccaaatgcttcaagttcgaagaccggcatgattgggaatttcctgtcaccctagcctacttgctctccatggtacagttgcactggctttctttcattccttaagtacaacctgtttcctcccacctcaggactgtgcatgtgccattcattctgctgaggagcctttttccttccacttcaatcagctaagtctgattcttcctgacaatctcagctcaataagcatttcctctaagaaatgtctctaatatcattaattggctcaggtccctctactgtattgctgcacttttcacagttataattttacttaattatgaatgattatttgattaggtctatttccatccattagacataagcttcatgatggccagattactgttttctatccatcgttgtattccaatacctgacagaaggagggcgggaggtggtggcacacaagagatgctcaaaaacaattgttgaataagtaaatgaatgaggccatttagaaataacgaaagtacctgtttacaaagtacatgtatcaaaactatgaatgcattctacttacatggttttctccaaataaaacaaaagacttcaatcaggattaatacctgggataaactgagtcattaaatctctcctttgccatcaggagtgacattgaaacaaatgtctgcaaacaacaaatacttttttcccaaaatatattgaatggcatttccataaacaaactagaacatgggaggagaaagaaagcaatattaatttaaaattaatcttatcacataacttataccatcagggatttcgggtaaaattcctttcaggcacatccatttaacaagaattgattgttactgaaagcctagaagagaatttggcacatacttggtgttcaaatatttgttgactgagtgaataaatgatgcaagtgtctaagaaacacaaaataaggacatgattacagtcacggtggagttcacagtcatctccaaaatgaggatatgcatcccagggaggaccaacaattcattggagtgctgaaataaaatactcaaaggtcattttacatgtattttttctctaaattacttttcttaagacacagaaaacaaaaaaagaaacttagctttgttactttctaacaaatagttaaatcattaaacaggattgacactagcatccttgtttggtcttatgccttaggggaacatgaaatgtgtgaagacattctgagatctgagggaagggtagacagtaatacagtgggactgaccaggcttcagcacacctttacctcctctcagcagatttcagtgatgagcagtttacaactagattgaaagattatattatctagttctaaaagaaaactaagcctcccaaaagcaacaagggaactgagaggaatcctgcaaaacaaaaacaaattttaaaacttgcactttgtaataaccctaatatgtaatcacagtaatgaacagtaagataatgacagaactgacatatttccttatctattaaagccatattaacaggtaaagcaatgccagtcagtggtacacttcttagaagatatttaatacatactagacacatacacacacacaacattttccttcaaggtgtatgtatcagaaaatcactttttaaggccggatgcagtggctcaggcctgtaatcccagcactttgggaggccgacgtgggcggatcatctgaggtcaggagttcaagaccagcctgcccaacatggcgaaaccccatctctacaaaaatacaaaaattagccagggatgatggtggatgcttgtagtcccagctactcaagaggcagaggcaggagaatcacttgaacctgggaggcagaggttgcagtgagccaagatcacccattgcactccagcctgggcaacagagtgagactctgtctcaaaaaaaaaaaaatcactttttagataaaattcatgctatagagagaagactatgaaaatatgtttagcaatgtgtccatcattaggtgattgagtttccttttgttttgttttactgaaaatcatataaagtatgttatctgtaaaagttctctgacatgcacacataaaaatttgggagaaaagattaactataatgtttaatagattttgtacacatttctttaaaaatatataaaacacaacacctttcaattggtttgcaagaataaccaattgacatcatggaaaatggaaattcacttgctgaattttaacaaaaatttgcatgatgagtgagactgacaacttagtgtcatgatttaatgaattatgccaatggtaaacttcatgcacatggggccaggtaattatgtggaaactttttcaatgcttaaagccaagtattgaaattaaacttagaatcagacctttgaaccattttatgacaatgttcaaaaattataaattctatccacttatattataatattaaaaatatcattacaaaaaaaacctgtgtttattttataactcagcctttttaatttctaatttcataaatatattataatggatattgttagtaatgtagtattattacatgtatataatttataagtaaatatacatgttttggctactcatgcataaaatgtttcacccataggagcacataatcagaaatgtctggagaccattatagtaatagatagatcatattgccacatattttatctcctccttgacaactgagctttccagatcttctggtgaaacgaaagagaaagttgtaacagaagagtgattaaaatgacaaaagcattacttctattacttctattctaataatatgagcaaagctataactatcaagtaataatgcactaaagaaggtgattaatctgatatattcacaggcaactaataagacctttctattgcagccatgaaaaatatgtgacaattatagatatcctgtgtgcagtgtttcaacctttatgtgacctgttctactaacagatttagtgatgttcactttgttagaattttcttacacatgccataacttgcttcagtcttttgattatgaatattatggatattaaggattctagactattctagatttaaaaaataatattgtcacctcaatcagaagggaaatattaaatagttctcattttttcaatgtttactcagtttttgtccaatgtaatgaaagtgtcagcagtacaggttacaaaataaaatgtgtattaaagtaaactcatttgaacaggttaataattgtagagggagggaaaaggctaaaagattgaatgtaaaacttatgaaaagtagatacatcgtctctatgatttgcagtagtcaactgcatacagatgaatcattttaatacacgttaactactttccttttacagatggagaaactgagaggaagaaagtttatatggttcattaaactttgtgatgcaagctaaactaacctgtctctgtattttccatctactgcccttatcactatctcattagaatactcttcaagcatctccttactgattttcttaccaagcatttgttaagttctaatgagagttggtagtaacattttcacccactctgtgaaatatgaaatcttattcataggcctcttcttttattcttgtatttgcatatcaaccaattaatcaacttgctttctttatgttgcttattatcttagtccttactaaattgcctcttaatgttgtccacataacagaaatgttaaggtggatacttaacattttagtccagtctagccggtgccagtgcaatgccaaatcatgaattaaaatataattacaagaaccacttatcaaattttaacaattccttcagctttgtgacagttttttctacttcgattaaagtcaagtaaaattaaagttaaatatttttattaaaatatctcctttaacattccatattaataaacatattaaagctcatgcttctaagtagattactagaagttactttatcgaattacagcaatggttaattctagatcatagaatttagaatgactttttgccttcttcttttttttccttttttttaaacagagtcttgctctgttgtccaggctggagtgtactggcgcgatcttgactcactgcgacctctgccctgcaggttcaagtgattctcctgccccagcctcttaagtagttgggattacaggtgcctgccaccacacctggctaattttttttttgtatttttaggagagacagggtttcaccatgttggccagactggtctcgaactcctgacctcaagtgatccacttgcctcagcctcccaaagtgctgggattacaggtgtgagccactgtgcctggcctgactttttgctttcttcttaatacttactagtatttcttgaatttttaaaaaagaaacataaagtactttgataaaaccaacagtctcattgttcttaaaattgttcaaaggttctctggaaaaaaaaaagaaaattatcatttggttaagaatcatgttggtctgacatcaatcatcctataggagtgaatattgaaaaagtaagatatattgtggtataatcgagattgcataaattttaccatttttgagaagaatctgctccaaatcctggcttaatgtaatatccagcatgctacttaattttcttgtcttcaccttttcatatccacatccacctaggtgccacctcacagtataagccagcataatccattcttctcaatgaaaccacaatacatctgaccctgcatctcaggagaactgtatcagccacagcacttccagttgactatgaatctgaatgttatgcctcaggagaaacatccttgctgggactgagtagtgattcaaggagatagttatgattcagtcaagaaattaataattagtgttatttttattattgagacagagtctcgttctgtagcccaggctggagtacagtggcatgatctcggctcactgcaacctctacctccccggttcaagtgattctcctgcctcagcctcccaaataactgggacagcaggcacttgccaccacgcctagctaattttttgtatttttagtagagacggagtttcaccgtgttagccaggatggtctcgatctcctgacctcaaggtccacctgcctcagcctcccaaagtgctgggattacaggcgtgagccactgcgcccggccataaattattaactgagccaggcacagtggtacacacttatagtcccagatactcaggagactgaggttggagtatccttttttatgttattttatttttaattattatgggtacataataggtgtacatacccatggagtacaagtcatgttctgatacagacacataatgtttaataatcacatcagggtaattgggatatccatcacctcaagcatttatctttctttgtgttaggaacattccacctccactcttggaataggcaccctgttgtgctattaaatacgaggtcttattcatttcatctaactatatttttctacccattaaccatcacctcttttcccctcttccccactacctttcctgtgaggctgcaggattcttaagcacaacagttagaggccagcctggacaacatagtgagactcaatttctaaaaaataaaaaagaaattaccaactaatgctaaaaaaatagtctctgatgcttaggtatgaattagaaatgaccaaaaaaaaaaaaaaaaaaaagactgccctttgcttccttctccccttctcttcaagttttccattgctactcattttagtctggtttaatcaggtttcatccattaaaagcaattgttgggatcacacattttgagttgtgtcagtggacttccctcatgctggcatgattcctgccccaagcccttagtaaaagccaccaagccatataacataatctctcattgagtaaaacatctgatgtgtttagaatgacttctagcaaaaaaccagcctgtccagcatcatctctgtataacagataaaggaataggtactgcatcaaaaggttatagaacctgcccaaatcaatcccatgtgttttgcaatggaattaggttgaactaaagtgaaaattcagttttctactcctcattaacatgtctcatgttgcaaggttgagaggaaggagaagaagaactgtatttacagagagattccccctctctttctttctacagattactaaaacattcaaagaatcaaatttaagaaatcagttcatcagagctcatgttgccaaactgaggtgagtggaactgtagaaaaaatatttaagtatagatacaatgtggcatacttgactttttgtcacagaatgaatagtaaatgacatgttcagataagttgttgtaatattatgaaaatagtattttagtcagcttaaaaaccaatgccaaaaaagccaaacatatgatctatttagctactaatgtaaataaccatattatatctattcttattgggaagaggaagaaggggtggagagagagttggggtgaaggtacagtaacaaggccatcctattgtaaaactccagtggatatcattcacagtgcagcctatgtaaacagtccctcctggagttgtacaatgctgtggtttgggtgtatccatccaagatcaagacactatgaccaacatcaaaagtggctttttggttttatctgcctgatgtgctataataaaagggtattatggccaaatccaaggcatgtctatcatgaattaataataggaggagtagcagcatgcatgctagttatttgccattcctgccttagttaaatatgatgtgataaaaccagcctttccaactgaaatagtcacctttactgactctcccgcaaatgtctcaaatgaccacattgctctagtctttaaataatatgcaatagttctttggtagaagaggaattatactaattctttctcaaatactagcatcacaagaaaattaattcttgttctctggagagtcacctagtaagtatctggagcacagatgtctggtcaggtaagttttgatgaggagttaaagggataagaagagtccatgagaagggtattttccaaaacacctttcggtcaattcagtgcacattcacttagtactttcttgtcagtatctgtatcagccactaatgttcaaaagtgagtaagccctgaaaacctgtaggactacatgagccttctgccttttctctccttttgttcacttcccacttatcactcaatcctctgcaacctggcttcaataccaccataaaatatcaactgctcttgccgattcaacaatgacatccagataacaaaatccaaagaaaccacatcagtcctattcttggacctttcaacagtatttggtcctgttggcctgtcactccttgaaataggactatcccttggtttgcatggccttgtataccctgattttccccttacctccctagctattccttcttagtttcctttactaggtcttacttctttgtatattccttaaatgttgctgaacatcaggctgtgctctaggcctctcatcttctcaggtcacactctctcctttccttggccttcactgccacccatatgctgagtgctctcaaagttgtatctctaggccagtcctcttttgcctccaaacatgaatatatgcagccatctacttggtaccatcacatggataattctcatgatctcttccagtatgactgcttctttatttttttctgggctcttttttagcattgctttacatggaactttatcatgtctctcaacctctattttatcttttatctatgtatgtagagtctgtgtaatttcttcatctcttttagataactaatatctcttcagctttgacttgtattctgtgtaacccatttattgcgttttcaatttcaatgagtatgttttcctatctgcaagttctatttgtttcttttgagaatcttcctggtcttttaaacacatttcttattttaatttttgggggtacctagtagttgtatgtatttttggagtacatgagatgttttgatacaagcaaacaatgcataataatcacattgtgtaaaatggggtatccatcccctcaagcatttatcctttgtgttacaaacaatccaattatattcttttagttatttttaaatgtacaattaaattattattgaccatagtgactctgttgtgctatcagatactaggtgatcttttaaaaataatgttttctacttaatctcatttttatgattccctcttttacgtcatttgtcatttcaaatacagtcacttgtctgttgattctattatgtgaagtttttgaggataatctttttgttactttgattccaccttggtatggtttggctgtgcccccactaaaatctcatcttgaactctggttcccataatacccacatgttgtgggagggaccttgtgggaggtgattagattatagggacgtttcccccctttgctctgttctttttcctgccaccatgtaagaaagatgtgtttgcttccccttctgccatgattgtaaatttcctgaggcctccgcagccatgcaggacctcttttctttgtaaattacccagtctccggcggttctttatagctccgtgagaaaaaactaatacacacctcatgatgtattgtttaccactgaaattgtatgcttaaatttaatctcacttgggaccctgtacaacctagacttaacatatctacctccagagcagttacatctgtcagacattctagaggaatcagcagcacatggactttgttgttgttaatttgttgtcgggggaggggggagggatagcattaggagatacacctaatgctaaatgacgagttaatgggtgcagcacaccaacatggcacatgtatacatatgtaacaaacctgcacgttgtgcacatataccctaaaacttaaagtataataataataaaattaaaaaaaaaaaggttctgggagtattcaggtagtattaatgaagattcagacatcgtgcagccaggcccatgcttatgaattttcaggtgatacttctttttcttttttcttaatttaaagctggatctcggaaacagataaatttatttttttatgacatgacgagcatttttttcattctagttcatgctgttattgggtgtttagttctttgagactcctggcctttttctaaaacctcaagttcaacttcctattttgcactggcccaaggtcccatctccagtctctatgtaaatgctaaacataagcctgtggaatattctagtctcaccacatactattcacattcttctttgtttttggtcttccaggattttccttacttttctatgaacccagtcttgcatttgaaatggaatttattatatattatctatcctttctatttgttttatgcagaaagtgttttctaaaattatttaggcttccatattgctagacatggaagttgtaattatttgttcagtgcctgtttctacatctaaactgcaagacccatatggcaactgtgaatcttagtcccagctaatttctgaagcttagaatagtgcctagcacaagaagttgtttatctaacatttttaaaaataaatattaaattcatatctggaatgaatattaagttagagctggtcattgaggtgagaggaggaagccaagagagaatatgagagcctcaaagccaaatatctttaatgtactttttcagaaaagaagacagccaatgtcaggtggaggaactggtttatgaggtaactttcctggaagaaaatagaaattactgaggttttagataatccaaatatttaatcaagtcaccaaggtttattgtggggaatctttattattaattaaaatgagtgatgaaatcttaatatacgacaaaagttaaaatttgcttttgcaggcagatgaatggtctaggtatcaaaaaattaagttgagtctctaactcacacaaatttacaaccctatcactttatgaatttgtttaggagattatttttaataacactggtgaagtctaagaatagctaaaatttatagtacacttattgtgtgctattgactcttctttgaagttttgcatatagtgattcatctaatcttcataacccattttacatgtgaagaaacttagatatagaaagattaagaaacttacataacttatccaaagttacacagtaaaactctggcattataacttcaaaatcagctatcctacagtgagtacagtgttctgtgcattgaaatcaaataagtgagatagcatcgtgatatagtattacgtatgcaaacactgttacagagatctgtctaaagttaaattccacaaatgaattctttaaaagggtttaatcaagaagaatatataaacaggatggtgaaaaattgtcatattatttgttttttaaaatatctttatgatttacaggcaagatggtagtggtgtgagagcggatgttgtcatgaaatttcaattcactagaaataacaatggagcatcaatgaaaagcagaattgagtctgttttacgacaaatgctgaataactctggaaacctggaaataaacccttcaactgagataacatgtaagtataatttttcataaacaattttatttcaatatatccctcaagtttaccaattcaaattcatattttaattgagaggctgacttttctttctttgaaactaaactgtgaaaacaatccattaaaaagctaaatataccatatagctccctaacgtaaatcattctaagacttaaagaatcatttggcatttatatagtaaattttatttgctaaaaattctcattaattatccctgcaacattccttatgagtgatgttactgtcagatgtcattagtggataggccataggaggggtacatagatgctcaaggtcagagaactatttaattaatgatccacctcagaggcttcttcatttttctttgtaacatttatcacaattgaaattacaaagttatctgtgtaaattttgtattgtttggcttcatcctacactgtaatcatcctaaaagaaagaaccagtcaaccttcttcatcctactaccctcctaccacccagtctccatcatataacacatattcaataaataattcttgcatgactgaaagaaaagaaataatatatgcatagaatttaaggacattcctccaagttggttacattctgctagtttaataagccattatttcttctcgatgagctcaagattaaaaggattttgatgattcccatactagactggtaggtaccagttacagatgtactaactgttaaatattgaaatgctttcctatttgttggtaaacaattactgcatcaggcccacaaagttgtcttccgagatgtttcaaatccactgcccctgctgctaaagagttatgcttagcaaagcaaagcactctaagacactgctccaactccatggcctgattgcatcttttatgactggccaatgctcacgcactgcagtttgttaggtagttgaatattacctctgcttccacacattaaggaatgctcccgaacgcacttcccaagtgtttatttatttatcattatactagacaatatggtgatacgatggtcacagaatagcggtttccacctccagagcccataatctagttgaagggaaagatattccaacacaagagtgttgacaatcaagatagaatatgatcaagggcccagtgtgaggcccaggcaatgatcactgcaggaatctggggaagaaagagaccagcgtgcttgggatatctagcaaaagtttcatgaaggagaatggactttgactttgaaatatgggtaggatttacatattttgagatgagaaaaagaaagttcccagagaaggaaagcatgaaaaggcaaacagtctgtactgaacgcgatgctttgacagaataatgaagaaagggacctgctggaatgattgatcagtgttcatcattcacaccatcatcatcaaaacacttatttaatgagaacttactgttttttaggcatggctttaatgccctatatgaatttttttcttgattaatccttacaacaaacatatcccatagatagttttattgtcccccttagaaaagataaattgcctaggctgacacagtcagtatatgaggcagtcaggattcaaactaagtctgtttgttcaaaaaattaagaatggccagctttttaaaattttctgtctccagaagtatgatttggctccactgaagtttgcaaaacaaatgtgatacccaaaccttgtgaaacttttagtgggaaataactttgcataagtcggtttgagagagcgtggaaacctgtcttgaaaagttttaatttaacttgcaggaaataaaaatgatgggtttctcaattaaaaatttcaatcaaggaaggatatgagctaacataacatttttttaaaaagatcagtctggtaaggtagaggtgcataaactgaaaaggagcaaaagtggtggaattcagttagaaaattattgtaactgtactgatgtcaaatgatgaaaccatgaactaaagtagtaccaaaaggagtgaggaggatggaataattcaaaagatagaggacagatgtgcagaacctggagattataagatgtgaaaggaggagtttgagaaaatttcagattttggaagtggtgtcattttactaaaaggatataataagtagcaaattttggataaagttgggtcccactgagtttgagatggctgttggacatgcagagaaaactgtcttgtatgctgttcttaaattgaaatagacagacctttaccctctgatactgacatattttcctttccaggctcaccctccatttccctaaacacaacacatgcactagctctccttactttattgctccacaaacatcttacacctccaagcatttgtgcccactgtaccttctatctggaatctcttttgtcctcttgtgtgcctgaaaaattcctttcagatcttcaaaatacagtgcagatgctatttcttctagctcaaatattatctcctccatataatttaattactctcttttttcttttctctactttgcacttacatttatttgaatgattgcttgattaatttctacctgtaaattatgtgagggcaggtcctctatattttgctcgcagttaaatctgcagcacttattatagagtggtatcattagagtaatatacatatatttgaggacatgataaattaacttcccctatagtatttatcacattgcatctcaatgacttgcttatgtttctgttttcccatataaattgagtaacttgaaaaaagagatatctattaagtatttaatgagaaattaaagtacaaactttagtatgcataacaacaaattgggaaaaggttgtaaacaaagagatttgtagggcccatgagttagagatcgtttcagcaggtctgaaaggaagcctaggaatctgcattttagaggaccacctcccaaccccaacaagtaattctgcttcttgttgtctgggtactgtactttaagaaattatggtgaaatgatatcagcctttattgtatttatcttattctcattttttaatactagcacttactgaccaggctgcagcaaattggcttattaatggtaagttttaatattattttgtaactgtaatttgccaaatcataaagagtaaaagtgcaagtcttttgtgtacttttggccaaggcagtatctatcaagttgatgtctttgttcttagttcgctcaggtggtgttgaaacaagacagtgctgatcccaagtgtcccatggagtggactttaggtttcccctttccttttagaaaaaggaagaagttgtagtggaggactacccactctgcactcaaaattgccctcatgaaaatttctttggcagctttgagaaccttttactgccctggttctaaggtggcatttctgtagacttacaaattatgtttgatgacaccgtttatgtagcttctcctaaccaccagagtagcttgctttgttgtgaattcaggttaatcacaaagtataataaaaaagaattgtcagaagtcttcccagctttgggtctataacctgaaggaaaagtcactactcttcaacatcatcctatgtactctcaggctaggatagcagaaatgcaatccctagaaaacagcaacttacttctctgaccaaaaaaatgcagttaaaaattagttcaatgtacctggtagctggcctatcttaggtacttcagtgattttacaaagtgatggtagtcctatgggtgtttttcagcttcactacgtatttaattcatgcttattgttaatgaaactgtgataagcaatttactagggtatttgtttgggagatgccacaaaggaacacatgtatctcttaatggaagcctggtcctcctttatccaggaaatttgctaggaaaaaaaagcctttaggtggttgtgctattaaaccagggcactacttaaaagccagcccagcaatagttgtgtgatttaccattaatttcttagtaatagaccacacaaaagaagaaaattatgggaatgcgagttgagaggaattgggtgatcagcctaccccagcccgtttcagctctggccagtagactattcacgagctctttgaaaacatttaaataaaccttatttagatactagaaaccctctgtcaccctcaagaatattctgtggtatagcgactcctttatgagggcatgtttggtaatacagcatcagtcttggaggtggactggattctacaaggtgaactgcagtcactaaggagtcttttggatgagaccagttttcctccaacttcaatgtgtgcatgaacctcacatcaaaatgtagctttagatttgtcccatgatgtggttccaagaatcagcacttctaataagtttccaggggatgcccatgctgcaggcccacaaaccacactgagcatagcaagactattgagaaaaaggaaatttcccaggagtctgtggcctgagctggcacatccaataatgacctatcttaacctcaactcatgaggaattccagggaactctgaagctgctcaaaatttgaagcctatatgccaactaaattcagaaatgttctccaaaatgctatctataagcaacagtagtcacaaatgcattgtagaaatatatcgatcatgctttttggaaaatccagcatgtcctgaggaagaatgtataagacataaaagtcataaattatggaaagactcttcagcttcttccaaatgtaaaggaatcatgatcttcccagcacattaatgccctttctcattagaatgtggggccggtccagacctaataacattgtctgagcagagaatccttggaggcactgaggctgaggagggaagctggccgtggcaagtcagtctgcggctcaataatgcccaccactgtggaggcagcctgatcaataacatgtggatcctgacagcagctcactgcttcagaaggtgaggccaccactacctacccatctgggaacaattagaatagacaggtcatgaagactgcaccctctaccctaggattgaattgagccagaaataattcaatgcaaaaaaatcagtaagaattttcttcctattcatgaaaggaaaaggatttttcccctttagcatgctaatttagtgctatttctctgtttcaggtaataatatattagcacagtaaagaacaaagatttatatgtcagaatgttttttaaatcctagctataaaagcttaagaaatttactaaatctccataagctttattttttttccaaattaagggacaacactgttatctgtgacttagtgttactggtagcattgagtacactaatgtaaacatacgttaaatgttagcgaaacgaattgctgtggaagatttgcacattatatcatgggagctgatggctaacctagagactgccccatgccattaatttattcattcataaagattattgagtatctagtatgagcacagtgttatatattgtagaagctactagtataaacaaagtattgcctctgccttcaaagagcttacactcgaatgttggaatcagaatgcacaaaaataatgatcaattacaatgagtagcataaataaaattaatgtaggcaacttacaagaattcttaattgaggtgactaaactattgccaacactagggtgatatgctaccagtggcgagtaggttgcataaacttaccttattggtaaaaagaaaagttcacattgctcataaaagaaggattttagatttcagcataactaaaatctgtttcaaacctgccttgttactggggcatcgcagaccacaacagttgttgggaacttaactcaaaaagttcacccagaaaaataatggagatttgaactcgtgtgcccctgaccatatcaattttcttctcagactcttactctaaactggacctccttatcacacacacaaagccttccataggcagatcaatccagtcttatttctcaaagcatgtaccttgagcttcagataaacagcattgttctcttcccctggactcttcctacatttccctacctatgagtatctgatcaatctgcttatccttgaaatgttaatatatttaccacatctctatttgaattttatgaaatttttgataatttctaagtagttttttcagatttataggcactacttcatggtacagtgactgttacaaacgtatttgttaaatttagaaggaataaagatttaaaagactagggtagttactgaactaaagttttaggaaatcccaaattatttcaaatttttcttatggtaattttatgacttaatatttttatatgcagtgaacaaatttgaaactttaaaagatactcccagaattatcagttttctgatgtagattggcaaatttattactatatcccaaataacccaagagacaaaattcacaaaaacatttcaattttcattgccacttgaaaggccaaaaagcagaaatggcacgcattgatttcaatcgtactcttgagtgtgggaaccaggaattaaaatacctggacttatcaggcacttagcataaccaagaacggaatagaaacctccctggattctaagccctattcagtcccaatcaccaaaaaccaagtaaacgatatcactataatgaaagccacagttataaatatcgacaacgattaccaaaggaatccatggaactttgaattttgccaccccacatccttctattcattaccatgattgatccactaaagctaacagactctgtgaaccttgtattggacccctccctaaagacctgattgtcactgagaaccatcagtgaggatttgtttggggcatgaccagccttacatcaaagtacatagaagtgatgaggtcttatcaaagaggattattgaattatcacctcttctatgtagctttccctgatactctctttcctctccattgagttccacagaaatttttttatctgcctttaacagttgtcctcatgatttgtgatatttgacttacctcttgtcagtttccttcactagtgtagagttcctcaaagaaagagaccataattacttatatttttattcctggagactcatactattccttatacaaagtagacacttaacaatggcttgttgaactataattaatgaaaataatagctaccttcatgaaagttcactttgtgccaaacactatagttgacataatacatttgtctcattaatacttaacaattgtgtgagaaggtatcaccaatcacattttatatgtaaataaaccccagagctattaattaacttgtcataaataacacttttcatatgtggcatagccaagatttaaatataaatgttactggttccaaaatgatgctctaattcacttgctggaaagaaggaaaggaagaaaataaacgagtggaaggaagagagggagggaagagagaaaaggaaggaaagaaaaaagagtctcttcagaaccttcactgtaaagactccgagcaaaagaagttgaatataaaaacaacataggtttgtttgttttctaatattttttcttcaaaatttttaactcaggttcactcttacacaaactactgtgtcttataaaagtatttccggtcatagaatttttattttctgtattaactccactatctaatctccataaaactcctaaattggtattatcggtaacattttgtttttactcaacccttaggaacaatgttaagttaatcagccctccacatcacagatccttattttcatcagtctgtacaaggcatttctctcattttaattttttttcctcctgtcatccctggatttcactttcactgccctccttccacccatatgcctcatactaatatattcgaaatatacatgtcttaaaggtacatgcacgcacctacaaaacctatagtgtttttttgtatgtatatgtctttaatttaaataagtagcattgtgtaaaagtctaatattgtttcttactgttttcactcaattcttggaattttcatctgatgcactgctgcatagcaccccatggtatgcagccaccatatttccttcatccaattaggttgcatgacctaccttcccattgccacaaagagtacacacaaaatatttgtacttatctttctgtaaaccttcaggaatttcagaagcacacatgcaggctgctaaatataccagaatactttccagccacttaaatctttaccagtattgcaaaagaggccccatttccctccacatcaacatttagtattattcttttgtttaagttttatcaatcttttaaatgtacacaagatgctcatttttataattttaatttctcagattactagtttgagtatcttttcatatatctaagagctgttttgatctcccctaccatgaactgccactaatattctttgcctattttacaatggtttttctgcttatttattactggtttacagacttttaaaatatattctacaaaaattttagacattaaacattaccaatattttcccatggttcctcatccatctggtaaacttgtctatggtatatctaattttgatttaatagaattcattctatttttaccttttagtttgtgtttttgttgtttagccaaaaagtccccattcctaggtcataaaggtaatgtcctttttttttttttaacgctactgttctctctctgtctccccctatgtatataggtgcacatatacttgtacacacatacatatacctatatatgaggggagttcgataagtttatggaaaataaaattaaaagataaaataaaaaattataaactttatttctcaacataagctccttcaagttcaagacacttttgtaagcaataataccagccatatcgtccatccctaaagaactgagggtcctgagaatttaactatgtcaatgcagtcttttttacattacttttttacagtacttattgatgaaaaatgggtgccttttaaagattgttttaagattagggaacaaaaataagtcagaggaagtcaaatcaggactgaaaggtggatgcctagtgatttattgctgaaactttcataaaactaaccttatttgatgagaggaatgagcatgagcatggttgtgatggagaagaactctggtggagctttcctggacactttttctactaaagctttggctaactttcttactctcataagaagaagatgttatttttcactgaccctttagaaggtcaacaagcaaaatgccttcagcatcccaaatgtctgttgtcatgacttttgttcttgactagtctggttttgctttgactggaccacttctacctctttatagccattgctttgatggtgctttgtcttcaagattgtattagtaaagccatatttcatcttctgttacaattcttcaaagaaatacttcagaatcttgatctgacatgtttaaaatttctattggaagctctgaccttgggtgcagctgatctgggcgaaacagttttggcatccatcaagtagaaagtttgctcaactttagtttttcagtcagaattgtataagctgaaccagttgagatgtctatggtgttgtctattgtttctcacagttaattgttggtcctctttgagacatgaacaagatgaaatttttcctagcaaactgatgtggatgatctgttgctgcgggcttcaccctcaacaacatctctttctttcttgaaacaaattatccattagtaaactgatgattgggggagatgctgtccccataaactttttgtaaggcataaataatttcaccattcttccagtttcaccataaatttgacgtttttttgcttcaattttagcagcattcatgttgctttgataagagctcttttcaaattcatgtcttattcctcttagtgcctcaaactagatcttgttcagtatgacaagttagtatgagtttatctgcatgcaaaaatctttgaaatccatgcatagtttgtttataatatacattttcaatgaacttttgaagaccccatacatacatatgtatatatatgcacacacacacacacacacacaccaaaatcttcaaccattatcagacttagtgcagaaaaattattcatccattaacaagataagaatgccccttatcatcactactatttaaatggagctcctggctaaaggaaaagacagggattgaaaaaaattagttaaatctaaaatgtttattatttcaggtttcttagttgcttaaatgggaagggaggtatggacaaaagagaaatcaaagatatttgtgttatgctacttatcattaaagtatcagaataacttcattggaatagaaaaacaccaagatcaccccacgatatgttttctaaaatcttctccatttctttagacaagtgaccatgtattcggccagtgaagaattaaactcacttgccagcttataatgcaggaaaatatagcaaagagatgtggatccaatagtttctagatagtggtacaggatggctaagatgaatttatatatctgaaatgttcacaaattccctactcatatagcatgttttcataatgttttagcaactctaatcctcgtgactggattgccacgtctggtatttccacaacatttcctaaactaagaatgagagtaagaaatattttaattcataacaattataaatctgcaactcatgaaaatgacattgcacttgtgagacttgagaacagtgtcacctttaccaaagatatccatagtgtgtgtctcccagctgctacccagaatattccacctggctctactgcttatgtaacaggatggggcgctcaagaatatgctggtaagtgtctcggaaaaaaaaattaacaatagaaatgtcttatatttgctattaggtaattttttaaattaggaaacatctggaataggtgtttctattcttctacagacagaaccattctatattctgctcagcccaagctctggctacccctgagtctccttagcaaagcaaagcaatgctccagaaactatgggaattctcaaatatagtaataggaaaatgtaaaagaaagttatgaagacacgagttctttaataatccagagattctataagattcaaatagcttccctataaacaataaaaaagattttgtttgtttgtttgtttgcttgttttttagagacaaagactttctcagactggagtgcagtggtgcaatcatggcttactgcagcctcaaactctggtcttaagaaatcctcttgcttcagcctcccaagtagctagaattataaataagtgtgtaccaccatacccagctttttttttttttttctacagacaggttcttgctctgttgcccaggctggtctggaattcctgccctcaagccatcctcctgccttgttggcctcccaaagcaatgggaggatttagattagacattgtatgagggcttaataatccttaaggtattaactgccctttaaagtattctgggatatggcaaaaactcgatgtgtatataaacattggtcatatttgtttattgaatgaataaaatggaaactaaaatgaggacaatgcacaagagctactagaaccagtaagagtatcagcgaaggagtggaagggtagcattgacaatttccctgggcttttacccatgttgtagattgtctctccaaggaataatacaaagccttaatagtcctagaacacattctattgtgttcttatggcccaaagtaaattggtgtagtagataacatttgcaccagtcatgaaaaactattggtgtcattctgagagtacatcaatataaaatagactagttctttagccttgaaactagactggtttctcttttgctgctaggttaaaggttattcaatatgtaatcttccaatccaaaatctgtcagtggataatttaaaagcttttagtcaattttaagatatttgttttcttaaaattttaaggggcactgtgtcacaaagctaaagaaaaaaaagaaaaaaaaactgatctgtgaaaggggttatcctcatctacttggggaattttggctgcgaagaaactccaaagtaaatctttagaagccttcattgttaaatatgaaataatgtttggagtacatttatttcttctcaaatttattatagggtcaataatgtacacatcttgaagtccatttttttcctgcttttataacaaacaggccacacagttccagagctaaggcaaggacaggtcagaataataagtaatgatgtatgtaatgcaccacatagttataatggagccatcttgtctggaatgctgtgtgctggagtacctcaaggtggagtggacgcatgtcaggtaagctcaagacaatctcatccatgtcatcatccaagaagtgtataagcacttcctagtatgtgataatgtgatagacataagtgtaacagttacaatacacagccctgttcctctaaaatttataatctagattttagaaataaatttttttatgaatgaagtttatctatcatgaaagcattaactctgagaggccaaattacagagtagttaaccatccaaagctcaagaatcagaaagacctcgatttgaattccttaacctctattaccaagtctctaactaaaagctggggataatcataatagcacctaactttttgggtactaagaaaagttaaatgaagactaaatatatcaggcacatggtaaacaacaaagaaatctcatctatttcactattattaatgtagaccatggtcactcgtgttaataactttaacctcaaccttttaactgctgtgaaggattaaataaaaaattaatcactatattataaaaattaattgatatataataaatgaattttaagagatacgtaataattcatggactccttgaagatagaaaatttatacaaaatcctagtaatttgagtcacaaaagctcctacaataatgaaacagtatgaatgaaaaagaaaagaaataactattatatttggatctagcccataatttttaaccaaatgcacaaaaacaaacaacaaatatgaaattctcactgtaaagtgattaaaatcaaatttgaattctaaaattttaaattaaattatctaaacataattgatgcagttatatgttttaataggttttgttcacatatctgaaatccaactccacacagtagcaggaacagctggtgtcagaaattaaatattcttttagtctggagttttaaaaaatcaatctgtttacttgagtaatttgttgctgttttcatgggtgaattgtatacagaaggataagaattattcttcgcatcaaaaggtcactgactttcatatttagtgctcatggtctttaaaaagtggataaaaagtagttctcacatttcatggaaagcccccaatccatgagcacatttcccaaaatgaaacatttttatcaactgcaagttgtgtgtaggtggagatttgtttttcaattgtcaagatactgttaattacccagtcctttatctccttttggtggagatgtctctgtgctaggaaacccttcttgctctccttcctgtttctcttttactactggccctgaaacaacaaattctcaagtttcatgacagctttccaaagaatccatcaatcaaataagcaacacaactcgacactgacaattccagacctactaagagcattaattaagacttaaaaataaacatgagttttaaaagggtgttattcattattttcccatttataacgtcccttaccttctgtccttcagtgcatacaaattattatcttccttgaagcccagttcaagccgtacctcaccatgataccttccatgtatattccactctaggcctcactgatttttaactgaaatactataatgcatagttcacacttaaaaaaaaaaaaaaaacacagcactttacataagagcttacaggatcctatttgttttatccattcttttgttcatttttacaatcattaattcaaaggaattatattaattactttctatgcacccgacgttgtgttaacacaacaatactatccctgcattcagcaagtctatggtctacaagagaggacacaaattcaaatgtctgtagtcaagcagtgaagctggctagatatggaaaaattacaagtccctcttgctttaacatttgcttgcccacatttggtcagacatcatgcaaaataatttctcactatagaaaaaaaaacactacaaaaacaataatataaagaactgagaactggttaactgaagcatgcatatgtcatctaaaagaagcaggtgacgaccagcttcatgaagtacttgccatgcatattggcacttcacacactgacccttctccccacctagaccagtaattaaacaggtatggatgagctagctactaagagcagccaactgaatagctgactaacttagaagcacacttggtaataatagctgacttttattagtactgactatactatatgctaagctgtactcaaagtgctttgagttttaaactgatacaaacattatatgaggaaacagaggtacagagagctattcaccagcttaccaaaggtcacatagctggtaagtggaggacttaaacccagactatctagtttcagaacccacagacttaatccatcgtgcagaacataagacatactccatctgtctccccaactaggttattatgtgcacaaatatttattggttggttggttcattattatgactgggtggtaagtatgtcattaggagtgttttgcttatgactatataaatttcttcaccaaaagaagactttctgatgatatactatgcatcagacaccacgcagggtgctaaggttaggaagataagtgagacttctagaaactcattcattcaacaaatatctcctaagggctagaagcttaggtttcagcagtgaacagaataggtatgttctctttcgtgttggaccttatagtatatctgggaaaacagacattgaataaatatcacaaatgcaagtgagtgtttcagagacatgcagctgctacatcaaaacaaaacagaacaaaacaaacaaacaaaaactgaccagtgggattaagtgtaaataggcacacaaatgcacaaatatgcttttataaaatagtgaagcagtgacagagacacacacaagatataaagacacaatgaagaacaattgagcccaaagctggaaagggtgagagtgtgaaggaaaaaggttgatcagagaagttttcccgaaggagagaaagcctggatgattaggaggcaaccactcggtgactgagggaaatctgaaaaatgtatttgtcatcttctcagacttgctgaaggaatgacttgggtactttgaggatttcagtaatttttccatgacttggtataatatttcaaaaggaaataggctgactttatttgtataatgaatgtgactccttcctcgactgccatagaaataaactccttaatattttgggtttgtctttgcacttaagtaatcagtcattctgtttttttacagggtgactctggtggcccactagtacaagaagactcacggcggctttggtttattgtggggatagtaagctggggagatcagtgtggcctgccggataagccaggagtgtatactcgagtgacagcctaccttgactggattaggcaacaaactgggatctagtgcaacaagtgcatccctgttgcaaagtctgtatgcaggtgtgcctgtcttaaattccaaagctttacatttcaactgaaaaagaaactagaaatgtcctaatttaacatcttgttacataaatatggtttaacaaacactgtttaacctttctttattattaaaggttttctattttctccagagaactatatgaatgttgcatagtactgtggctgtgtaacagaagaaacacactaaactaattacaaagttaacaatttcattacagttgtgctaaatgcccgtagtgagaagaacaggaaccttgagcatgtatagtagaggaacctgcacaggtctgatgggtcagaggggtcttctctgggtttcactgaggatgagaagtaagcaaactgtggaaacatgcaaaggaaaaagtgatagaataatattcaagacaaaaagaacagtatgaggcaagagaaataatatgtatttaaaatttttggttactcaatatcttatacttagtatgagtcctaaaattaaaaatgtgaaactgttgtactatacgtataacctaaccttaattattctgtaagaacatgcttccataggaaatagtggataattttcagctatttaaggcaaaagctaaaatagttcactcctcaactgagacccaaagaattatagatatttttcatgatgacccatgaaaaatatcactcatctacataaaggagagactatatctattttatagagaagctaagaaatatacctacacaaacttgtcaggtgctttacaactacatagtactttttaacaacaaaataataattttaagaatgaaaaatttaatcatcgggaagaacgtcccactacagacttcctatcactggcagttatatttttgagcgtaaaagggtcgtcaaacgctaaatctaagtaacgaattgaaagtttaaagagggggaagagttggtttgcaaaggaaaagtttaaatagcttaatatcaatagaatgatcctgaagacagaaaaaactttgtcactcttcctctctcattttctttctctctctctccccttctcatacacatgcctcccccaccaaagaatataatgtaaattaaatccactaaaatgtaatggcatgaaaatctctgtagtctgaatcactaatattcctgagtttttatgagctcctagtacagctaaagtttgcctatgcatgatcatctatgcgtcagagcttcctccttctacaagctaactccctgcatctgggcatcaggactgctccatacatttgctgaaaacttcttgtatttcctgatgtaaaattgtgcaaacacctacaataaagccatctacttttagggaaagggagttgaaaatgcaaccaactcttggcgaactgtacaaacaaatctttgctatactttatttcaaataaattctttttaaaataatttccctgcctaattatttatggaagttatgacttttgaaggacaattcaaaaccatttatttaattggttctgcaatgaaagaactgccccatatactctactaaaggcttggcactttctgctgccttttaatccagcgctataattgaggcaagcgtccagcttgacacctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 20 - DNA, 34073 bp, synthetic construct
  misc_feature 1..34073: note = Recombinant polynucleotide
  source 1..34073: mol_type = other DNA
gagggagggtggtgctttgctaatggtgaattactaactcctcaataaagaatattatttgaaataatttttgaaatttcataattactttgggttctttcttaatgataaataaataatagtatattacaaacatacattaatatttcctgaatgaatacaccacaaatctcccttaaaatatagcaagaataaaaattatactatttctgacaatttttaatttctcaaataataataccactctgatttttaaacatctacaccactctggctttgccaatctttttaaaaattgaaaagataataattttatcataattacactgaagcatagaactttttctttcaaggaaagcaaatttttgaaattctataatataacctcccataatcctgaataaattaaaggttcaacaacttagtaaagtaagactgaccttcccttttatttctttttcagatcaaaaatcttacttttataggagcagttttcaactcctaaatgttgaatataatagtcagttaaattcaccagctacacaggaatacaggactttgagtggaagaattgaatctctggtaagttaatatttgtctttgctctttattccattataaaatgaatatgataataaacctaatgttttgtaatatattttcagttgctaagtgctctacatattttccttccttgaatggtgaaacatgtgtttctctctgcttttatccagttagtttactcatatactggttcttattcacatctttgtcatgagtaaaaagtgttagaaaggccacgagtaaatatgcattttatttgtttatgaattcaaatactaaaagttttttatttgtttaattaagcattgacattgtctttttaaattcttttcattttaccttcttccctcttccttatccaactaaagacgcaaagcaggaggtgttaaaaaacaggtttaccatatcagcagtaacatagtttggacaacattacactttggttcaatgatagacatagaagtttgaacagaaatatgcaaagcaagtttgagctctaacttgaagagagcctctgggtgcctgccaggaaacctcacgagtggacccttaacattcatgtgtcaccacaaactaggggctgccctttagttttgaccagtctcagtgtcactcacttacccttaccttttcaaaaaaaagtcctaagaatataaagtaattcaatggttctacaattttagcatgtaactgagtcacctggcagggttgctttggtgagctcaagataaaattttatcagcatttctacattttctggaatattccttaatccaggcttttaatcccttggtgcttttctgaaccactgcaatgagcttctaactgttctcactgtgtgcaggctcttttccttctaatctaatttacacacttctgaacacaaatctctcacagcctgtttccttcatgttacctccagctcaagactttttgcctacaaaataaaattcaaacttgttagctaagcaccttctcatgtctatgctttggctcatatttcagccatcgtgtgccccacttattcttatagccaacctgaaaagccatcttttataagaaactacctctgctctccatgattggatataattaatcctccttccacatcacctcgccacaaaattgtatctgtgttgatctcatgccacatacctgtatgtattttatattataaatatttgcagacttgtttaatttgccatgttagactaagttccatgaagacagctccatatccattccatttttatatatccacaacatttggtcgggttgatgcttaataaatgtttattgaaggaacaggagtctcccacttctgacataatgaacttatttcccccagtgttaaccctacatctggttcctgtccaagagtctcttcccaaatcattctgattcaactgttcattctgatctcattaaacatttaaatgatatatctaacttcgcttgctttattctatgctcatcctgcagtctcctcataacttggtttcaatgatgcttgcttctagagaaaaaaatgtattaaataagcttatgattcagtcctccagctgtgatggttctcactgaacattagctcagtggttttcgaagtatggtctctagcataacctagaaacttgttagaaatgcaaattcttgggctcaccaagacatactaaatcaaaaattctgacattggggcctagaaatctgtgttttaacaagcctgccagtgcagcctggtcccttttcttctcggagccccactcaaagctttcagtgctcatctcccaccaatgacagggtcctctatggaaaccggcaggacggtttccaactctaactacgttttagagtttgcttcctagggctatccaggcaccaagtatcacaggttagtttcccagggaagcagactctgagacttgcatgcagggagtgtctctggggtgctctcaaccaacaccttcaggaagagaaggaagcagcattgggcagaggcatagtcaaactacagtgctgttggcacagaagactgaagggagtcagagccagggggtagaggtgggcccttagcatccatccttcaccattaggtgtgagttgccccacctccttgatggtgtaacctcagtcccaaggtgggtgggagtgcagcagagcagcccctacaagggccaaaccagagatacaccaggcgccagaagtgctgccagggaatagagaggaaaggatgggcttaaggtaggatccacagaacttggcaatggattagaagacaggatgagaagtgacaggttaacactaacacagaaatgtctaacttcggtagataatggtgccattggctagaagaggaaaccgaaatgaaagcaggttgttcagggagacaaaagttcactgtggacatctcagcagagtgattcagtggggaaaggaatggatgcccagaccacctcagaggaagatctaagctggagccagcaataaagatacaagatgaacaatccctaacgaactgctcctcagccatgctccccagacacgctgcttcagatttatagtccgggtgaggctaggaggtgcgcctccctcagtggaggacagcaaagcaccagtggctccagggagttaaaatcttttgataatttttgttctagcatctgtctgcagagctgtctctcagccattgcctgcctttacacaggagtgcagtccgaaattgggagatgagtgaaatttattatgcctagagatctggatccccagttgtttgggagtatattttctgaaccacttgttggtttaagtaatgcagatttattgatgccacttctcttgaatctgtgactctggacccaccatctaagtgaatgtgcagagggaacggaatggctgcaatagatctccattaaaaccagtgcatcctcccagacacatacagtagtagggaggtgagtcaatgtcaggacagcaccagctcccgcttcggtacatttccaaagttctcagtctgtgtacaaaggtttgctctggggcagcagaaatagccctgggcaggtagtcaaaggcctggtttgatttcctccacttccaggcaagtcactcgaaggctcacaggctttttcctcacctgccacatgggtccagtgagatctactgagctgtaaataatgaaatgagtgtgtgtgcagtcatctataagttgtaaagtactagaaaatggtgaaactttgggatttgggctatttaaggctgaatgctaaaaatgtcaggcattgtggagaaaggaatttaaatataagattgattgactgggatttaaagacaaatgaaggcacacacgcaagtgcacacccacactgacactgcacagctcccgttggaggcatatcctgaccatgcagacctggggctctgcctgtccaagtgcactcctttactacataaaccctccttctcttttggggctgtcaccccaccagagctggcaccgagcccttgctgctgcgcttccctggggtgtcagcttttgacagggtgtttcctccctctgcaggagccttaacatcccttggacttccttccccccacccacccccagcagttttatctcttcctaactcgggaccctttttttcccacacaaagtttattgtcagttgctggtttcatctgtttgagcggctgcaacaaaataccatagactgggtggcatatgcacgacaaaaatttatttctcacaggagaagtcaaagattaatgcaccagcagatctggtgtctgaggggccaccttctggtttgtagatgatgctttctagttaaaacacctatttaacacactattaaacactaagtgtgttaaatagtgcagttgatgtatttgtcatgtcacctttatcatacactaaatccttctttgtctttttttctgtactctaatctctttctgtaagtaatctttgcttgcagcagtaggatatttagagtactgtggcttgacaatatatttagtatttcaagatttccatgaaattcttctgatgtatgagttccctagttaatcttacatatgtatccctttgtaaaaacactttgaacatttaaaatgatacatgaatagtactctaatacaatgccataaaaattataaatcatttgtatagactggtaagtaaagattgtgagattaagaaacgcatcaaaggccattgagctggaaagtggtataatgagaattcaaaccagggtctcttgactcaaaatctaaggatcataccatttctcatgataatatgagtattattgttatctctatcccatagacaaagtgttaacactgaatgagcagtgaaatagtctcagaattttttattttatttagcaattcacttgtcatttctggtcctcagtttattcacgagtaaaataaaatagttggactagataatttctatagtacattcttacacaaaaaatctatgattttgttatttttaatgtgatatactcatggcactcattcacctcattttcccagcctgcctcactggtcattacttctctgtgttctttacaggctccccctcctctacactgccattaaatattgaaacacctcaaagctttacttatgtccacctctcctctgacactatcattctgtctagatgatcccatacatacatgcccattacttcaacctgtatttatacgccaatgattcactatatttccagcctagacattcttttgtactctagttaccagcttgatatccttacatggctgtttcaaaacaactcaaatatattatctctcaaaatcaaactcatgatgtccccacaccatcctagctttccaccaacaatacctatccctattaatagcaataccatttattcagttatccaaatcaaaaacctagaattcatccttaaaattctactatcattccaaatatcctatccatcagcagccactgtattcttaatcccctgtatttccttcaaatccattcacctctctccatatccattgctgcatgactatccaagccatcgcctctaccctagggtaccaaaatagcaacaaacctaatctgttcatttgcattattttttctccaaaactgattatctatatgtagcaagacagattgttctcaaattgcaaatcccactatattatcctcttgcttcaaacacttccatggtttcccattgtttatgataaaaccaaatgcttcaagttcgaagaccggcatgattgggaatttcctgtcaccctagcctacttgctctccatggtacagttgcactggctttctttcattccttaagtacaacctgtttcctcccacctcaggactgtgcatgtgccattcattctgctgaggagcctttttccttccacttcaatcagctaagtctgattcttcctgacaatctcagctcaataagcatttcctctaagaaatgtctctaatatcattaattggctcaggtccctctactgtattgctgcacttttcacagttataattttacttaattatgaatgattatttgattaggtctatttccatccattagacataagcttcatgatggccagattactgttttctatccatcgttgtattccaatacctgacagaaggagggcgggaggtggtggcacacaagagatgctcaaaaacaattgttgaataagtaaatgaatgaggccatttagaaataacgaaagtacctgtttacaaagtacatgtatcaaaactatgaatgcattctacttacatggttttctccaaataaaacaaaagacttcaatcaggattaatacctgggataaactgagtcattaaatctctcctttgccatcaggagtgacattgaaacaaatgtctgcaaacaacaaatacttttttcccaaaatatattgaatggcatttccataaacaaactagaacatgggaggagaaagaaagcaatattaatttaaaattaatcttatcacataacttataccatcagggatttcgggtaaaattcctttcaggcacatccatttaacaagaattgattgttactgaaagcctagaagagaatttggcacatacttggtgttcaaatatttgttgactgagtgaataaatgatgcaagtgtctaagaaacacaaaataaggacatgattacagtcacggtggagttcacagtcatctccaaaatgaggatatgcatcccagggaggaccaacaattcattggagtgctgaaataaaatactcaaaggtcattttacatgtattttttctctaaattacttttcttaagacacagaaaacaaaaaaagaaacttagctttgttactttctaacaaatagttaaatcattaaacaggattgacactagcatccttgtttggtcttatgccttaggggaacatgaaatgtgtgaagacattctgagatctgagggaagggtagacagtaatacagtgggactgaccaggcttcagcacacctttacctcctctcagcagatttcagtgatgagcagtttacaactagattgaaagattatattatctagttctaaaagaaaactaagcctcccaaaagcaacaagggaactgagaggaatcctgcaaaacaaaaacaaattttaaaacttgcactttgtaataaccctaatatgtaatcacagtaatgaacagtaagataatgacagaactgacatatttccttatctattaaagccatattaacaggtaaagcaatgccagtcagtggtacacttcttagaagatatttaatacatactagacacatacacacacacaacattttccttcaaggtgtatgtatcagaaaatcactttttaaggccggatgcagtggctcaggcctgtaatcccagcactttgggaggccgacgtgggcggatcatctgaggtcaggagttcaagaccagcctgcccaacatggcgaaaccccatctctacaaaaatacaaaaattagccagggatgatggtggatgcttgtagtcccagctactcaagaggcagaggcaggagaatcacttgaacctgggaggcagaggttgcagtgagccaagatcacccattgcactccagcctgggcaacagagtgagactctgtctcaaaaaaaaaaaaatcactttttagataaaattcatgctatagagagaagactatgaaaatatgtttagcaatgtgtccatcattaggtgattgagtttccttttgttttgttttactgaaaatcatataaagtatgttatctgtaaaagttctctgacatgcacacataaaaatttgggagaaaagattaactataatgtttaatagattttgtacacatttctttaaaaatatataaaacacaacacctttcaattggtttgcaagaataaccaattgacatcatggaaaatggaaattcacttgctgaattttaacaaaaatttgcatgatgagtgagactgacaacttagtgtcatgatttaatgaattatgccaatggtaaacttcatgcacatggggccaggtaattatgtggaaactttttcaatgcttaaagccaagtattgaaattaaacttagaatcagacctttgaaccattttatgacaatgttcaaaaattataaattctatccacttatattataatattaaaaatatcattacaaaaaaaacctgtgtttattttataactcagcctttttaatttctaatttcataaatatattataatggatattgttagtaatgtagtattattacatgtatataatttataagtaaatatacatgttttggctactcatgcataaaatgtttcacccataggagcacataatcagaaatgtctggagaccattatagtaatagatagatcatattgccacatattttatctcctccttgacaactgagctttccagatcttctggtgaaacgaaagagaaagttgtaacagaagagtgattaaaatgacaaaagcattacttctattacttctattctaataatatgagcaaagctataactatcaagtaataatgcactaaagaaggtgattaatctgatatattcacaggcaactaataagacctttctattgcagccatgaaaaatatgtgacaattatagatatcctgtgtgcagtgtttcaacctttatgtgacctgttctactaacagatttagtgatgttcactttgttagaattttcttacacatgccataacttgcttcagtcttttgattatgaatattatggatattaaggattctagactattctagatttaaaaaataatattgtcacctcaatcagaagggaaatattaaatagttctcattttttcaatgtttactcagtttttgtccaatgtaatgaaagtgtcagcagtacaggttacaaaataaaatgtgtattaaagtaaactcatttgaacaggttaataattgtagagggagggaaaaggctaaaagattgaatgtaaaacttatgaaaagtagatacatcgtctctatgatttgcagtagtcaactgcatacagatgaatcattttaatacacgttaactactttccttttacagatggagaaactgagaggaagaaagtttatatggttcattaaactttgtgatgcaagctaaactaacctgtctctgtattttccatctactgcccttatcactatctcattagaatactcttcaagcatctccttactgattttcttaccaagcatttgttaagttctaatgagagttggtagtaacattttcacccactctgtgaaatatgaaatcttattcataggcctcttcttttattcttgtatttgcatatcaaccaattaatcaacttgctttctttatgttgcttattatcttagtccttactaaattgcctcttaatgttgtccacataacagaaatgttaaggtggatacttaacattttagtccagtctagccggtgccagtgcaatgccaaatcatgaattaaaatataattacaagaaccacttatcaaattttaacaattccttcagctttgtgacagttttttctacttcgattaaagtcaagtaaaattaaagttaaatatttttattaaaatatctcctttaacattccatattaataaacatattaaagctcatgcttctaagtagattactagaagttactttatcgaattacagcaatggttaattctagatcatagaatttagaatgactttttgccttcttcttttttttccttttttttaaacagagtcttgctctgttgtccaggctggagtgtactggcgcgatcttgactcactgcgacctctgccctgcaggttcaagtgattctcctgccccagcctcttaagtagttgggattacaggtgcctgccaccacacctggctaattttttttttgtatttttaggagagacagggtttcaccatgttggccagactggtctcgaactcctgacctcaagtgatccacttgcctcagcctcccaaagtgctgggattacaggtgtgagccactgtgcctggcctgactttttgctttcttcttaatacttactagtatttcttgaatttttaaaaaagaaacataaagtactttgataaaaccaacagtctcattgttcttaaaattgttcaaaggttctctggaaaaaaaaaagaaaattatcatttggttaagaatcatgttggtctgacatcaatcatcctataggagtgaatattgaaaaagtaagatatattgtggtataatcgagattgcataaattttaccatttttgagaagaatctgctccaaatcctggcttaatgtaatatccagcatgctacttaattttcttgtcttcaccttttcatatccacatccacctaggtgccacctcacagtataagccagcataatccattcttctcaatgaaaccacaatacatctgaccctgcatctcaggagaactgtatcagccacagcacttccagttgactatgaatctgaatgttatgcctcaggagaaacatccttgctgggactgagtagtgattcaaggagatagttatgattcagtcaagaaattaataattagtgttatttttattattgagacagagtctcgttctgtagcccaggctggagtacagtggcatgatctcggctcactgcaacctctacctccccggttcaagtgattctcctgcctcagcctcccaaataactgggacagcaggcacttgccaccacgcctagctaattttttgtatttttagtagagacggagtttcaccgtgttagccaggatggtctcgatctcctgacctcaaggtccacctgcctcagcctcccaaagtgctgggattacaggcgtgagccactgcgcccggccataaattattaactgagccaggcacagtggtacacacttatagtcccagatactcaggagactgaggttggagtatccttttttatgttattttatttttaattattatgggtacataataggtgtacatacccatggagtacaagtcatgttctgatacagacacataatgtttaataatcacatcagggtaattgggatatccatcacctcaagcatttatctttctttgtgttaggaacattccacctccactcttggaataggcaccctgttgtgctattaaatacgaggtcttattcatttcatctaactatatttttctacccattaaccatcacctcttttcccctcttccccactacctttcctgtgaggctgcaggattcttaagcacaacagttagaggccagcctggacaacatagtgagactcaatttctaaaaaataaaaaagaaattaccaactaatgctaaaaaaatagtctctgatgcttaggtatgaattagaaatgaccaaaaaaaaaaaaaaaaaaaagactgccctttgcttccttctccccttctcttcaagttttccattgctactcattttagtctggtttaatcaggtttcatccattaaaagcaattgttgggatcacacattttgagttgtgtcagtggacttccctcatgctggcatgattcctgccccaagcccttagtaaaagccaccaagccatataacataatctctcattgagtaaaacatctgatgtgtttagaatgacttctagcaaaaaaccagcctgtccagcatcatctctgtataacagataaaggaataggtactgcatcaaaaggttatagaacctgcccaaatcaatcccatgtgttttgcaatggaattaggttgaactaaagtgaaaattcagttttctactcctcattaacatgtctcatgttgcaaggttgagaggaaggagaagaagaactgtatttacagagagattccccctctctttctttctacagattactaaaacattcaaagaatcaaatttaagaaatcagttcatcagagctcatgttgccaaactgaggtgagtggaactgtagaaaaaatatttaagtatagatacaatgtggcatacttgactttttgtcacagaatgaatagtaaatgacatgttcagataagttgttgtaatattatgaaaatagtattttagtcagcttaaaaaccaatgccaaaaaagccaaacatatgatctatttagctactaatgtaaataaccatattatatctattcttattgggaagaggaagaaggggtggagagagagttggggtgaaggtacagtaacaaggccatcctattgtaaaactccagtggatatcattcacagtgcagcctatgtaaacagtccctcctggagttgtacaatgctgtggtttgggtgtatccatccaagatcaagacactatgaccaacatcaaaagtggctttttggttttatctgcctgatgtgctataataaaagggtattatggccaaatccaaggcatgtctatcatgaattaataataggaggagtagcagcatgcatgctagttatttgccattcctgccttagttaaatatgatgtgataaaaccagcctttccaactgaaatagtcacctttactgactctcccgcaaatgtctcaaatgaccacattgctctagtctttaaataatatgcaatagttctttggtagaagaggaattatactaattctttctcaaatactagcatcacaagaaaattaattcttgttctctggagagtcacctagtaagtatctggagcacagatgtctggtcaggtaagttttgatgaggagttaaagggataagaagagtccatgagaagggtattttccaaaacacctttcggtcaattcagtgcacattcacttagtactttcttgtcagtatctgtatcagccactaatgttcaaaagtgagtaagccctgaaaacctgtaggactacatgagccttctgccttttctctccttttgttcacttcccacttatcactcaatcctctgcaacctggcttcaataccaccataaaatatcaactgctcttgccgattcaacaatgacatccagataacaaaatccaaagaaaccacatcagtcctattcttggacctttcaacagtatttggtcctgttggcctgtcactccttgaaataggactatcccttggtttgcatggccttgtataccctgattttccccttacctccctagctattccttcttagtttcctttactaggtcttacttctttgtatattccttaaatgttgctgaacatcaggctgtgctctaggcctctcatcttctcaggtcacactctctcctttccttggccttcactgccacccatatgctgagtgctctcaaagttgtatctctaggccagtcctcttttgcctccaaacatgaatatatgcagccatctacttggtaccatcacatggataattctcatgatctcttccagtatgactgcttctttatttttttctgggctcttttttagcattgctttacatggaactttatcatgtctctcaacctctattttatcttttatctatgtatgtagagtctgtgtaatttcttcatctcttttagataactaatatctcttcagctttgacttgtattctgtgtaacccatttattgcgttttcaatttcaatgagtatgttttcctatctgcaagttctatttgtttcttttgagaatcttcctggtcttttaaacacatttcttattttaatttttgggggtacctagtagttgtatgtatttttggagtacatgagatgttttgatacaagcaaacaatgcataataatcacattgtgtaaaatggggtatccatcccctcaagcatttatcctttgtgttacaaacaatccaattatattcttttagttatttttaaatgtacaattaaattattattgaccatagtgactctgttgtgctatcagatactaggtgatcttttaaaaataatgttttctacttaatctcatttttatgattccctcttttacgtcatttgtcatttcaaatacagtcacttgtctgttgattctattatgtgaagtttttgaggataatctttttgttactttgattccaccttggtatggtttggctgtgcccccactaaaatctcatcttgaactctggttcccataatacccacatgttgtgggagggaccttgtgggaggtgattagattatagggacgtttcccccctttgctctgttctttttcctgccaccatgtaagaaagatgtgtttgcttccccttctgccatgattgtaaatttcctgaggcctccgcagccatgcaggacctcttttctttgtaaattacccagtctccggcggttctttatagctccgtgagaaaaaactaatacacacctcatgatgtattgtttaccactgaaattgtatgcttaaatttaatctcacttgggaccctgtacaacctagacttaacatatctacctccagagcagttacatctgtcagacattctagaggaatcagcagcacatggactttgttgttgttaatttgttgtcgggggaggggggagggatagcattaggagatacacctaatgctaaatgacgagttaatgggtgcagcacaccaacatggcacatgtatacatatgtaacaaacctgcacgttgtgcacatataccctaaaacttaaagtataataataataaaattaaaaaaaaaaaggttctgggagtattcaggtagtattaatgaagattcagacatcgtgcagccaggcccatgcttatgaattttcaggtgatacttctttttcttttttcttaatttaaagctggatctcggaaacagataaatttatttttttatgacatgacgagcatttttttcattctagttcatgctgttattgggtgtttagttctttgagactcctggcctttttctaaaacctcaagttcaacttcctattttgcactggcccaaggtcccatctccagtctctatgtaaatgctaaacataagcctgtggaatattctagtctcaccacatactattcacattcttctttgtttttggtcttccaggattttccttacttttctatgaacccagtcttgcatttgaaatggaatttattatatattatctatcctttctatttgttttatgcagaaagtgttttctaaaattatttaggcttccatattgctagacatggaagttgtaattatttgttcagtgcctgtttctacatctaaactgcaagacccatatggcaactgtgaatcttagtcccagctaatttctgaagcttagaatagtgcctagcacaagaagttgtttatctaacatttttaaaaataaatattaaattcatatctggaatgaatattaagttagagctggtcattgaggtgagaggaggaagccaagagagaatatgagagcctcaaagccaaatatctttaatgtactttttcagaaaagaagacagccaatgtcaggtggaggaactggtttatgaggtaactttcctggaagaaaatagaaattactgaggttttagataatccaaatatttaatcaagtcaccaaggtttattgtggggaatctttattattaattaaaatgagtgatgaaatcttaatatacgacaaaagttaaaatttgcttttgcaggcagatgaatggtctaggtatcaaaaaattaagttgagtctctaactcacacaaatttacaaccctatcactttatgaatttgtttaggagattatttttaataacactggtgaagtctaagaatagctaaaatttatagtacacttattgtgtgctattgactcttctttgaagttttgcatatagtgattcatctaatcttcataacccattttacatgtgaagaaacttagatatagaaagattaagaaacttacataacttatccaaagttacacagtaaaactctggcattataacttcaaaatcagctatcctacagtgagtacagtgttctgtgcattgaaatcaaataagtgagatagcatcgtgatatagtattacgtatgcaaacactgttacagagatctgtctaaagttaaattccacaaatgaattctttaaaagggtttaatcaagaagaatatataaacaggatggtgaaaaattgtcatattatttgttttttaaaatatctttatgatttacaggcaagatggtagtggtgtgagagcggatgttgtcatgaaatttcaattcactagaaataacaatggagcatcaatgaaaagcagaattgagtctgttttacgacaaatgctgaataactctggaaacctggaaataaacccttcaactgagataacatgtaagtataatttttcataaacaattttatttcaatatatccctcaagtttaccaattcaaattcatattttaattgagaggctgacttttctttctttgaaactaaactgtgaaaacaatccattaaaaagctaaatataccatatagctccctaacgtaaatcattctaagacttaaagaatcatttggcatttatatagtaaattttatttgctaaaaattctcattaattatccctgcaacattccttatgagtgatgttactgtcagatgtcattagtggataggccataggaggggtacatagatgctcaaggtcagagaactatttaattaatgatccacctcagaggcttcttcatttttctttgtaacatttatcacaattgaaattacaaagttatctgtgtaaattttgtattgtttggcttcatcctacactgtaatcatcctaaaagaaagaaccagtcaaccttcttcatcctactaccctcctaccacccagtctccatcatataacacatattcaataaataattcttgcatgactgaaagaaaagaaataatatatgcatagaatttaaggacattcctccaagttggttacattctgctagtttaataagccattatttcttctcgatgagctcaagattaaaaggattttgatgattcccatactagactggtaggtaccagttacagatgtactaactgttaaatattgaaatgctttcctatttgttggtaaacaattactgcatcaggcccacaaagttgtcttccgagatgtttcaaatccactgcccctgctgctaaagagttatgcttagcaaagcaaagcactctaagacactgctccaactccatggcctgattgcatcttttatgactggccaatgctcacgcactgcagtttgttaggtagttgaatattacctctgcttccacacattaaggaatgctcccgaacgcacttcccaagtgtttatttatttatcattatactagacaatatggtgatacgatggtcacagaatagcggtttccacctccagagcccataatctagttgaagggaaagatattccaacacaagagtgttgacaatcaagatagaatatgatcaagggcccagtgtgaggcccaggcaatgatcactgcaggaatctggggaagaaagagaccagcgtgcttgggatatctagcaaaagtttcatgaaggagaatggactttgactttgaaatatgggtaggatttacatattttgagatgagaaaaagaaagttcccagagaaggaaagcatgaaaaggcaaacagtctgtactgaacgcgatgctttgacagaataatgaagaaagggacctgctggaatgattgatcagtgttcatcattcacaccatcatcatcaaaacacttatttaatgagaacttactgttttttaggcatggctttaatgccctatatgaatttttttcttgattaatccttacaacaaacatatcccatagatagttttattgtcccccttagaaaagataaattgcctaggctgacacagtcagtatatgaggcagtcaggattcaaactaagtctgtttgttcaaaaaattaagaatggccagctttttaaaattttctgtctccagaagtatgatttggctccactgaagtttgcaaaacaaatgtgatacccaaaccttgtgaaacttttagtgggaaataactttgcataagtcggtttgagagagcgtggaaacctgtcttgaaaagttttaatttaacttgcaggaaataaaaatgatgggtttctcaattaaaaatttcaatcaaggaaggatatgagctaacataacatttttttaaaaagatcagtctggtaaggtagaggtgcataaactgaaaaggagcaaaagtggtggaattcagttagaaaattattgtaactgtactgatgtcaaatgatgaaaccatgaactaaagtagtaccaaaaggagtgaggaggatggaataattcaaaagatagaggacagatgtgcagaacctggagattataagatgtgaaaggaggagtttgagaaaatttcagattttggaagtggtgtcattttactaaaaggatataataagtagcaaattttggataaagttgggtcccactgagtttgagatggctgttggacatgcagagaaaactgtcttgtatgctgttcttaaattgaaatagacagacctttaccctctgatactgacatattttcctttccaggctcaccctccatttccctaaacacaacacatgcactagctctccttactttattgctccacaaacatcttacacctccaagcatttgtgcccactgtaccttctatctggaatctcttttgtcctcttgtgtgcctgaaaaattcctttcagatcttcaaaatacagtgcagatgctatttcttctagctcaaatattatctcctccatataatttaattactctcttttttcttttctctactttgcacttacatttatttgaatgattgcttgattaatttctacctgtaaattatgtgagggcaggtcctctatattttgctcgcagttaaatctgcagcacttattatagagtggtatcattagagtaatatacatatatttgaggacatgataaattaacttcccctatagtatttatcacattgcatctcaatgacttgcttatgtttctgttttcccatataaattgagtaacttgaaaaaagagatatctattaagtatttaatgagaaattaaagtacaaactttagtatgcataacaacaaattgggaaaaggttgtaaacaaagagatttgtagggcccatgagttagagatcgtttcagcaggtctgaaaggaagcctaggaatctgcattttagaggaccacctcccaaccccaacaagtaattctgcttcttgttgtctgggtactgtactttaagaaattatggtgaaatgatatcagcctttattgtatttatcttattctcattttttaatactagcacttactgaccaggctgcagcaaattggcttattaatggtaagttttaatattattttgtaactgtaatttgccaaatcataaagagtaaaagtgcaagtcttttgtgtacttttggccaaggcagtatctatcaagttgatgtctttgttcttagttcgctcaggtggtgttgaaacaagacagtgctgatcccaagtgtcccatggagtggactttaggtttcccctttccttttagaaaaaggaagaagttgtagtggaggactacccactctgcactcaaaattgccctcatgaaaatttctttggcagctttgagaaccttttactgccctggttctaaggtggcatttctgtagacttacaaattatgtttgatgacaccgtttatgtagcttctcctaaccaccagagtagcttgctttgttgtgaattcaggttaatcacaaagtataataaaaaagaattgtcagaagtcttcccagctttgggtctataacctgaaggaaaagtcactactcttcaacatcatcctatgtactctcaggctaggatagcagaaatgcaatccctagaaaacagcaacttacttctctgaccaaaaaaatgcagttaaaaattagttcaatgtacctggtagctggcctatcttaggtacttcagtgattttacaaagtgatggtagtcctatgggtgtttttcagcttcactacgtatttaattcatgcttattgttaatgaaactgtgataagcaatttactagggtatttgtttgggagatgccacaaaggaacacatgtatctcttaatggaagcctggtcctcctttatccaggaaatttgctaggaaaaaaaagcctttaggtggttgtgctattaaaccagggcactacttaaaagccagcccagcaatagttgtgtgatttaccattaatttcttagtaatagaccacacaaaagaagaaaattatgggaatgcgagttgagaggaattgggtgatcagcctaccccagcccgtttcagctctggccagtagactattcacgagctctttgaaaacatttaaataaaccttatttagatactagaaaccctctgtcaccctcaagaatattctgtggtatagcgactcctttatgagggcatgtttggtaatacagcatcagtcttggaggtggactggattctacaaggtgaactgcagtcactaaggagtcttttggatgagaccagttttcctccaacttcaatgtgtgcatgaacctcacatcaaaatgtagctttagatttgtcccatgatgtggttccaagaatcagcacttctaataagtttccaggggatgcccatgctgcaggcccacaaaccacactgagcatagcaagactattgagaaaaaggaaatttcccaggagtctgtggcctgagctggcacatccaataatgacctatcttaacctcaactcatgaggaattccagggaactctgaagctgctcaaaatttgaagcctatatgccaactaaattcagaaatgttctccaaaatgctatctataagcaacagtagtcacaaatgcattgtagaaatatatcgatcatgctttttggaaaatccagcatgtcctgaggaagaatgtataagacataaaagtcataaattatggaaagactcttcagcttcttccaaatgtaaaggaatcatgatcttcccagcacattaatgccctttctcattagaatgtggggccggtccagacctaataacattgtctgagcagagaatccttggaggcactgaggctgaggagggaagctggccgtggcaagtcagtctgcggctcaataatgcccaccactgtggaggcagcctgatcaataacatgtggatcctgacagcagctcactgcttcagaaggtgaggccaccactacctacccatctgggaacaattagaatagacaggtcatgaagactgcaccctctaccctaggattgaattgagccagaaataattcaatgcaaaaaaatcagtaagaattttcttcctattcatgaaaggaaaaggatttttcccctttagcatgctaatttagtgctatttctctgtttcaggtaataatatattagcacagtaaagaacaaagatttatatgtcagaatgttttttaaatcctagctataaaagcttaagaaatttactaaatctccataagctttattttttttccaaattaagggacaacactgttatctgtgacttagtgttactggtagcattgagtacactaatgtaaacatacgttaaatgttagcgaaacgaattgctgtggaagatttgcacattatatcatgggagctgatggctaacctagagactgccccatgccattaatttattcattcataaagattattgagtatctagtatgagcacagtgttatatattgtagaagctactagtataaacaaagtattgcctctgccttcaaagagcttacactcgaatgttggaatcagaatgcacaaaaataatgatcaattacaatgagtagcataaataaaattaatgtaggcaacttacaagaattcttaattgaggtgactaaactattgccaacactagggtgatatgctaccagtggcgagtaggttgcataaacttaccttattggtaaaaagaaaagttcacattgctcataaaagaaggattttagatttcagcataactaaaatctgtttcaaacctgccttgttactggggcatcgcagaccacaacagttgttgggaacttaactcaaaaagttcacccagaaaaataatggagatttgaactcgtgtgcccctgaccatatcaattttcttctcagactcttactctaaactggacctccttatcacacacacaaagccttccataggcagatcaatccagtcttatttctcaaagcatgtaccttgagcttcagataaacagcattgttctcttcccctggactcttcctacatttccctacctatgagtatctgatcaatctgcttatccttgaaatgttaatatatttaccacatctctatttgaattttatgaaatttttgataatttctaagtagttttttcagatttataggcactacttcatggtacagtgactgttacaaacgtatttgttaaatttagaaggaataaagatttaaaagactagggtagttactgaactaaagttttaggaaatcccaaattatttcaaatttttcttatggtaattttatgacttaatatttttatatgcagtgaacaaatttgaaactttaaaagatactcccagaattatcagttttctgatgtagattggcaaatttattactatatcccaaataacccaagagacaaaattcacaaaaacatttcaattttcattgccacttgaaaggccaaaaagcagaaatggcacgcattgatttcaatcgtactcttgagtgtgggaaccaggaattaaaatacctggacttatcaggcacttagcataaccaagaacggaatagaaacctccctggattctaagccctattcagtcccaatcaccaaaaaccaagtaaacgatatcactataatgaaagccacagttataaatatcgacaacgattaccaaaggaatccatggaactttgaattttgccaccccacatccttctattcattaccatgattgatccactaaagctaacagactctgtgaaccttgtattggacccctccctaaagacctgattgtcactgagaaccatcagtgaggatttgtttggggcatgaccagccttacatcaaagtacatagaagtgatgaggtcttatcaaagaggattattgaattatcacctcttctatgtagctttccctgatactctctttcctctccattgagttccacagaaatttttttatctgcctttaacagttgtcctcatgatttgtgatatttgacttacctcttgtcagtttccttcactagtgtagagttcctcaaagaaagagaccataattacttatatttttattcctggagactcatactattccttatacaaagtagacacttaacaatggcttgttgaactataattaatgaaaataatagctaccttcatgaaagttcactttgtgccaaacactatagttgacataatacatttgtctcattaatacttaacaattgtgtgagaaggtatcaccaatcacattttatatgtaaataaaccccagagctattaattaacttgtcataaataacacttttcatatgtggcatagccaagatttaaatataaatgttactggttccaaaatgatgctctaattcacttgctggaaagaaggaaaggaagaaaataaacgagtggaaggaagagagggagggaagagagaaaaggaaggaaagaaaaaagagtctcttcagaaccttcactgtaaagactccgagcaaaagaagttgaatataaaaacaacataggtttgtttgttttctaatattttttcttcaaaatttttaactcaggttcactcttacacaaactactgtgtcttataaaagtatttccggtcatagaatttttattttctgtattaactccactatctaatctccataaaactcctaaattggtattatcggtaacattttgtttttactcaacccttaggaacaatgttaagttaatcagccctccacatcacagatccttattttcatcagtctgtacaaggcatttctctcattttaattttttttcctcctgtcatccctggatttcactttcactgccctccttccacccatatgcctcatactaatatattcgaaatatacatgtcttaaaggtacatgcacgcacctacaaaacctatagtgtttttttgtatgtatatgtctttaatttaaataagtagcattgtgtaaaagtctaatattgtttcttactgttttcactcaattcttggaattttcatctgatgcactgctgcatagcaccccatggtatgcagccaccatatttccttcatccaattaggttgcatgacctaccttcccattgccacaaagagtacacacaaaatatttgtacttatctttctgtaaaccttcaggaatttcagaagcacacatgcaggctgctaaatataccagaatactttccagccacttaaatctttaccagtattgcaaaagaggccccatttccctccacatcaacatttagtattattcttttgtttaagttttatcaatcttttaaatgtacacaagatgctcatttttataattttaatttctcagattactagtttgagtatcttttcatatatctaagagctgttttgatctcccctaccatgaactgccactaatattctttgcctattttacaatggtttttctgcttatttattactggtttacagacttttaaaatatattctacaaaaattttagacattaaacattaccaatattttcccatggttcctcatccatctggtaaacttgtctatggtatatctaattttgatttaatagaattcattctatttttaccttttagtttgtgtttttgttgtttagccaaaaagtccccattcctaggtcataaaggtaatgtcctttttttttttttaacgctactgttctctctctgtctccccctatgtatataggtgcacatatacttgtacacacatacatatacctatatatgaggggagttcgataagtttatggaaaataaaattaaaagataaaataaaaaattataaactttatttctcaacataagctccttcaagttcaagacacttttgtaagcaataataccagccatatcgtccatccctaaagaactgagggtcctgagaatttaactatgtcaatgcagtcttttttacattacttttttacagtacttattgatgaaaaatgggtgccttttaaagattgttttaagattagggaacaaaaataagtcagaggaagtcaaatcaggactgaaaggtggatgcctagtgatttattgctgaaactttcataaaactaaccttatttgatgagaggaatgagcatgagcatggttgtgatggagaagaactctggtggagctttcctggacactttttctactaaagctttggctaactttcttactctcataagaagaagatgttatttttcactgaccctttagaaggtcaacaagcaaaatgccttcagcatcccaaatgtctgttgtcatgacttttgttcttgactagtctggttttgctttgactggaccacttctacctctttatagccattgctttgatggtgctttgtcttcaagattgtattagtaaagccatatttcatcttctgttacaattcttcaaagaaatacttcagaatcttgatctgacatgtttaaaatttctattggaagctctgaccttgggtgcagctgatctgggcgaaacagttttggcatccatcaagtagaaagtttgctcaactttagtttttcagtcagaattgtataagctgaaccagttgagatgtctatggtgttgtctattgtttctcacagttaattgttggtcctctttgagacatgaacaagatgaaatttttcctagcaaactgatgtggatgatctgttgctgcgggcttcaccctcaacaacatctctttctttcttgaaacaaattatccattagtaaactgatgattgggggagatgctgtccccataaactttttgtaaggcataaataatttcaccattcttccagtttcaccataaatttgacgtttttttgcttcaattttagcagcattcatgttgctttgataagagctcttttcaaattcatgtcttattcctcttagtgcctcaaactagatcttgttcagtatgacaagttagtatgagtttatctgcatgcaaaaatctttgaaatccatgcatagtttgtttataatatacattttcaatgaacttttgaagaccccatacatacatatgtatatatatgcacacacacacacacacacacaccaaaatcttcaaccattatcagacttagtgcagaaaaattattcatccattaacaagataagaatgccccttatcatcactactatttaaatggagctcctggctaaaggaaaagacagggattgaaaaaaattagttaaatctaaaatgtttattatttcaggtttcttagttgcttaaatgggaagggaggtatggacaaaagagaaatcaaagatatttgtgttatgctacttatcattaaagtatcagaataacttcattggaatagaaaaacaccaagatcaccccacgatatgttttctaaaatcttctccatttctttagacaagtgaccatgtattcggccagtgaagaattaaactcacttgccagcttataatgcaggaaaatatagcaaagagatgtggatccaatagtttctagatagtggtacaggatggctaagatgaatttatatatctgaaatgttcacaaattccctactcatatagcatgttttcataatgttttagcaactctaatcctcgtgactggattgccacgtctggtatttccacaacatttcctaaactaagaatgagagtaagaaatattttaattcataacaattataaatctgcaactcatgaaaatgacattgcacttgtgagacttgagaacagtgtcacctttaccaaagatatccatagtgtgtgtctcccagctgctacccagaatattccacctggctctactgcttatgtaacaggatggggcgctcaagaatatgctggtaagtgtctcggaaaaaaaaattaacaatagaaatgtcttatatttgctattaggtaattttttaaattaggaaacatctggaataggtgtttctattcttctacagacagaaccattctatattctgctcagcccaagctctggctacccctgagtctccttagcaaagcaaagcaatgctccagaaactatgggaattctcaaatatagtaataggaaaatgtaaaagaaagttatgaagacacgagttctttaataatccagagattctataagattcaaatagcttccctataaacaataaaaaagattttgtttgtttgtttgtttgcttgttttttagagacaaagactttctcagactggagtgcagtggtgcaatcatggcttactgcagcctcaaactctggtcttaagaaatcctcttgcttcagcctcccaagtagctagaattataaataagtgtgtaccaccatacccagctttttttttttttttctacagacaggttcttgctctgttgcccaggctggtctggaattcctgccctcaagccatcctcctgccttgttggcctcccaaagcaatgggaggatttagattagacattgtatgagggcttaataatccttaaggtattaactgccctttaaagtattctgggatatggcaaaaactcgatgtgtatataaacattggtcatatttgtttattgaatgaataaaatggaaactaaaatgaggacaatgcacaagagctactagaaccagtaagagtatcagcgaaggagtggaagggtagcattgacaatttccctgggcttttacccatgttgtagattgtctctccaaggaataatacaaagccttaatagtcctagaacacattctattgtgttcttatggcccaaagtaaattggtgtagtagataacatttgcaccagtcatgaaaaactattggtgtcattctgagagtacatcaatataaaatagactagttctttagccttgaaactagactggtttctcttttgctgctaggttaaaggttattcaatatgtaatcttccaatccaaaatctgtcagtggataatttaaaagcttttagtcaattttaagatatttgttttcttaaaattttaaggggcactgtgtcacaaagctaaagaaaaaaaagaaaaaaaaactgatctgtgaaaggggttatcctcatctacttggggaattttggctgcgaagaaactccaaagtaaatctttagaagccttcattgttaaatatgaaataatgtttggagtacatttatttcttctcaaatttattatagggtcaataatgtacacatcttgaagtccatttttttcctgcttttataacaaacaggccacacagttccagagctaaggcaaggacaggtcagaataataagtaatgatgtatgtaatgcaccacatagttataatggagccatcttgtctggaatgctgtgtgctggagtacctcaaggtggagtggacgcatgtcaggtaagctcaagacaatctcatccatgtcatcatccaagaagtgtataagcacttcctagtatgtgataatgtgatagacataagtgtaacagttacaatacacagccctgttcctctaaaatttataatctagattttagaaataaatttttttatgaatgaagtttatctatcatgaaagcattaactctgagaggccaaattacagagtagttaaccatccaaagctcaagaatcagaaagacctcgatttgaattccttaacctctattaccaagtctctaactaaaagctggggataatcataatagcacctaactttttgggtactaagaaaagttaaatgaagactaaatatatcaggcacatggtaaacaacaaagaaatctcatctatttcactattattaatgtagaccatggtcactcgtgttaataactttaacctcaaccttttaactgctgtgaaggattaaataaaaaattaatcactatattataaaaattaattgatatataataaatgaattttaagagatacgtaataattcatggactccttgaagatagaaaatttatacaaaatcctagtaatttgagtcacaaaagctcctacaataatgaaacagtatgaatgaaaaagaaaagaaataactattatatttggatctagcccataatttttaaccaaatgcacaaaaacaaacaacaaatatgaaattctcactgtaaagtgattaaaatcaaatttgaattctaaaattttaaattaaattatctaaacataattgatgcagttatatgttttaataggttttgttcacatatctgaaatccaactccacacagtagcaggaacagctggtgtcagaaattaaatattcttttagtctggagttttaaaaaatcaatctgtttacttgagtaatttgttgctgttttcatgggtgaattgtatacagaaggataagaattattcttcgcatcaaaaggtcactgactttcatatttagtgctcatggtctttaaaaagtggataaaaagtagttctcacatttcatggaaagcccccaatccatgagcacatttcccaaaatgaaacatttttatcaactgcaagttgtgtgtaggtggagatttgtttttcaattgtcaagatactgttaattacccagtcctttatctccttttggtggagatgtctctgtgctaggaaacccttcttgctctccttcctgtttctcttttactactggccctgaaacaacaaattctcaagtttcatgacagctttccaaagaatccatcaatcaaataagcaacacaactcgacactgacaattccagacctactaagagcattaattaagacttaaaaataaacatgagttttaaaagggtgttattcattattttcccatttataacgtcccttaccttctgtccttcagtgcatacaaattattatcttccttgaagcccagttcaagccgtacctcaccatgataccttccatgtatattccactctaggcctcactgatttttaactgaaatactataatgcatagttcacacttaaaaaaaaaaaaaaaacacagcactttacataagagcttacaggatcctatttgttttatccattcttttgttcatttttacaatcattaattcaaaggaattatattaattactttctatgcacccgacgttgtgttaacacaacaatactatccctgcattcagcaagtctatggtctacaagagaggacacaaattcaaatgtctgtagtcaagcagtgaagctggctagatatggaaaaattacaagtccctcttgctttaacatttgcttgcccacatttggtcagacatcatgcaaaataatttctcactatagaaaaaaaaacactacaaaaacaataatataaagaactgagaactggttaactgaagcatgcatatgtcatctaaaagaagcaggtgacgaccagcttcatgaagtacttgccatgcatattggcacttcacacactgacccttctccccacctagaccagtaattaaacaggtatggatgagctagctactaagagcagccaactgaatagctgactaacttagaagcacacttggtaataatagctgacttttattagtactgactatactatatgctaagctgtactcaaagtgctttgagttttaaactgatacaaacattatatgaggaaacagaggtacagagagctattcaccagcttaccaaaggtcacatagctggtaagtggaggacttaaacccagactatctagtttcagaacccacagacttaatccatcgtgcagaacataagacatactccatctgtctccccaactaggttattatgtgcacaaatatttattggttggttggttcattattatgactgggtggtaagtatgtcattaggagtgttttgcttatgactatataaatttcttcaccaaaagaagactttctgatgatatactatgcatcagacaccacgcagggtgctaaggttaggaagataagtgagacttctagaaactcattcattcaacaaatatctcctaagggctagaagcttaggtttcagcagtgaacagaataggtatgttctctttcgtgttggaccttatagtatatctgggaaaacagacattgaataaatatcacaaatgcaagtgagtgtttcagagacatgcagctgctacatcaaaacaaaacagaacaaaacaaacaaacaaaaactgaccagtgggattaagtgtaaataggcacacaaatgcacaaatatgcttttataaaatagtgaagcagtgacagagacacacacaagatataaagacacaatgaagaacaattgagcccaaagctggaaagggtgagagtgtgaaggaaaaaggttgatcagagaagttttcccgaaggagagaaagcctggatgattaggaggcaaccactcggtgactgagggaaatctgaaaaatgtatttgtcatcttctcagacttgctgaaggaatgacttgggtactttgaggatttcagtaatttttccatgacttggtataatatttcaaaaggaaataggctgactttatttgtataatgaatgtgactccttcctcgactgccatagaaataaactccttaatattttgggtttgtctttgcacttaagtaatcagtcattctgtttttttacagggtgactctggtggcccactagtacaagaagactcacggcggctttggtttattgtggggatagtaagctggggagatcagtgtggcctgccggataagccaggagtgtatactcgagtgacagcctaccttgactggattaggcaacaaactgggatctagtgcaacaagtgcatccctgttgcaaagtctgtatgcaggtgtgcctgtcttaaattccaaagctttacatttcaactgaaaaagaaactagaaatgtcctaatttaacatcttgttacataaatatggtttaacaaacactgtttaacctttctttattattaaaggttttctattttctccagagaactatatgaatgttgcatagtactgtggctgtgtaacagaagaaacacactaaactaattacaaagttaacaatttcattacagttgtgctaaatgcccgtagtgagaagaacaggaaccttgagcatgtatagtagaggaacctgcacaggtctgatgggtcagaggggtcttctctgggtttcactgaggatgagaagtaagcaaactgtggaaacatgcaaaggaaaaagtgatagaataatattcaagacaaaaagaacagtatgaggcaagagaaataatatgtatttaaaatttttggttactcaatatcttatacttagtatgagtcctaaaattaaaaatgtgaaactgttgtactatacgtataacctaaccttaattattctgtaagaacatgcttccataggaaatagtggataattttcagctatttaaggcaaaagctaaaatagttcactcctcaactgagacccaaagaattatagatatttttcatgatgacccatgaaaaatatcactcatctacataaaggagagactatatctattttatagagaagctaagaaatatacctacacaaacttgtcaggtgctttacaactacatagtactttttaacaacaaaataataattttaagaatgaaaaatttaatcatcgggaagaacgtcccactacagacttcctatcactggcagttatatttttgagcgtaaaagggtcgtcaaacgctaaatctaagtaacgaattgaaagtttaaagagggggaagagttggtttgcaaaggaaaagtttaaatagcttaatatcaatagaatgatcctgaagacagaaaaaactttgtcactcttcctctctcattttctttctctctctctccccttctcatacacatgcctcccccaccaaagaatataatgtaaattaaatccactaaaatgtaatggcatgaaaatctctgtagtctgaatcactaatattcctgagtttttatgagctcctagtacagctaaagtttgcctatgcatgatcatctatgcgtcagagcttcctccttctacaagctaactccctgcatctgggcatcaggactgctccatacatttgctgaaaacttcttgtatttcctgatgtaaaattgtgcaaacacctacaataaagccatctacttttagggaaagggagttgaaaatgcaaccaactcttggcgaactgtacaaacaaatctttgctatactttatttcaaataaattctttttaaaataatttccctgcctaattatttatggaagttatgacttttgaaggacaattcaaaaccatttatttaattggttctgcaatgaaagaactgccccatatactctactaaaggcttggcactttctgctgccttttaatccagcgctataattgaggcaagcgtccagcttgacacctcgagataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 21 - protein, 418 aa, synthetic construct
  REGION 1..418: note = Recombinant protein
MYRPRPMLSPSRFFTPFAVAFVVIITVGLLAMMAGLLIHFLAFDQKSYFYRSSFQLLNVEYNSQLNSPATQEYRTLSGRIESLITKTFKESNLRNQFIRAHVAKLRQDGSGVRADVVMKFQFTRNNNGASMKSRIESVLRQMLNNSGNLEINPSTEITSLTDQAAANWLINECGAGPDLITLSEQRILGGTEAEEGSWPWQVSLRLNNAHHCGGSLINNMWILTAAHCFRSNSNPRDWIATSGISTTFPKLRMRVRNILIHNNYKSATHENDIALVRLENSVTFTKDIHSVCLPAATQNIPPGSTAYVTGWGAQEYAGHTVPELRQGQVRIISNDVCNAPHSYNGAILSGMLCAGVPQGGVDACQGDSGGPLVQEDSRRLWFIVGIVSWGDQCGLPDKPGVYTRVTAYLDWIRQQTGI

SEQ ID NO: 22 - DNA, 257 bp, synthetic construct
  misc_feature 1..257: note = synthetic oligonucleotide
  source 1..257: mol_type = other DNA
agcacccctctcttccgcagagtctaagaaatcgctgtgtttagccctcgccctgggcactgtcctcacgggagctgctgtggctgctgtcttgctttggaagttcagtaagtgcagggagcctcgatcccaccatgtgctcctgcagtccccagtgctctgagccagaccctgctctctgggctattgagacctctggaggccctccgtgaggttcctctcttacataacgaggctgtctctcttcccttctcttg

SEQ ID NO: 23 - DNA, 190 bp, synthetic construct
  misc_feature 1..190: note = synthetic oligonucleotide
  source 1..190: mol_type = other DNA
ggtcagaggaccaaaggtgaggcaaggccagacttggtgctcctgtggttctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctga

SEQ ID NO: 24 - DNA, 171 bp, synthetic construct
  misc_feature 1..171: note = synthetic oligonucleotide
  source 1..171: mol_type = other DNA
attgttttgccaagttctaattccatcagacctcgacctgcagcccctagataacttcgtataatgtatgctatacgaagttatgctagtaactataacggtcctaaggtagcgagctagctccacgtggctttgtcccagacttcctttgtcttcaacaaccttctgcaa

SEQ ID NO: 25 - DNA, 177 bp, synthetic construct
  misc_feature 1..177: note = synthetic oligonucleotide
  source 1..177: mol_type = other DNA
ggtcagaggaccaaaggtgaggcaaggccagacttggtgctcctgtggttctcgagataacttcgtataatgtatgctatacgaagttatgctagtaactataacggtcctaaggtagcgagctagctccacgtggctttgtcccagacttcctttgtcttcaacaaccttctgcaa

SEQ ID NO: 26 - DNA, 20 bp, synthetic construct
  misc_feature 1..20: note = synthetic oligonucleotide
  source 1..20: mol_type = other DNA
gccgtgactgtgaccttctc

SEQ ID NO: 27 - DNA, 22 bp, synthetic construct
  misc_feature 1..22: note = synthetic oligonucleotide
  source 1..22: mol_type = other DNA
tggaggagccacctgatgcctc

SEQ ID NO: 28 - DNA, 20 bp, synthetic construct
  misc_feature 1..20: note = synthetic oligonucleotide
  source 1..20: mol_type = other DNA
gccttgccctcaatggaaac

SEQ ID NO: 29 - DNA, 21 bp, synthetic construct
  misc_feature 1..21: note = synthetic oligonucleotide
  source 1..21: mol_type = other DNA
ggttgcacagcaaggaagaag

SEQ ID NO: 30 - DNA, 24 bp, synthetic construct
  misc_feature 1..24: note = synthetic oligonucleotide
  source 1..24: mol_type = other DNA
ccaggagttcctgtgagcctaccc

SEQ ID NO: 31 - DNA, 20 bp, synthetic construct
  misc_feature 1..20: note = synthetic oligonucleotide
  source 1..20: mol_type = other DNA
tggaatggaaggagctggag

SEQ ID NO: 32 - DNA, 19 bp, synthetic construct
  misc_feature 1..19: note = synthetic oligonucleotide
  source 1..19: mol_type = other DNA
gtcccacctcctgcaactg

SEQ ID NO: 33 - DNA, 22 bp, synthetic construct
  misc_feature 1..22: note = synthetic oligonucleotide
  source 1..22: mol_type = other DNA
tgagccttcccatcagcctggg

SEQ ID NO: 34 - DNA, 21 bp, synthetic construct
  misc_feature 1..21: note = synthetic oligonucleotide
  source 1..21: mol_type = other DNA
ccacaatggcacatgggtctg

SEQ ID NO: 35 - DNA, 18 bp, synthetic construct
  misc_feature 1..18: note = synthetic oligonucleotide
  source 1..18: mol_type = other DNA
ggtgcttgctccccaaga

SEQ ID NO: 36 - DNA, 20 bp, synthetic construct
  misc_feature 1..20: note = synthetic oligonucleotide
  source 1..20: mol_type = other DNA
cctaaaaggtgttgtaatgg

SEQ ID NO: 37 - DNA, 24 bp, synthetic construct
  misc_feature 1..24: note = synthetic oligonucleotide
  source 1..24: mol_type = other DNA
ggcaataaagaaggaagacgtttt

SEQ ID NO: 38 - DNA, 120 bp, synthetic construct
  misc_feature 1..120: note = synthetic oligonucleotide
  source 1..120: mol_type = other DNA
ccagtcagggacacacatgctcacacgcccgcccacccgcacacactacagtcgagataacttcgtataatgtatgctatacgaagttatatgcatggcctccgcgccgggttttggcgc

SEQ ID NO: 39 - DNA, 198 bp, synthetic construct
  misc_feature 1..198: note = synthetic oligonucleotide
  source 1..198: mol_type = other DNA
attctagttgtggtttgtccaaactcatcaatgtatcttatcatgtctggaataacttcgtataatgtatgctatacgaagttatgctagtaactataacggtcctaaggtagcgagctagccaagtctgtgtgctaccaagtagcaaaactgagcctggaactcacacatgcgtgtctgagagcccagcactatcgc

SEQ ID NO: 40 - DNA, 100 bp, synthetic construct
  misc_feature 1..100: note = synthetic oligonucleotide
  source 1..100: mol_type = other DNA
taatctgactttctcttcatcggtctctcttattctaggctgagctgtaacgctgccgtcccccacatccagaagctgcttcccttcagacctacctacg

SEQ ID NO: 41 - DNA, 177 bp, synthetic construct
  misc_feature 1..177: note = synthetic oligonucleotide
  source 1..177: mol_type = other DNA
ccagtcagggacacacatgctcacacgcccgcccacccgcacacactacagtcgagataacttcgtataatgtatgctatacgaagttatgctagtaactataacggtcctaaggtagcgagctagccaagtctgtgtgctaccaagtagcaaaactgagcctggaactcacacatg

SEQ ID NO: 42 - DNA, 19 bp, synthetic construct
  misc_feature 1..19: note = synthetic oligonucleotide
  source 1..19: mol_type = other DNA
gagcagggccatgacacat

SEQ ID NO: 43 - DNA, 24 bp, synthetic construct
  misc_feature 1..24: note = synthetic oligonucleotide
  source 1..24: mol_type = other DNA
accattagatcccagcactggaca

SEQ ID NO: 44 - DNA, 20 bp, synthetic construct
  misc_feature 1..20: note = synthetic oligonucleotide
  source 1..20: mol_type = other DNA
aaacccttcccgagagagaa

SEQ ID NO: 45 - DNA, 23 bp, synthetic construct
  misc_feature 1..23: note = synthetic oligonucleotide
  source 1..23: mol_type = other DNA
gaggaacactgtgtcaaggactt

SEQ ID NO: 46 - DNA, 22 bp, synthetic construct
  misc_feature 1..22: note = synthetic oligonucleotide
  source 1..22: mol_type = other DNA
cctgaaaagcccggagtggcag

SEQ ID NO: 47 - DNA, 19 bp, synthetic construct
  misc_feature 1..19: note = synthetic oligonucleotide
  source 1..19: mol_type = other DNA
gggcagagaccacatctga

SEQ ID NO: 48 - DNA, 22 bp, synthetic construct
  misc_feature 1..22: note = synthetic oligonucleotide
  source 1..22: mol_type = other DNA
ggaagccctctctcgatacttg

SEQ ID NO: 49 - DNA, 22 bp, synthetic construct
  misc_feature 1..22: note = synthetic oligonucleotide
  source 1..22: mol_type = other DNA
ttctaccctgagggcatgcagc

SEQ ID NO: 50 - DNA, 22 bp, synthetic construct
  misc_feature 1..22: note = synthetic oligonucleotide
  source 1..22: mol_type = other DNA
tgggatgtagaaggttgtcaga

SEQ ID NO: 51 - DNA, 22 bp, synthetic construct
  misc_feature 1..22: note = synthetic oligonucleotide
  source 1..22: mol_type = other DNA
ctgagcctggaactcacacatg

SEQ ID NO: 52 - DNA, 23 bp, synthetic construct
  misc_feature 1..23: note = synthetic oligonucleotide
  source 1..23: mol_type = other DNA
tctgagagcccagcactatcgcc

SEQ ID NO: 53 - DNA, 19 bp, synthetic construct
  misc_feature 1..19: note = synthetic oligonucleotide
  source 1..19: mol_type = other DNA
gctgagggtcaggcttgag

SEQ ID NO: 54 - DNA, 21 bp, synthetic construct
  misc_feature 1..21: note = synthetic oligonucleotide
  source 1..21: mol_type = other DNA
tctgcagggtagggagagaag

SEQ ID NO: 55 - DNA, 29 bp, synthetic construct
  misc_feature 1..29: note = synthetic oligonucleotide
  source 1..29: mol_type = other DNA
tgtttcagaaaaggaagactcacgttaca

SEQ ID NO: 56 - DNA, 24 bp, synthetic construct
  misc_feature 1..24: note = synthetic oligonucleotide
  source 1..24: mol_type = other DNA
gagaccgatgaagagaaagtcaga

SEQ ID NO: 57 - DNA, 100 bp, synthetic construct
  misc_feature 1..100: note = synthetic oligonucleotide
  source 1..100: mol_type = other DNA
gaccattttaaggttttgcttggttgttttggagggagggtggtgctttgctaatggtgaattactaactcctcaataaagaatattatttgaaataatt

SEQ ID NO: 58 - DNA, 190 bp, synthetic construct
  misc_feature 1..190: note = synthetic oligonucleotide
  source 1..190: mol_type = other DNA
gctgccttttaatccagcgctataattgaggcaagcgtccagcttgacacctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctga

SEQ ID NO: 59 - DNA, 171 bp, synthetic construct
  misc_feature 1..171: note = synthetic oligonucleotide
  source 1..171: mol_type = other DNA
attgttttgccaagttctaattccatcagacctcgacctgcagcccctagataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 60 - DNA, 177 bp, synthetic construct
  misc_feature 1..177: note = synthetic oligonucleotide
  source 1..177: mol_type = other DNA
ccagtcagggacacacatgctcacacgcccgcccacccgcacacactacactcgagataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 61 - DNA, 21 bp, synthetic construct
  misc_feature 1..21: note = synthetic oligonucleotide
  source 1..21: mol_type = other DNA
tcctctccagacaagaaagct

SEQ ID NO: 62 - DNA, 30 bp, synthetic construct
  misc_feature 1..30: note = synthetic oligonucleotide
  source 1..30: mol_type = other DNA
tcatagcagctttcaaatcctaaacgttga

SEQ ID NO: 63 - DNA, 20 bp, synthetic construct
  misc_feature 1..20: note = synthetic oligonucleotide
  source 1..20: mol_type = other DNA
tcgtgtgtagctggtgagtt

SEQ ID NO: 64 - DNA, 22 bp, synthetic construct
  misc_feature 1..22: note = synthetic oligonucleotide
  source 1..22: mol_type = other DNA
catgcgatcacaggaggagatc

SEQ ID NO: 65 - DNA, 22 bp, synthetic construct
  misc_feature 1..22: note = synthetic oligonucleotide
  source 1..22: mol_type = other DNA
aattgggcccgaagccagatgc

SEQ ID NO: 66 - DNA, 20 bp, synthetic construct
  misc_feature 1..20: note = synthetic oligonucleotide
  source 1..20: mol_type = other DNA
cggaaggcttctgtgacttc

SEQ ID NO: 67 - DNA, 25 bp, synthetic construct
  misc_feature 1..25: note = synthetic oligonucleotide
  source 1..25: mol_type = other DNA
gtctcccacttctgacataatgaac

SEQ ID NO: 68 - DNA, 27 bp, synthetic construct
  misc_feature 1..27: note = synthetic oligonucleotide
  source 1..27: mol_type = other DNA
cccagtgttaaccctacatctggttcc

SEQ ID NO: 69 - DNA, 20 bp, synthetic construct
  misc_feature 1..20: note = synthetic oligonucleotide
  source 1..20: mol_type = other DNA
tgggaagagactcttggaca

SEQ ID NO: 70 - DNA, 25 bp, synthetic construct
  misc_feature 1..25: note = synthetic oligonucleotide
  source 1..25: mol_type = other DNA
atgagctcctagtacagctaaagtt

SEQ ID NO: 71 - DNA, 26 bp, synthetic construct
  misc_feature 1..26: note = synthetic oligonucleotide
  source 1..26: mol_type = other DNA
atgcatgatcatctatgcgtcagagc

SEQ ID NO: 72 - DNA, 21 bp, synthetic construct
  misc_feature 1..21: note = synthetic oligonucleotide
  source 1..21: mol_type = other DNA
tgcccagatgcagggagttag

(19) United States
(12) Patent Application Publication
(10) Pub. No.: US 2024/0147971 A1
(43) Pub. Date: May 9, 2024

(54) RODENTS HAVING A HUMANIZED TMPRSS GENE

(71) Applicant: Regeneron Pharmaceuticals, Inc., Tarrytown, NY (US)

(72) Inventors: Lisa Purcell, Garnerville, NY (US); Alexander O. Mujica, Elmsford, NY (US); Yajun Tang, White Plains, NY (US)

(73) Assignee: Regeneron Pharmaceuticals, Inc., Tarrytown, NY (US)

(21) Appl. No.: 18/413,096

(22) Filed: Jan. 16, 2024

Related U.S. Application Data
(63) Continuation of application No. 17/099,942, filed on Nov. 17, 2020, now Pat. No. 11,910,787.
Application No. 17/099,942 is a division of application No. 16/052,700, filed on Aug. 2, 2018, now Pat. No. 10,863,729.
Application No. 16/052,700 is a continuation of application No. 15/442,857, filed on Feb. 27, 2017, now Pat. No. 10,070,632.
(60) Provisional application No. 62/301,023, filed on Feb. 29, 2016.

(51) Int. Cl. A01K 67/0275; A01K 67/0278; C07K 14/47; C12N 5/0735; C12N 9/64; C12N 15/63; C12N 15/86

(52) U.S. Cl. CPC A01K 67/0275; A01K 67/0278; C07K 14/47; C12N 5/0606; C12N 9/6424; C12N 15/63; C12N 15/86; A01K 2207/15; A01K 2217/072; A01K 2227/10; A01K 2227/105; A01K 2267/0337; A61K 48/00; C07K 2319/00

(57) ABSTRACT

Genetically modified rodents such as mice and rats, and methods and compositions for making and using the same, are provided. The rodents comprise a humanization of at least one endogenous rodent Tmprss gene, such as an endogenous rodent Tmprss2, Tmprss4, or Tmprss11d gene.

### CROSS REFERENCE TO RELATED APPLICATIONS

This application is a continuation of U.S. patent application Ser. No. 17/099,942, filed Nov. 17, 2020, which is a divisional of U.S. patent application Ser. No. 16/052,700, filed Aug. 2, 2018, now U.S. Pat. No. 10,863,729, which is a continuation of U.S. patent application Ser. No. 15/442,857, filed Feb. 27, 2017, now U.S. Pat. No. 10,070,632, which claims the benefit of priority from U.S. Provisional Application No. 62/301,023, filed Feb. 29, 2016, the entire contents of which are incorporated herein by reference.

### INCORPORATION BY REFERENCE OF SEQUENCE LISTING

The Sequence Listing in the XML file, named as 33093YAZ_10234US05_SequenceListing.xml of 266 KB, created on Dec. 21, 2023, and submitted to the United States Patent and Trademark Office via Patent Center, is incorporated herein by reference.

### BACKGROUND

Type II transmembrane serine proteases are a family of proteases characterized by an N-terminal transmembrane domain (Bugge et al., J. Biol. Chem. 284 (35): 23177-23181, 2009; Hooper et al., J. Biol. Chem. 272(2): 857-860, 2001). All members of this family are expressed as single-chain zymogens and are proteolytically activated by cleavage within a highly conserved R/(IV)VGG motif. One member of the family, transmembrane protease, serine type 4 (TMPRSS4), has been shown to activate the epithelial sodium channel (ENaC) regulating the sodium and water flux across epithelia (Guipponi et al. 2002 Hum. Mol. Genet. 11:2829; Vuagniaux et al. 2002 J. Gen. Physiol. 120:191). The proteolytical activators of TMPRSS4 are unknown; however, data available to date suggests that the protein is autoactivated. When activated, the catalytic domain of TMPRSS4 remains bound to the N-terminus of the protein via a disulphide linkage. TMPRSS4, TMPRSS2 and TMPRSS 11D (or Human Airway Trypsin-like protease; “HAT”) have been shown in vitro to cleave influenza A hemagglutinin (HA), which is the first essential step in the viral life cycle. This cleavage is essential for activity of HA, as the protein is synthesized as a precursor protein (HA0) and requires cleavage into HA1 and HA2 for activity. RNAi knock-down of TMPRSS4 in Caco-2 cells resulted in reduced spread of the virus. In addition, TMPRSS4 was shown to be strongly upregulated in the lungs of mice infected with influenza (Bottcher et al. 2006 J. Virol. 80:9896; Bottcher et al. 2009 Vaccine 27: 6324; Bottcher-Friebershausser et al. 2010 J. Virol. 84: 5604; Bertam et al. 2010 J. Virol. 84:10016; Bertam et al. 2010 J. Virol. 84:10016; Bottcher-Friebershausser et al. 2011 J. Virol. 85: 1554; Bahgat et al. 2011 Virol. J. 8:27).

Development of an in vivo system, e.g., a rodent model of infection, is needed in order to identify and test compounds including antibodies that specifically target human type II transmembrane serine proteases for the treatment and prevention of viral infection and other diseases.

### SUMMARY

The present invention encompasses the recognition that it is desirable to engineer rodent animals to provide in vivo systems for identifying and developing new therapeutics. For example, the present invention encompasses the recognition that rodents having a humanized Tmprss gene are desirable for use in identifying and developing therapeutics for the treatment and prevention of viral infections.

In one aspect, the invention provides a rodent whose genome contains a humanized Tmprss gene that includes a nucleotide sequence of an endogenous rodent Tmprss gene and a nucleotide sequence of a cognate human TMPRSS gene, wherein the humanized Tmprss gene is under control of a 5′ regulatory sequence(s), such as the promoter and/or enhancer(s), of the endogenous rodent Tmprss gene.

In some embodiments, the humanized Tmprss gene in rodents disclosed herein encodes a humanized Tmprss protein that contains an ectodomain substantially identical (e.g., at least 85%, 90%, 95%, 98%, 99% or 100% identical in sequence) to the ectodomain of a human TMPRSS protein. In some embodiments, the humanized Tmprss protein contains a cytoplasmic and transmembrane portion that is substantially identical (e.g., at least 85%, 90%, 95%, 98%, 99% or 100% identical in sequence) to the cytoplasmic and transmembrane portion of an endogenous rodent Tmprss protein.

In some embodiments, a rodent disclosed herein contains a humanized Tmprss gene that includes a nucleotide sequence of an endogenous rodent Tmprss gene and a nucleotide sequence of a cognate human TMPRSS gene, wherein the nucleotide sequence of the cognate human TMPRSS gene encodes a polypeptide substantially identical (e.g., at least 85%, 90%, 95%, 98%, 99% or 100% identical in sequence) to the ectodomain of the human TMPRSS protein encoded by the cognate human TMPRSS gene. In some embodiments, a rodent disclosed herein contains a humanized Tmprss gene that includes a nucleotide sequence of an endogenous rodent Tmprss gene and a nucleotide sequence of a cognate human TMPRSS gene, wherein the nucleotide sequence of the endogenous rodent Tmprss gene encodes a polypeptide substantially identical (e.g., at least 85%, 90%, 95%, 98%, 99% or 100% identical in sequence) to the cytoplasmic and transmembrane portion of the endogenous rodent Tmprss protein encoded by the endogenous rodent Tmprss gene.

In some embodiments, a rodent disclosed herein contains a humanized Tmprss gene located at an endogenous rodent Tmprss locus that results from a replacement of a contiguous genomic sequence of an endogenous rodent Tmprss gene with a contiguous genomic sequence of a cognate human TMPRSS gene. In specific embodiments, the contiguous genomic sequence of a cognate human TMPRSS gene being inserted includes exon sequences encoding an ectodomain substantially identical with the ectodomain of the human TMPRSS protein encoded by human TMPRSS gene. In some embodiments, the contiguous genomic sequence of a cognate human TMPRSS gene also includes the 3′ UTR of the cognate human TMPRSS gene.

In some embodiments, a rodent disclosed herein is heterozygous for a humanized Tmprss gene at an endogenous rodent Tmprss locus. In other embodiments, a rodent is homozygous for a humanized Tmprss gene at an endogenous rodent Tmprss locus.

In further embodiments, a rodent contains two or more humanized Tmprss genes at different endogenous rodent Tmprss loci with each endogenous rodent Tmprss locus being humanized with a respective cognate human TMPRSS gene; for example, two or more of humanized Tmprss2, humanized Tmprss4, and humanized Tmprss11d genes.

In some embodiments, a rodent disclosed herein contains a humanized Tmprss2 gene that includes a nucleotide sequence of an endogenous rodent Tmprss2 gene and a nucleotide sequence of a human TMPRSS2 gene, wherein the humanized Tmprss2 gene is under control of the promoter of the endogenous rodent Tmprss2 gene.

In some embodiments, the humanized Tmprss2 gene encodes a humanized Tmprss2 protein that contains an ectodomain substantially identical (e.g., at least 85%, 90%, 95%, 98%, 99% or 100% identical in sequence) with the ectodomain of the human TMPRSS2 protein encoded by the human TMPRSS2 gene used in humanization. The human TMPRSS2 protein contains, in some embodiments, an amino acid sequence at least 85% identical (e.g., at least 90%, 95%, 98%, 99% or 100% identical) with the amino acid sequence as set forth in SEQ ID NO: 4. In some embodiments, a humanized Tmprss2 protein contains an ectodomain substantially identical (e.g., at least 85%, 90%, 95%, 98%, 99% or 100% identical) with the amino acid sequence composed of residues W106 to G492 or the C-terminal 387 amino acids of a human TMPRSS2 protein as set forth in, e.g., SEQ ID NO: 4. In some embodiments, the humanized Tmprss2 gene encodes a humanized Tmprss2 protein that further contains a cytoplasmic and transmembrane portion that is substantially identical (e.g., at least 85%, 90%, 95%, 98%, 99% or 100% identical) with the cytoplasmic and transmembrane portion of the rodent Tmprss2 protein encoded by the endogenous rodent Tmprss2 gene being humanized. An exemplary endogenous rodent Tmprss2 protein is set forth in SEQ ID NO: 2.

In some embodiments, a rodent contains a humanized Tmprss2 gene that includes a nucleotide sequence of an endogenous rodent Tmprss2 gene and a nucleotide sequence of a human TMPRSS2 gene, wherein the nucleotide sequence of the human TMPRSS2 gene encodes an ectodomain substantially identical (e.g., at least 85%, 90%, 95%, 98%, 99% or 100% identical in sequence) with the ectodomain of the human TMPRSS2 protein encoded by the human TMPRSS2 gene. In specific embodiments, the nucleotide sequence of a human TMPRSS2 gene is a contiguous genomic sequence of a human TMPRSS2 gene containing coding exon 4 through the stop codon in coding exon 13 of the human TMPRSS2 gene. In particular embodiments, the contiguous genomic sequence of a human TMPRSS2 gene further contains the 3′ UTR of the human TMPRSS2 gene. In some embodiments, the nucleotide sequence of an endogenous rodent Tmprss2 gene included in a humanized Tmprss2 gene encodes a cytoplasmic and transmembrane portion that is substantially identical (e.g., at least 85%, 90%, 95%, 98%, 99% or 100% identical) with the cytoplasmic and transmembrane portion of the endogenous rodent Tmprss2 protein encoded by the endogenous rodent Tmprss2 gene.

In particular embodiments, a humanized Tmprss2 gene contains coding exons 1-2 of an endogenous rodent Tmprss2 gene, and coding exon 4 through coding exon 13 of a human TMPRSS2 gene, wherein the humanized Tmprss2 gene encodes a humanized Tmprss2 protein that contains a cytoplasmic and transmembrane portion that is substantially identical with the cytoplasmic and transmembrane portion of the rodent Tmprss2 protein encoded by the endogenous rodent Tmprss2 gene, and an ectodomain that is substantially identical with the ectodomain of the human TMPRSS2 protein encoded by the human TMPRSS2 gene. The humanized Tmprss2 gene contains an exon 3 that in some embodiments is coding exon 3 of a human TMPRSS2 gene, and in other embodiments is coding exon 3 of an endogenous rodent Tmprss2 gene. In some embodiments, the humanized Tmprss2 gene contains an exon 3 that includes a 5′ portion of coding exon 3 of an endogenous rodent Tmprss2 gene and a 3′ portion of coding exon 3 of a human TMPRSS2 gene.

In some embodiments, a rodent disclosed herein contains a humanized Tmprss4 gene that includes a nucleotide sequence of an endogenous rodent Tmprss4 gene and a nucleotide sequence of a human TMPRSS4 gene, wherein the humanized Tmprss4 gene is under control of the promoter of the endogenous rodent Tmprss4 gene.

In some embodiments, the humanized Tmprss4 gene encodes a humanized Tmprss4 protein that contains an ectodomain substantially identical (e.g., at least 85%, 90%, 95%, 98%, 99% or 100% identical in sequence) with the ectodomain of the human TMPRSS4 protein encoded by the human TMPRSS4 gene used in humanization. The human TMPRSS4 protein contains, in some embodiments, an amino acid sequence at least 85% identical (e.g., at least 90%, 95%, 98%, 99% or 100% identical) with the amino acid sequence as set forth in SEQ ID NO: 11. In some embodiments, a humanized Tmprss4 protein contains an ectodomain substantially identical (e.g., at least 85%, 90%, 95%, 98%, 99% or 100% identical) with the amino acid sequence composed of residues K54 to L437 or the C-terminal 384 amino acids of a human TMPRSS4 protein as set forth in, e.g., SEQ ID NO: 11. In some embodiments, the humanized Tmprss4 gene encodes a humanized Tmprss4 protein that further contains a cytoplasmic and transmembrane portion that is substantially identical (e.g., at least 85%, 90%, 95%, 98%, 99% or 100% identical) with the cytoplasmic and transmembrane portion of the rodent Tmprss4 protein encoded by the endogenous rodent Tmprss4 gene being humanized. An exemplary endogenous rodent Tmprss4 protein is set forth in SEQ ID NO: 9.

In some embodiments, a rodent contains a humanized Tmprss4 gene that includes a nucleotide sequence of an endogenous rodent Tmprss4 gene and a nucleotide sequence of a human TMPRSS4 gene, wherein the nucleotide sequence of a human TMPRSS4 gene encodes an ectodomain substantially identical with the ectodomain of the human TMPRSS4 protein encoded by the human TMPRSS4 gene. In specific embodiments, the nucleotide sequence of a human TMPRSS4 gene is a contiguous genomic sequence containing coding exon 4 through the stop codon in coding exon 13 of a human TMPRSS4 gene. In some embodiments, the nucleotide sequence of an endogenous rodent Tmprss4 gene included in a humanized Tmprss4 gene encodes a cytoplasmic and transmembrane portion that is substantially identical with the cytoplasmic and transmembrane portion of the rodent Tmprss4 protein encoded by the endogenous rodent Tmprss4 gene.

In particular embodiments, a humanized Tmprss4 gene contains coding exon 1 through coding exon 3 of an endogenous rodent Tmprss4 gene, and coding exon 4 through the stop codon in coding exon 13 of a human TMPRSS4 gene.

In some embodiments, a rodent disclosed herein contains a humanized Tmprss11d gene that includes a nucleotide sequence of an endogenous rodent Tmprss11d gene and a nucleotide sequence of a human TMPRSS11D gene, wherein the humanized Tmprss11d gene is under control of the promoter of the endogenous rodent Tmprss11d gene.

In some embodiments, the humanized Tmprss11d gene encodes a humanized Tmprss11d protein that contains an ectodomain substantially identical (e.g., at least 85%, 90%, 95%, 98%, 99% or 100% identical in sequence) with the ectodomain of the human TMPRSS11D protein encoded by the human TMPRSS11D gene used in humanization. The human TMPRSS1ID protein contains, in some embodiments, an amino acid sequence at least 85% identical (e.g., at least 90%, 95%, 98%, 99% or 100% identical) with the amino acid sequence as set forth in SEQ ID NO: 18. In some embodiments, a humanized Tmprss11d protein contains an ectodomain substantially identical (e.g., at least 85%, 90%, 95%, 98%, 99% or 100% identical) with the amino acid sequence composed of residues A42-I418 or the C-terminal 377 amino acids of a human TMPRSS11D protein as set forth in, e.g., SEQ ID NO: 18. In some embodiments, the humanized Tmprss11d gene encodes a humanized Tmprss11d protein that further contains a cytoplasmic and transmembrane portion that is substantially identical (e.g., at least 85%, 90%, 95%, 98%, 99% or 100% identical) with the cytoplasmic and transmembrane portion of the endogenous rodent Tmprss11d protein encoded by the endogenous rodent Tmprss11d gene being humanized. An exemplary endogenous rodent Tmprss11d protein is set forth in SEQ ID NO: 16.

In some embodiments, a rodent contains a humanized Tmprss11d gene that includes a nucleotide sequence of an endogenous rodent Tmprss11d gene and a nucleotide sequence of a human TMPRSS11D gene, wherein the nucleotide sequence of the human TMPRSS11D gene encodes an ectodomain substantially identical with the ectodomain of the human TMPRSS11D protein encoded by the human TMPRSS11D gene. In specific embodiments, the nucleotide sequence of a human TMPRSS11d gene is a contiguous genomic sequence containing coding exon 3 through the stop codon in coding exon 10 of a human TMPRSS11D gene. In particular embodiments, the contiguous genomic sequence of a human TMPRSS11D gene further contains the 3′ UTR of the human TMPRSS11D gene. In some embodiments, the nucleotide sequence of an endogenous rodent Tmprss11d gene included in a humanized Tmprss11d gene encodes a cytoplasmic and transmembrane portion that is substantially identical with the cytoplasmic and transmembrane portion of the rodent Tmprss11d protein encoded by the endogenous rodent Tmprss11d gene.

In particular embodiments, a humanized Tmprss11d gene contains coding exons 1-2 of an endogenous rodent Tmprss11d gene, and coding exon 3 through coding exon 13 of a human TMPRSS11D gene.

In another aspect, the invention provides an isolated rodent cell or tissue whose genome contains a humanized Tmprss gene as described herein. In specific embodiments, the humanized Tmprss gene is selected from the group consisting of a humanized Tmprss2 gene, a humanized Tmprss4 gene, and a humanized Tmprss11d gene.

In still another aspect, the invention provides a rodent embryonic stem cell whose genome contains a humanized Tmprss gene as described herein. In specific embodiments, the humanized Tmprss gene is selected from the group consisting of a humanized Tmprss2 gene, a humanized Tmprss4 gene, and a humanized Tmprss11d gene.

In another aspect, a rodent embryo generated from the rodent embryonic stem cell disclosed herein is also provided.

In one aspect, the invention provides a nucleic acid vector suitable for use in humanizing an endogenous Tmprss gene in a rodent. In some embodiments, the nucleic acid vector includes a human Tmprss nucleic acid sequence (e.g., a human genomic DNA encoding the ectodomain of a human TMPRSS protein), flanked by a 5′ homology arm and a 3′ homology arm. The 5′ and 3′ homology arms are nucleic acid sequences that are placed at 5′ and 3′, respectively, to the human Tmprss nucleic acid sequence and are homologous to genomic DNA sequences at an endogenous Tmprss locus in a rodent that flank a rodent genomic DNA encoding the ectodomain of a cognate rodent Tmprss protein. Thus, the 5′ and 3′ homology arms are capable of mediating homologous recombination and replacement of the rodent genomic DNA encoding the ectodomain of the cognate rodent Tmprss protein with the human Tmprss nucleic acid sequence to form a humanized Tmprss gene as described herein.

In a further aspect, the invention is directed to a method of providing a rodent whose genome contains a humanized Tmprss gene. The method includes modifying the genome of a rodent to replace a genomic sequence of an endogenous rodent Tmprss gene with a genomic sequence of a cognate human TMPRSS gene to form a humanized Tmprss gene.

In some embodiments, the invention provides a method of making a rodent (such as a mouse or a rat) having a humanized Tmprss gene, the method including the steps of (a) inserting a genomic fragment into an endogenous rodent Tmprss locus in a rodent embryonic stem cell, wherein the genomic fragment contains a nucleotide sequence of a cognate human TMPRSS gene, thereby forming a humanized Tmprss gene (such as those described herein); (b) obtaining a rodent embryonic stem cell comprising the humanized Tmprss gene of (a); and (c) creating a rodent using the rodent embryonic stem cell of (b).

In some embodiments, the humanized Tmprss gene is selected from the group consisting of a humanized Tmprss2 gene, a humanized Tmprss4 gene, and a humanized Tmprss11d gene. In various embodiments, the humanized Tmprss gene encodes a humanized Tmprss protein that contains an ectodomain substantially identical (e.g., at least 90%, 95%, 98%, 99% or 100% identical in sequence) to the ectodomain of the human TMPRSS protein encoded by the human TMPRSS gene used for humanization. In specific embodiments, the humanized Tmprss protein contains the ectodomain of a human TMPRSS protein selected from the group consisting of a human TMPRSS2 protein, a human TMPRSS4 protein, and a human TMPRSS11D protein. In specific embodiments, the humanized Tmprss protein further contains a cytoplasmic and transmembrane portion that is substantially identical with the cytoplasmic and transmembrane portion of the rodent Tmprss protein encoded by the endogenous rodent Tmprss gene being humanized.

In another aspect, the invention provides a method of using a rodent disclosed herein to assess the therapeutic efficacy of a compound (e.g., candidate inhibitors that specifically target a human TMPRSS protein) in treating influenza virus infection. The method can include the steps of providing a rodent described herein, administering an influenza virus and a candidate compound to the rodent; and monitoring the presence and severity of influenza virus infection in the rodent to determine the therapeutic efficacy of the drug candidate.

In some embodiments, the influenza virus is administered to the rodent before the compound. In other embodiments, the influenza virus is administered to the rodent after the compound.

In some embodiments, the candidate compound is an antibody or antigen-binding fragment thereof specific for a human TMPRSS protein. In specific embodiments, the candidate compound is an antibody or antigen-binding fragment thereof specific for a human TMPRSS protein selected from the group consisting of a human TMPRSS2 protein, a human TMPRSS4 protein, and a human TMPRSS11D protein.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The Drawings included herein, which are composed of the following Figures, are for illustration purposes only and not for limitation.

FIGS. 1A-1D. Exemplary strategy for humanization of mouse Tmprss2.

FIG. 1A shows a diagram, not to scale, of the genomic organization of mouse Tmprss2 and human TMPRSS2 genes. Exons are represented by thin bars placed across the genomic sequences, with the first coding exon for both genes indicated by the start codon “ATG” above the exon, and the last coding exon indicated by the “Stop” codon above the exon. A mouse genomic fragment of about 25,291 bp to be deleted and a human genomic fragment of about 25,091 bp to be inserted are indicated. Locations of probes used in an assay described in Example 1 are indicated. TM: transmembrane domain; SRCR: scavenger receptor cysteine-rich like domain; LDLRa: low density lipoprotein receptor class A.

FIG. 1B illustrates, not to scale, an exemplary modified BAC vector for humanization of an endogenous mouse Tmprss2 gene, along with the junction sequences (SEQ ID NOS: 22, 23 and 24).

FIG. 1C illustrates, not to scale, a humanized Tmprss2 allele after the neomycin cassette has been deleted, along with the junction sequences (SEQ ID NOS: 22 and 25).

FIG. 1D sets forth a sequence alignment of a human TMPRSS2 protein (SEQ ID NO: 4), a mouse Tmprss2 protein (SEQ ID NO: 2), and a humanized Tmprss2 protein (“7010 mutant pro”) (SEQ ID NO: 7).

FIGS. 2A-2D. Exemplary strategy for humanization of mouse Tmprss4.

FIG. 2A shows a diagram, not to scale, of the genomic organization of mouse Tmprss4 and human TMPRSS4 genes. Exons are represented by thin bars placed across the genomic sequences, with the first exon (also the first coding exon) for both genes indicated by the start codon “ATG” above the exon, and the last coding exon indicated by the “Stop” codon above the exon. The mouse genomic fragment of about 11,074 bp to be deleted and the human genomic fragment of about 14,963 bp to be inserted are indicated. Locations of probes used in an assay described in Example 2 are indicated. TM: transmembrane domain; SRCR: scavenger receptor cysteine-rich like domain; LDLRa: low density lipoprotein receptor class A.

FIG. 2B illustrates, not to scale, an exemplary modified BAC vector for humanization of an endogenous mouse Tmprss4 gene, along with the junction sequences (SEQ ID NOS: 38, 39 and 40).

FIG. 2C illustrates, not to scale, a humanized Tmprss4 allele after the neomycin cassette has been deleted, along with the junction sequences (SEQ ID NOS: 41 and 40).

FIG. 2D sets forth a sequence alignment of a human TMPRSS4 protein (SEQ ID NO: 11), a mouse Tmprss4 protein (SEQ ID NO: 9), and a humanized Tmprss4 protein (“7224 mutant pro”) (SEQ ID NO: 14).

FIGS. 3A-3D. Exemplary strategy for humanization of mouse Tmprss11d.

FIG. 3A shows a diagram, not to scale, of the genomic organization of mouse Tmprss11d and human TMPRSS11D genes. Exons are represented by thin bars placed across the genomic sequences, with the first exon (also the first codon exon) for both genes indicated by the start codon “ATG” above the exon, and the last coding exon indicated by the “Stop” codon above the exon. A mouse genomic fragment of about 35,667 bp to be deleted and a human genomic fragment of about 33,927 bp to be inserted are indicated. Locations of probes used in an assay described in Example 3 are indicated. TM: transmembrane domain; SEA: domain found in sea urchin sperm protein, enterokinase and agrin.

FIG. 3B illustrates, not to scale, an exemplary modified BAC vector for humanization of an endogenous mouse Tmprss11d gene, along with the junction sequences (SEQ ID NOS: 57, 58 and 59).

FIG. 3C illustrates, not to scale, a humanized Tmprss11 allele after the neomycin cassette has been deleted, along with the junction sequences (SEQ ID NOS: 57 and 60).

FIG. 3D sets forth a sequence alignment of a human TMPRSS11D protein (SEQ ID NO: 18), a mouse Tmprss11d protein (SEQ ID NO: 16), and a humanized Tmprss11d protein (“7226 mutant pro”) (SEQ ID NO: 21).

FIG. 4 depicts the results of an experiment showing that MAID7225 HumInTMPRSS4 mice do not differ in their susceptibility to challenge with high doses of severe influenza A H1N1 or severe, mouse-adapted H3N2. MAID7225 HumIn TMRPSS4 mice challenged with A/Puerto Rico/08/1934 (H1N1) (light gray circles, dotted line) showed similar survival rates compared to wild-type mice (light gray squares, dotted line). Likewise, MAID7225 HumIn TMRPSS4 mice challenged with A/Aichi/02/1968-X31 (H3N2) (dark gray triangles, dotted line) showed similar survival rates compared to wild-type mice (light gray inverse triangles, dashed line). Mice were infected IN on day 0 with either 1150 PFUs of A/Puerto Rico/08/1934 (H1N1) or 10,000 PFUs of A/Aichi/02/1968-X31 (H3N2). The control group included uninfected negative control MAID7225 HumIn TMPRSS4 and wild-type mice (black diamonds, solid line).

### DETAILED DESCRIPTION OF CERTAIN EMBODIMENTS

The present invention relates to genetically modified rodents (e.g., mice and rats) having a humanized gene encoding a type II transmembrane serine protease (or “Tmprss”, for transmembrane protease/serine). The genetically modified rodents are suitable for use in screening for candidate compounds that specifically target a human TMPRSS molecule for the treatment and prevention of diseases such as influenza virus infection. Accordingly, the present invention provides genetically modified rodents having a humanized Tmprss gene, cells and tissues isolated from the genetically modified rodents, methods and compositions for making the genetically modified rodents, and use of the genetically modified rodents for screening and testing therapeutic compounds. The various embodiments of the present invention are further described below.

### Type II Transmembrane Serine Proteases (“Tmprss”)

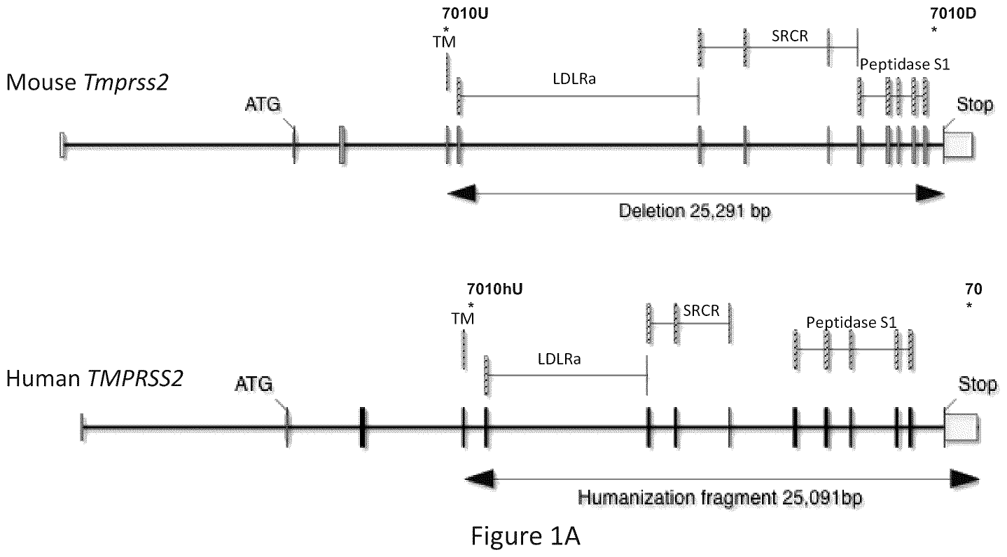

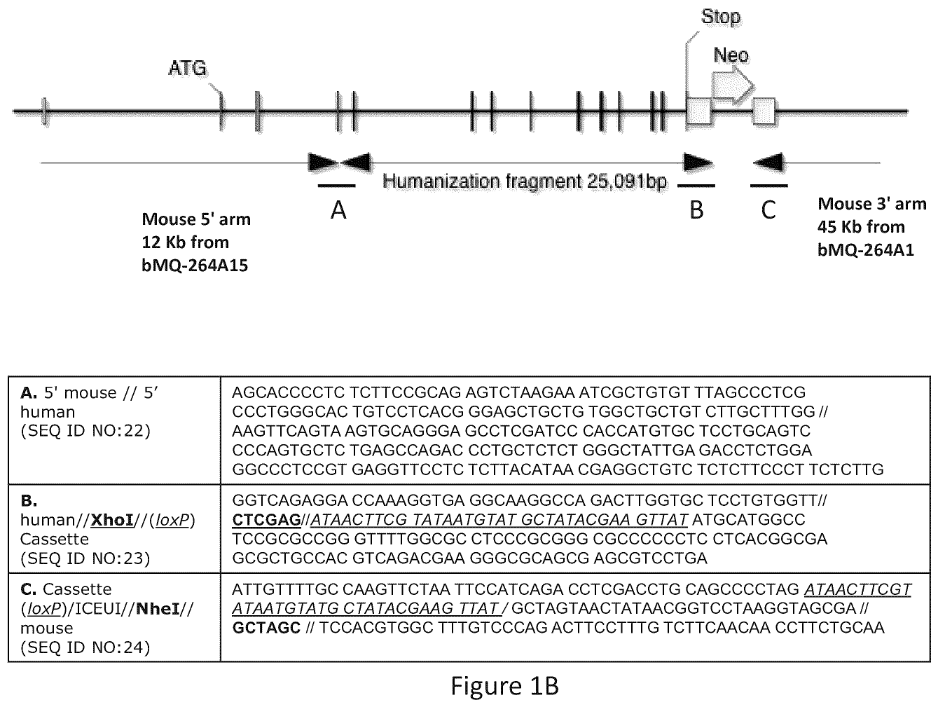

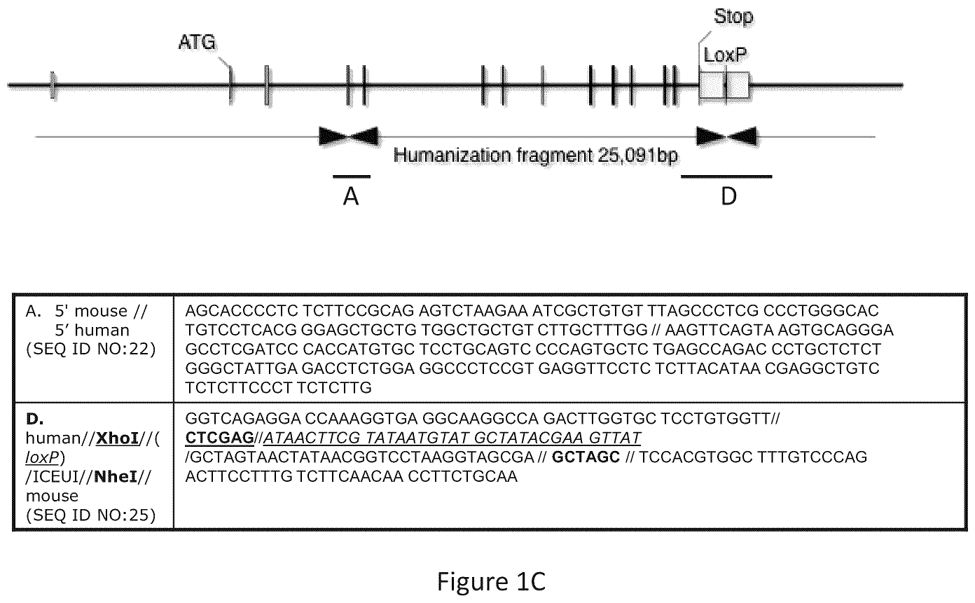

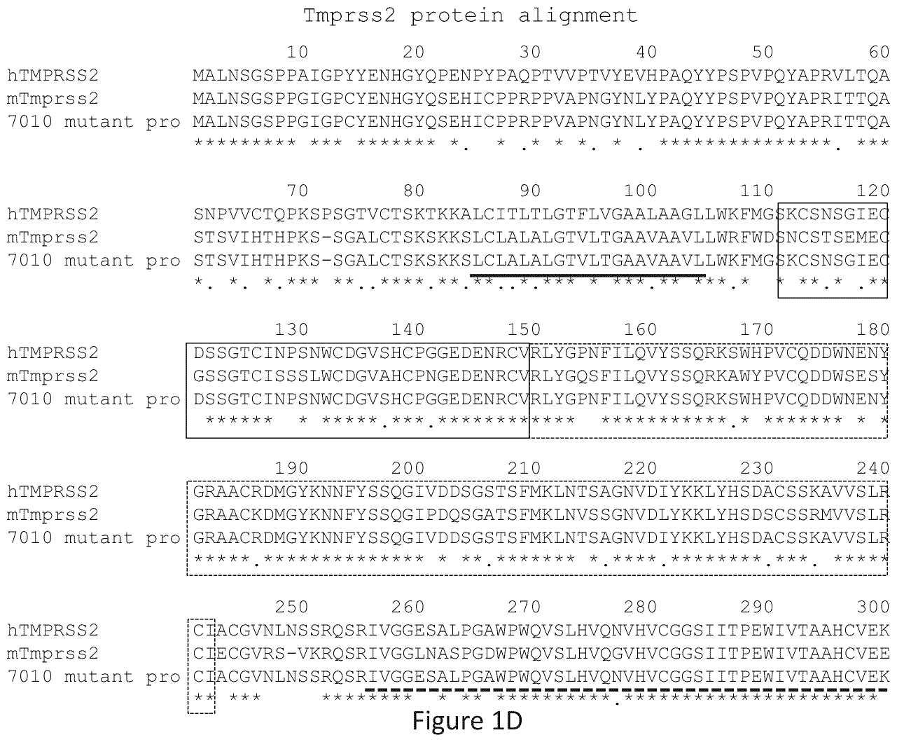

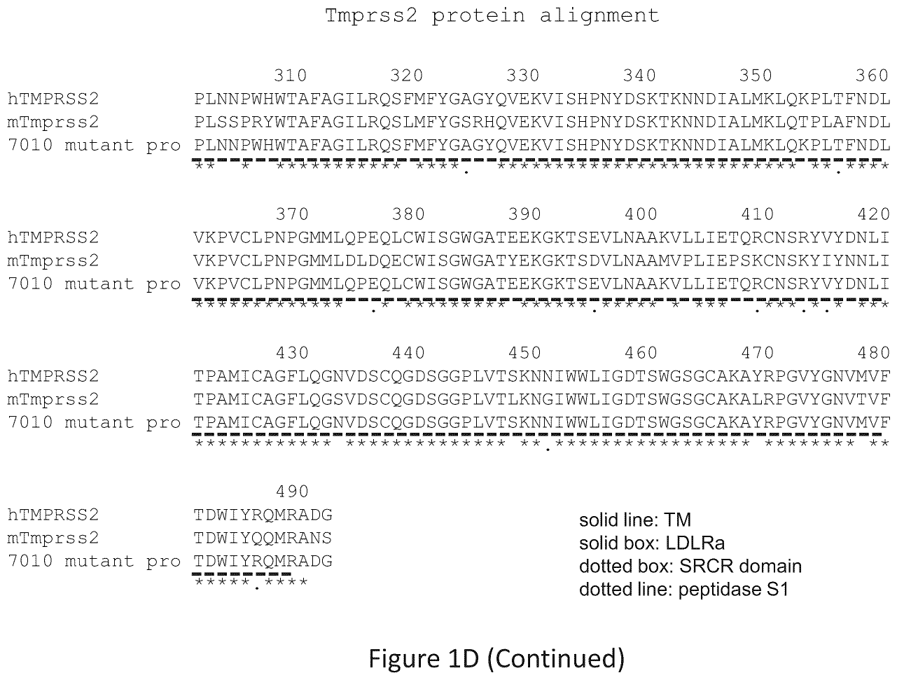

Type II transmembrane serine proteases, also referred to herein as “Tmprss” for non-human molecules or “TMPRSS” for human molecules (“transmembraneprotease/serine”), are a family of proteins characterized by an N-terminal transmembrane domain and a C-terminal extracellular serine protease domain. At least 18 members have been identified in the family, which are grouped into four subfamilies (Bugge et al. (2009), supra). All members share several common structural features that define the family, including (i) a short N-terminal cytoplasmic domain, (ii) a transmembrane domain, and (iii) an ectodomain that contains a protease domain and a stem region that links the transmembrane domain with the protease domain. The stem region contains a combination of modular structural domains of six different types: a SEA (sea urchin sperm protein/enteropeptidase/agrin) domain, a group A scavenger receptor domain, a LDLA (low-density lipoprotein receptor class A) domain, a CUB (Cls/Clr urchin embryonic growth factor, bone morphogenetic protein-1) domain, a MAM (meprin/A5 antigen/receptor protein phosphatase mu) domain, and a frizzled domain. See review by Bugge et al. (2009), supra. For example, TMPRSS2 and TMPRSS4, both of which belong to the hepsin/TMPRSS subfamily, have a group A scavenger receptor domain, preceded by a single LDLA domain in the stem region. TMPRSS11D, also known as “HAT” for human airway trypsin-like protease that belongs to the HAT/DESC subfamily, has a single SEA domain. See FIG. 1 of Bugge et al. (2009), supra.

Type II transmembrane serine proteases are produced initially as inactive proenzymes that require activation by cleavage following a basic amino acid residue in a consensus activation motif preceding the protease domain. Some of the activated proteases remain membrane bound as a result of a disulfide bond between the prodomain and the protease domain. The extracellular domains are considered to be critical for cellular localization, activation, inhibition, and/or substrate specificity of these proteases (Bugge et al. (2009), supra; Szabo et al., Int. J. Biochem. Cell Biol. 40: 1297-1316 (2008)).

Various biochemical and pathophysiological information has been documented for members of the type II transmembrane serine proteases. TMPRSS2, TMPRSS4 and TMPRSS11D have been shown in vitro to cleave influenza A hemagglutinin (HA), which is the first essential step in the viral life cycle. Genetically modified rodent animals having a humanized Tmprss gene disclosed herein provide useful in vivo systems that allow for a thorough understanding of the biological functions of the TMPRSS molecules, as well as for screening therapeutic compounds that specifically target human TMPRSS molecules.

Exemplary Tmprss sequences, including mouse, human and humanized Tmprss nucleic acid and protein sequences, are provided in this application and are summarized in the following table. Primer and probe sequences used in the assays described in the examples section, and insertion junction sequences of exemplary humanized Tmprss alleles, are also included in the table.

Summary Description of Sequences

| SEQ ID NO | Description | Features |
|---|---|---|
| 1 | Mus musculus | Length: 3175 bp |
| | Tmprss2, mRNA, | CDS: 231-1703 |
| | NM_015775.2 | Exons: 1-177; 178-245 (second exon, |
| | | and first coding exon); 246-465; |
| | | 466-552; 553-672; 673-799; 800-910; |
| | | 911-954; 955-1123; 1124-1299; 1300-1395; |
| | | 1396-1538; 1539-1691; 1692-3161. |
| 2 | Mus musculus | Length: 490 aa |
| | Tmprss2, protein | |
| 3 | Homo sapiens | Length: 3212 bp |
| | TMPRSS2, | CDS: 135-1613 |
| | transcript variant | Exons: 1-78; 79-149 (second exon, and first |
| | 2, mRNA, | coding exon); 150-372; |
| | NM_005656.3 | 373-459; 460-579; 580-706; 707-817; |
| | | 818-861; 862-1033; 1034-1209; |
| | | 1210-1305; 1306-1448; 1449-1601; |
| | | 1602-3204. |
| 4 | Homo sapiens | Length: 492 aa |
| | TMPRSS2, | Ectodomain: begins at W106. |
| | transcript variant | |
| | 2, protein | |
| 5 | Humanization | Length: 27,947 bp |
| | Tmprss2 | 1-84: mouse sequence |
| | genomic | 85-25175: human sequence (total 25091 bp) |
| | fragment | 25176-27866: XhoI-LoxP-Cassette-loxP- |
| | | ICeUI-NheI (total 2691 bp) |
| | | 27867-27947: mouse sequence |
| 6 | Humanization | Length: 25,333 bp |
| | Tmprss2 | 1-84: mouse sequence |
| | genomic fragment | 85-25175: human sequence (total 25091 bp) |
| | with cassette | 25176-25252: XhoI-loxP-ICeUI-NheI (77 bp) |
| | deleted | 25253-25333: mouse sequence |
| 7 | Humanized | Length: 491 aa |
| | Tmprss2 protein | |
| 8 | Mus musculus | Length: 2267 bp |
| | Tmprss4, | CDS: 289-1596 |
| | mRNA, | Exons: 1-291 (first exon and first coding exon); |
| | NM_145403.2 | 292-325; 326-439; 440-592; 593-722; 723-824; |
| | | 825-865; 866-1025; 1026-1192; 11.93-1291; |
| | | 1292-1434; 1435-1584; 1585-2267. |
| 9 | Mus musculus | Length: 435 aa |
| | Tmprss4, protein | |
| 10 | Homo sapiens | Length: 3543 bp |
| | TMPRSS4, | CDS: 292-1599 |
| | transcript variant | Exons: 1-294 (first exon and first coding exon); |
| | 4, mRNA, | 295-328; 329-442; 443-595; 596-725; 726-827; |
| | NM_001173551.1 | 828-868; 869-1028; 1029-1195; 1196-1294; |
| | | 1295-1437; 1438-1587; 1588-3529. |
| 11 | Homo sapiens | Length: 437 aa |
| | TMPRSS4, | Ectodomain: begins at K54. |
| | transcript variant | |
| | 4, protein | |
| 12 | Humanization | Length: 20,078 bp |
| | Tmprss4 | 1-18: mouse sequence |
| | genomic | 19-5014: SalI/XhoI-LoxP-hUbi-EM7-Neo-Pm- |
| | fragment | Cre-loxP-ICeuI-NheI (total 4996 bp) |
| | containing | 5015-19977: HUMAN sequence (total 14963 |
| | cassette | bp) 19978-20078: mouse sequence |
| 13 | Humanization | Length: 15159 bp |
| | Tmprss4 | 1-18: mouse sequence |
| | genomic | 19-95: SalI/XhoI-LoxP-ICeuI-NheI |
| | fragment | (total 77 bp) 96-15058: |
| | with cassette | HUMAN sequence (total 14963 bp) |
| | deleted | 15059-15159: mouse sequence |
| 14 | Humanized | Length: 435 aa |
| | Tmprss4 | |
| | Protein | |
| 15 | Mus musculus | Length: 2046 bp |
| | Tmprss11d, | CDS: 36-1289 |
| | mRNA, | Exons: 1-43 (first exon and first coding exon), |
| | NM_145561.2 | 44-165, 166-284; 285-352; 353-507; 508-546; |
| | | 547-724; 725-984; 985-1127; 1128-2046. |
| 16 | Mus musculus | Length: 417 aa |
| | Tmprss11d, | |
| | protein | |
| 17 | Homo sapiens | Length: 2800 bp |
| | TMPRSS11D, | CDS: 66-1322 |
| | mRNA, | Exons: 1-73 (first exon and first coding exon); |
| | NM_004262.2 | 74-195; 196-314; 315-382; 383-540; 541-579; |
| | | 580-757; 758-1017; 1018-1160; 1161-2783. |
| 18 | Homo sapiens | Length: 418 aa |
| | TMPRSS11D, | Ectodomain begins at A42. |
| | protein | |
| 19 | Humanization | Length: 38,992 |
| | Tmprss11d | 1-19: mouse sequence |
| | genomic | 20-33,946: HUMAN sequence |
| | fragment | (total 33,927 bp) 33,947-38,942: |
| | containing | XhoI-LoxP-hUbi-EM7-Neo- |
| | cassette | Pm-Cre-loxP-ICeuI-NheI (total 4,996 bp) |
| | | 38,943-38,992: mouse sequence |
| 20 | Humanization | Length: 34,073 bp |
| | Tmprss11d | 1-19: mouse sequence |
| | genomic | 20-33,946: HUMAN sequence |
| | fragment | (total 33,927 bp) 33,947-34,023: |
| | with cassette | XhoI-LoxP-ICeuI-NheI (77 bp) |
| | deleted | 34,024-34,073: mouse sequence |
| 21 | Humanized | 418 aa |
| | Tmprss11d | |
| | Protein | |
| 22 | 5′ mouse/5′ | 5′ mouse//5′ human |
| | human junction | |
| | sequence for | |
| | Tmprss2 | |
| | humanization | |
| 23 | 3′ human/cassette | Human//XhoI//loxP Cassette |
| | junction | |
| | sequence for | |
| | Tmprss2 | |
| | humanization | |
| 24 | Cassette/3′ | Cassette (loxP)/ICEUI//NheI//mouse |
| | mouse junction | |
| | sequence for | |
| | Tmprss2 | |
| | humanization | |
| 25 | 3′ human/loxP/ | 3′ human//XhoI//(loxP)/ICEUI//NheI//3′ |
| | 3′ mouse | mouse |
| | junction for | |
| | Tmprss2 | |
| | humanization | |
| 26-37 | Primers and | Table 1 |
| | probes for loss of | |
| | allele and gain | |
| | of allele assays | |
| | for Tmprss2 | |
| | humanization | |
| 38 | 5′ mouse/Cassette | 5′ mouse//SalI-XhoI//(loxP) Cassette |
| | junction | |
| | sequence | |
| | for Tmprss4 | |
| | humanization | |
| 39 | Cassette/5′ | Cassette (loxP)/ICEUI//NheI//5′ human |
| | human junction | |
| | sequence | |
| | for Tmprss4 | |
| | humanization | |
| 40 | 3′ human/3′ | 3′ human/3′ mouse |
| | mouse junction | |
| | sequence | |
| | for Tmprss4 | |
| | humanization | |
| 41 | 5′ mouse/loxP/ | 5′ mouse//SalI/XhoI//(loxP)/ |
| | 5′ human | ICEUI//NheI//5′ human |
| | junction for | |
| | Tmprss4 | |
| | humanization | |
| 42-56 | Primers and | Table 2 |
| | probes for loss of | |
| | allele and gain | |
| | of allele assays | |
| | for Tmprss4 | |
| | humanization | |
| 57 | 5′ mouse/5′ | 5′ mouse//5′ human |
| | human junction | |
| | sequence for | |
| | Tmprss11d | |
| | humanization | |
| 58 | 3′ human/cassette | 3′ human//XhoI//(loxP) Cassette |
| | junction | |
| | sequence for | |
| | Tmprss11d | |
| | humanization | |
| 59 | Cassette/3′ | Cassette (loxP)/ICUEI//NheI//3′ mouse |
| | mouse junction | |
| | sequence for | |
| | Tmprss11d | |
| | humanization | |
| 60 | 3′ human/ | 3′ human//XhoI//(loxP)/ |
| | loxP/3′ mouse | ICEUI//NheI//3′ mouse |
| | junction for | |
| | Tmprss11d | |
| | humanization | |
| 61-72 | Primers and | Table 3 |
| | probes for loss of | |
| | allele and gain | |
| | of allele assays | |
| | for Tmprss11d | |
| | humanization | |

### Humanized Tmprss Rodent Animals

In one aspect, the present invention provides rodent animals that contain in the germline a humanized Tmprss gene encoding a humanized Tmprss protein.

The term “humanized”, when used in the context of nucleic acids or proteins, refers to nucleic acids or proteins whose structures (i.e., nucleotide or amino acid sequences) include portions that correspond substantially or identically with structures of a particular gene or protein found in nature in a rodent animal, and also include portions that differ from that found in the relevant rodent gene or protein and instead correspond more closely or identically with structures found in a corresponding human gene or protein. A rodent containing a humanized gene or expressing a humanized protein is a “humanized” rodent.

In some embodiments, a rodent of the present invention is selected from a mouse, a rat, and a hamster. In some embodiments, a rodent of the present invention is selected from the superfamily Muroidea. In some embodiments, a genetically modified rodent of the present invention is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some certain embodiments, a genetically modified rodent of the present invention is selected from a true mouse or rat (family Muridae), a gerbil, a spiny mouse, and a crested rat. In some certain embodiments, a genetically modified mouse of the present invention is from a member of the family Muridae.

In some embodiments, the rodent disclosed herein contains a humanized Tmprss gene in the genome that includes a nucleotide sequence of an endogenous rodent Tmprss gene and a nucleotide sequence of a human TMPRSS gene, wherein the nucleotide sequence of the endogenous rodent Tmprss gene and the nucleotide sequence of the human TMPRSS gene are operably linked to each other such that the humanized Tmprss gene encodes a Tmprss protein and is under control of a 5′ regulatory element(s), such as the promoter and/or enhancer(s), of the endogenous rodent Tmprss gene.

The present invention is particularly directed to like-for-like humanization; in other words, a nucleotide sequence of an endogenous rodent Tmprss gene is operably linked to a nucleotide sequence of a cognate human TMPRSS gene to form a humanized gene. For example, in some embodiments, a nucleotide sequence of an endogenous rodent Tmprss2 gene is operably linked to a nucleotide sequence of a human TMPRSS2 gene to form a humanized Tmprss2 gene. In other embodiments, a nucleotide sequence of an endogenous rodent Tmprss4 gene is operably linked to a nucleotide sequence of a human TMPRSS4 gene to form a humanized Tmprss4 gene. In still other embodiments, a nucleotide sequence of an endogenous rodent Tmprss11d gene is operably linked to a nucleotide sequence of a human TMPRSS11D gene to form a humanized Tmprss11d gene.

In some embodiments, a genetically modified rodent of this invention contains a humanized Tmprss gene in its genome, wherein the humanized Tmprss gene encodes a humanized Tmprss protein that contains an ectodomain that is substantially identical with the ectodomain of a human TMPRSS protein. The term “ectodomain” refers to the portion of a transmembrane protein that extends outside of the cell membrane, i.e., the extracellular portion of a transmembrane protein. The ectodomain of a TMPRSS molecule includes a protease domain and a stem region that links the transmembrane domain with the protease domain. By an ectodomain or polypeptide that is “substantially identical with the ectodomain of a human TMPRSS protein”, it is meant in some embodiments, a polypeptide that is at least 85%, 90%, 95%, 95%, 99% or 100% identical in sequence with the ectodomain of a human TMPRSS protein; in some embodiments, a polypeptide that differs from the ectodomain of a human TMPRSS protein by not more than 10, 9, 8, 7, 6, 5, 4, 3, 2 or 1 amino acid(s); in some embodiments, a polypeptide that differs from the ectodomain of a human TMPRSS protein only at the N- or C-terminus of the ectodomain, e.g., by lacking amino acids or having additional amino acids at the at the N- or C-terminus of the ectodomain; and in some embodiments, a polypeptide that is substantially the ectodomain of a human TMPRSS protein. By “substantially the ectodomain” of a human TMPRSS protein, it is meant a polypeptide that is identical with the ectodomain, or differs from the ectodomain by lacking 1-5 (i.e., 1, 2, 3, 4 or 5) amino acids or having additional 1-5 amino acids at the N- or C-terminus.

In some embodiments, the humanized Tmprss gene encodes a humanized Tmprss protein that further contains a cytoplasmic and transmembrane portion that is substantially identical with the cytoplasmic and transmembrane portion of an endogenous rodent Tmprss protein. By a cytoplasmic and transmembrane portion or polypeptide that is “substantially identical with the cytoplasmic and transmembrane portion of an endogenous rodent Tmprss protein”, it is meant in some embodiments, a polypeptide that is at least 85%, 90%, 95%, 95%, 99% or 100% identical in sequence with the cytoplasmic and transmembrane portion of an endogenous rodent Tmprss protein; in some embodiments, a polypeptide that differs from the cytoplasmic and transmembrane portion of an endogenous rodent Tmprss protein by not more than 10, 9, 8, 7, 6, 5, 4, 3, 2 or 1 amino acid(s); in some embodiments, a polypeptide that differs from the cytoplasmic and transmembrane portion of an endogenous rodent Tmprss protein only at the C-terminus, e.g., by lacking amino acids or having additional amino acids at the at the C-terminus of the transmembrane domain; and in some embodiments, a polypeptide composed of the cytoplasmic domain and substantially the transmembrane domain of an endogenous rodent Tmprss protein. By “substantially the transmembrane domain” of an endogenous rodent Tmprss protein, it is meant a polypeptide that is identical with the transmembrane domain, or differs from the transmembrane domain by lacking 1-5 amino acids or having additional 1-5 amino acids at the C-terminus.

In some embodiments, the humanized Tmprss gene in the genome of a genetically modified rodent includes a nucleotide sequence of an endogenous rodent Tmprss gene and a nucleotide sequence of a cognate human TMPRSS gene, wherein the nucleotide sequence of the cognate human TMPRSS gene encodes a polypeptide substantially identical to the ectodomain of the human TMPRSS protein encoded by the human TMPRSS gene. In certain embodiments, the nucleotide sequence of a cognate human TMPRSS gene in a humanized Tmprss gene encodes the ectodomain of the human TMPRSS protein encoded by the human TMPRSS gene.

In some embodiments, the humanized Tmprss gene in the genome of a genetically modified rodent includes a nucleotide sequence of an endogenous rodent Tmprss gene and a nucleotide sequence of a cognate human TMPRSS gene, wherein the nucleotide sequence of an endogenous rodent Tmprss gene encodes a polypeptide substantially identical to the cytoplasmic and transmembrane portion of the endogenous rodent Tmprss protein encoded by the rodent Tmprss gene. In specific embodiments, the nucleotide sequence of an endogenous rodent Tmprss gene present in a humanized Tmprss gene encodes the cytoplasmic and transmembrane domains of the endogenous rodent Tmprss protein encoded by the endogenous rodent Tmprss gene.

In some embodiments, a humanized Tmprss gene results from a replacement of a nucleotide sequence of an endogenous rodent Tmprss gene at an endogenous rodent Tmprss locus with a nucleotide sequence of a cognate human TMPRSS gene.

In some embodiments, a contiguous genomic sequence of a rodent Tmprss gene at an endogenous rodent Tmprss locus has been replaced with a contiguous genomic sequence of a cognate human TMPRSS gene to form a humanized Tmprss gene.

In specific embodiments, a contiguous genomic sequence of a human TMPRSS gene inserted into an endogenous rodent Tmprss gene includes exons, in full or in part, of a human TMPRSS gene, that encode an ectodomain that is substantially identical with the ectodomain of the human TMPRSS protein encoded by the human TMPRSS gene.

In certain embodiments, the genomic sequence of an endogenous rodent Tmprss gene that remains at an endogenous rodent Tmprss locus after the humanization replacement and is operably linked to the inserted contiguous human TMPRSS genomic sequence encodes a cytoplasmic and transmembrane portion that is substantially identical with the cytoplasmic and transmembrane portion of the endogenous rodent Tmprss protein encoded by the endogenous rodent Tmprss gene.

In circumstances where an endogenous Tmprss protein and a human TMPRSS protein share common amino acids near the junction between the transmembrane domain and the ectodomain, it may not be necessary to insert a human TMPRSS genomic sequence that encodes precisely the ectodomain of the human TMPRSS protein. It is possible to insert a slightly longer or shorter genomic sequence of a human TMPRSS gene, which encodes substantially the ectodomain of the human TMPRSS protein, in operable linkage to a genomic sequence of an endogenous rodent Tmprss gene that encodes the cytoplasmic domain and substantially the transmembrane domain of the endogenous rodent Tmprss protein, such that the humanized Tmprss protein encoded by the resulting humanized Tmprss gene includes an ectodomain that is identical with the ectodomain of the human TMPRSS protein and a transmembrane domain that is identical with the transmembrane domain of the endogenous rodent Tmprss protein.

In some embodiments, the nucleotide sequence of a human TMPRSS gene included in a humanized Tmprss gene also includes the 3′ untranslated region (“UTR”) of the human TMPRSS gene. In certain embodiments, in addition to the 3′ UTR of a human TMPRSS gene, a humanized Tmprss gene also includes an additional human genomic sequence from the human TMPRSS gene locus, following the human TMPRSS 3′ UTR. The additional human genomic sequence can consist of at least 10-200 bp, e.g., 50 bp, 75 bp, 100 bp, 125 bp, 150 bp, 175 bp, 200 bp, or more, found in the human TMPRSS gene locus immediately downstream of the 3′ UTR of the human TMPRSS gene. In other embodiments, the nucleotide sequence of a human TMPRSS gene present in a humanized Tmprss gene does not include a human 3′ UTR; instead, the 3′ UTR of an endogenous rodent Tmprss gene is included and follows immediately the stop codon of the humanized Tmprss gene. For example, a humanized Tmprss gene can include a nucleotide sequence of an endogenous rodent Tmprss gene containing exon sequences encoding the cytoplasmic and transmembrane domains of the endogenous rodent Tmprss protein, followed by a nucleotide sequence of a human TMPRSS gene containing exon sequences encoding the ectodomain through the stop codon of the human TMPRSS protein, with the 3′ UTR of the endogenous rodent Tmprss gene following immediately after the stop codon.

In some embodiments, a humanized Tmprss gene results in an expression of the encoded humanized Tmprss protein in a rodent. In some embodiments, a humanized Tmprss protein is expressed in a pattern comparable with, or substantially the same as, a counterpart rodent Tmprss protein in a control rodent (e.g., a rodent without the humanized Tmprss gene). In some embodiments, a humanized Tmprss protein is expressed at a level comparable with, or substantially the same as, a counterpart rodent Tmprss protein in a control rodent (e.g., a rodent without the humanized Tmprss gene). In certain embodiments, a humanized Tmprss protein is expressed and detected at the cell surface. In certain embodiments, a humanized Tmprss protein or a soluble form (e.g., a shed ectodomain form) is expressed and detected in serum of a rodent, e.g., at a level comparable with, or substantially the same as, a counterpart rodent Tmprss protein or a soluble form thereof in a control rodent. In the context of comparing a humanized gene or protein in a humanized rodent with an endogenous rodent gene or protein in a control rodent, the term “comparable” means that the molecules or levels being compared may not be identical to one another but are sufficiently similar to permit comparison there between so that conclusions may reasonably be drawn based on differences or similarities observed; and the term “substantially the same” in referring to expression levels means that the levels being compared are not different from one another by more than 20%, 19%, 18%, 17%, 16%, 1⁵%, 4%1, 3%1, 12%, 11%, 10%, 9%, 8%, 7%, 6%, 5%, 4%, 3%, 2%, or 1%.

In some embodiments, the present invention further provides an isolated cell or tissue from a rodent animal as described herein. In some embodiments, a cell is selected from a dendritic cell, lymphocyte (e.g., a B or T cell), macrophage and a monocyte. In some embodiments, a tissue is selected from adipose, bladder, brain, breast, bone marrow, eye, heart, intestine, kidney, liver, lung, lymph node, muscle, pancreas, plasma, serum, skin, spleen, stomach, thymus, testis, ovum, and a combination thereof.

In some embodiments, the present invention provides a rodent embryonic stem cell whose genome contains a humanized Tmprss gene as described herein. In some embodiments, a rodent embryonic stem cell is a mouse embryonic stem cell. In other embodiments, a rodent embryonic stem cell is a rat embryonic stem cell. A rodent embryonic stem cell containing a humanized Tmprss gene in its genome can be used to make a humanized rodent animal, as further described herein below.

In some embodiments, a rodent provided herein is heterozygous for a humanized Tmprss gene in its genome. In other embodiments, a rodent provided herein is homozygous for a humanized Tmprss gene in its genome.

In certain embodiments, a rodent includes multiple, i.e., two or more, humanized Tmprss genes in its genome. In other words, two or more different endogenous Tmprss loci in a rodent have been humanized using nucleotide sequences of cognate human TMPRSS genes. For example, a rodent has been humanized at two or more of the gene loci selected from: Tmprss2, Tmprss4, and Tmprss11d.

Exemplary humanized Tmprss2 rodents (such as mice), humanized Tmprss4 rodents (such as mice), and humanized Tmprss11d rodents (such as mice) are further described below.

### Humanized Tmprss2 Rodents

In some embodiments, this invention provides a rodent whose genome contains a humanized Tmprss2 gene that includes a nucleotide sequence of an endogenous rodent Tmprss2 gene and a nucleotide sequence of a human TMPRSS2 gene, and that is under control of a 5′ regulatory element(s), such as the promoter and/or enhancer(s), of the endogenous rodent Tmprss2 gene. Examples of rodents include mice and rats.

In some embodiments, a humanized Tmprss2 gene encodes a humanized Tmprss2 protein that contains an ectodomain that is substantially identical with the ectodomain of a human TMPRSS2 protein.

In specific embodiments, the human TMPRSS2 protein has an amino acid sequence having at least 85%, 90%, 95%, 98%, 99% or 100% identity with the amino acid sequence as set forth in SEQ ID NO: 4.

In some embodiments, a humanized Tmprss2 protein contains the C-terminal 387 amino acids of a human TMPRSS2 protein, for example, amino acids 106 to 492 of a human TMPRSS2 protein. In some embodiments, a humanized Tmprss2 protein contains an ectodomain that is substantially identical with the amino acid sequence composed of W106 to G492 of SEQ ID NO: 4. In specific embodiments, a humanized Tmprss2 protein contains an ectodomain having at least 85%, 90%, 95%, 98%, 99% or 100% identity with the amino acid sequence composed of W106 to G492 of SEQ ID NO: 4; an ectodomain that differs from the amino acid sequence composed of W106 to G492 of SEQ ID NO: 4 by not more than 10, 9, 8, 7, 6, 5, 4, 3, 2 or 1 amino acid(s); or an ectodomain that differs from the amino acid sequence composed of W106 to G492 of SEQ ID NO: 4 only at the N- or C-terminus of the ectodomain, e.g., lacking 1-5 amino acids or having additional 1-5 amino acids at the at the N- or C-terminus.

In some embodiments, a humanized Tmprss2 protein further contains a cytoplasmic and transmembrane portion that is substantially identical with the cytoplasmic and transmembrane portion of an endogenous rodent Tmprss2 protein. In some embodiments, a humanized Tmprss2 protein further includes the transmembrane domain and the cytoplasmic domain of an endogenous rodent Tmprss2 protein.

In specific embodiments, a humanized Tmprss2 protein contains the transmembrane domain and the cytoplasmic domain of an endogenous rodent Tmprss2 protein, and the ectodomain of a human TMPRSS2 protein. In particular embodiments, a humanized Tmprss2 gene encodes a humanized Tmprss2 protein having the amino acid sequence as set forth in SEQ ID NO: 7.

In some embodiments, a humanized Tmprss2 gene results from a replacement of a nucleotide sequence of an endogenous rodent Tmprss2 gene at an endogenous rodent Tmprss2 locus with a nucleotide sequence of a human TMPRSS2 gene.

In some embodiments, a contiguous genomic sequence of an endogenous rodent Tmprss2 gene at an endogenous rodent Tmprss2 locus has been replaced with a contiguous genomic sequence of a human TMPRSS2 gene to form a humanized Tmprss2 gene.

In specific embodiments, the contiguous genomic sequence of a human TMPRSS2 gene inserted into an endogenous rodent Tmprss2 gene includes exon sequences, i.e., exons in full or in part, of a human TMPRSS2 gene, that encode an ectodomain that is substantially identical to the ectodomain of the human TMPRSS2 protein encoded by the human TMPRSS2 gene. In circumstances where an endogenous Tmprss2 protein and a human TMPRSS2 protein share common amino acids near the junction of the transmembrane domain and the ectodomain, it may not be necessary to insert a human TMPRSS2 genomic sequence that encodes precisely the ectodomain of the human TMPRSS2 protein, and it is possible to use a slightly longer or shorter human TMPRSS2 genomic sequence that encodes substantially the ectodomain of a human TMPRSS2 protein in order to make a humanized Tmprss2 protein having an ectodomain that is identical with the ectodomain of the human TMPRSS2 protein.

In specific embodiments, a contiguous genomic sequence of a human TMPRSS2 gene being inserted into an endogenous rodent Tmprss2 gene contains at least coding exon 4 through the stop codon in coding exon 13 of the human TMPRSS2 gene.

In certain embodiments, a contiguous genomic sequence of a human TMPRSS2 gene being inserted into an endogenous rodent Tmprss2 gene contains intron 3 and coding exon 4 through the stop codon in coding exon 13 of the human TMPRSS2 gene. In particular embodiments, a contiguous genomic sequence of a human TMPRSS2 gene being inserted into an endogenous rodent Tmprss 2 gene contains a 3′ portion of coding exon 3, intron 3, and coding exon 4 through the stop codon in coding exon 13 of the human TMPRSS2 gene. In specific embodiments, the 3′ portion of coding exon 3 of a human TMPRSS2 gene included in the humanization is about 5-10 base pair in length, i.e., about 5, 6, 7, 8, 9 or 10 base pair of the 3′ end of coding exon 3.

In some embodiments, a contiguous genomic sequence of a human TMPRSS2 gene being inserted into an endogenous rodent Tmprss2 gene also contains the 3′ UTR of the human TMPRSS2 gene. In specific embodiments, the entire coding exon 13 of a human TMPRSS2 gene is included in the contiguous human TMPRSS2 genomic sequence for humanization, which includes the 3′ UTR of the human TMPRSS2 gene. In particular embodiments, a contiguous genomic sequence of a human TMPRSS2 gene includes an additional human genomic sequence downstream of the 3′ UTR of the human TMPRSS2 gene. The additional human genomic sequence can be a sequence of at least 10-200 bp, or at least 10, 20, 30, 40, 50, 75, 100, 125, 150, 175, or 200 bp, that is found immediately downstream of the 3′ UTR of the human TMPRSS2 gene at a human TMPRSS2 locus.

In some embodiments, the nucleotide sequence of an endogenous rodent Tmprss2 gene remaining at a humanized Tmprss2 locus encodes a polypeptide that is substantially identical with the cytoplasmic and transmembrane portion of the endogenous rodent Tmprss2 protein. In circumstances where an endogenous Tmprss2 protein and a human TMPRSS2 protein share common amino acids near the junction of the transmembrane domain and the ectodomain, it may not be necessary to maintain the endogenous rodent Tmprss2 genomic sequence that encodes precisely the transmembrane domain of the endogenous rodent Tmprss2 protein, and it is possible to maintain a slightly longer or shorter rodent Tmprss2 genomic sequence that encodes substantially the transmembrane domain of the endogenous rodent Tmprss2 protein in the humanization replacement in order to encode a humanized Tmprss2 protein having a transmembrane domain that is identical with the transmembrane of the endogenous rodent Tmprss2 protein. In some embodiments, the nucleotide sequence of an endogenous rodent Tmprss2 gene remaining at a humanized Tmprss2 locus includes exons 1-2 and a 5′ portion of coding exon 3 of an endogenous rodent Tmprss2 gene, wherein the 5′ portion of coding exon 3 is a substantial portion of codon exon 3, e.g., the entire coding exon 3 except 5-10 base pairs at the 3′ end of coding exon 3.

In specific embodiments, a humanized Tmprss2 gene contains coding exons 1-2 and a 5′ portion of coding exon 3 of an endogenous rodent Tmprss2 gene, and a 3′ portion of coding exon 3 and coding exon 4 through coding exon 13 of a human TMPRSS2 gene, wherein the humanized Tmprss2 gene encodes a humanized Tmprss2 protein that contains a cytoplasmic and transmembrane portion that is substantially identical with the cytoplasmic and transmembrane portion of the rodent Tmprss2 protein, and an ectodomain that is substantially identical with the ectodomain of the human TMPRSS2 protein. In certain embodiments, the humanized Tmprss2 gene encodes a humanized Tmprss2 protein that contains the cytoplasmic domain and the transmembrane domain of the rodent Tmprss2 protein encoded by an endogenous rodent Tmprss2 gene, and the ectodomain of the human TMPRSS2 protein encoded by a human TMPRSS2 gene. In particular embodiments, a humanized Tmprss2 gene encodes a humanized Tmprss2 protein having the amino acid sequence as set forth in SEQ ID NO: 7.

In some embodiments, the exons and introns of a human TMPRSS2 gene and a rodent Tmprss2 gene used in the humanization are those found in SEQ ID NOS: 1, 3 and 5-6.

In some embodiments, a humanized Tmprss2 gene results in an expression of the encoded humanized Tmprss2 protein in a rodent. In some embodiments, a humanized Tmprss2 protein is expressed in a pattern comparable with, or substantially the same as, a counterpart rodent Tmprss2 protein in a control rodent (e.g., a rodent without the humanized Tmprss2 gene). In some embodiments, a humanized Tmprss2 protein is expressed at a level comparable with, or substantially the same as, a counterpart rodent Tmprss2 protein in a control rodent (e.g., a rodent without the humanized Tmprss2 gene). In certain embodiments, a humanized Tmprss2 protein is expressed and detected at the cell surface. In certain embodiments, a humanized Tmprss2 protein or a soluble form (e.g., a shed ectodomain form) is expressed and detected in serum of a rodent, e.g., at a level comparable with, or substantially the same as, a counterpart rodent Tmprss2 protein or a soluble form thereof in a control rodent.

### Humanized Tmprss4 Rodents

In some embodiments, this invention provides a rodent whose genome contains a humanized Tmprss4 gene that includes a nucleotide sequence of an endogenous rodent Tmprss4 gene and a nucleotide sequence of a human TMPRSS4 gene, and that is under control of a 5′ regulatory element(s), such as the promoter and/or an enhancer(s), of the endogenous rodent Tmprss4 gene. Examples of rodents include mice and rats.

In some embodiments, a humanized Tmprss4 gene encodes a humanized Tmprss4 protein that contains an ectodomain that is substantially identical with the ectodomain of a human TMPRSS4 protein. In specific embodiments, the human TMPRSS4 protein has an amino acid sequence having at least 85%, 90%, 95%, 98%, 99% or 100% identity with the amino acid sequence as set forth in SEQ ID NO: 11.

In some embodiments, a humanized Tmprss4 protein contains the C-terminal 384 amino acids of a human TMPRSS4 protein, for example, amino acids 54 to 437 of a human TMPRSS4 protein. In some embodiments, a humanized Tmprss4 protein contains an ectodomain that is substantially identical with the amino acid sequence composed of K54 to L437 of SEQ ID NO: 11. In specific embodiments, a humanized Tmprss4 protein contains an ectodomain having at least 85%, 90%, 95%, 98%, 99% or 100% identity with the amino acid sequence composed of K54 to L437 of SEQ ID NO: 11; an ectodomain that differs from the amino acid sequence composed of K54 to L437 of SEQ ID NO: 11 by not more than 10, 9, 8, 7, 6, 5, 4, 3, 2 or 1 amino acid(s); or an ectodomain that differs from the amino acid sequence composed of K54 to L437 of SEQ ID NO: 11 only at the N- or C-terminus of the ectodomain, e.g., lacking 1-5 amino acids or having additional 1-5 amino acids at the N- or C-terminus.

In some embodiments, a humanized Tmprss4 protein further contains a cytoplasmic and transmembrane portion that is substantially identical with the cytoplasmic and transmembrane portion of an endogenous rodent Tmprss4 protein. In some embodiments, a humanized Tmprss4 protein further includes the transmembrane domain and the cytoplasmic domain of an endogenous rodent Tmprss4 protein.

In specific embodiments, a humanized Tmprss4 protein contains the transmembrane domain and the cytoplasmic domain of an endogenous rodent Tmprss4 protein, and the ectodomain of a human TMPRSS4 protein. In particular embodiments, a humanized Tmprss4 gene encodes a humanized Tmprss4 protein having the amino acid sequence as set forth in SEQ ID NO: 14.

In some embodiments, a humanized Tmprss4 gene results from a replacement of a nucleotide sequence of an endogenous rodent Tmprss4 gene at an endogenous rodent Tmprss4 locus with a nucleotide sequence of a human TMPRSS4 gene.

In some embodiments, a contiguous genomic sequence of an endogenous rodent Tmprss4 gene at an endogenous rodent Tmprss4 locus has been replaced with a contiguous genomic sequence of a human TMPRSS4 gene to form a humanized Tmprss4 gene.

In specific embodiments, the contiguous genomic sequence of a human TMPRSS4 gene inserted into an endogenous rodent Tmprss4 gene includes exon sequences, i.e., exons in full or in part, of a human TMPRSS4 gene that encode an ectodomain that is substantially identical with the ectodomain of the human TMPRSS4 protein encoded by the human TMPRSS4 gene. In circumstances where an endogenous Tmprss4 protein and a human TMPRSS4 protein share common amino acids near the junction of the transmembrane domain and the ectodomain, it may not be necessary to insert a human TMPRSS4 genomic sequence that encodes precisely the ectodomain of the human TMPRSS4 protein, and it is possible to use a slightly longer or shorter human TMPRSS4 genomic sequence that encodes substantially the ectodomain of a human TMPRSS4 protein in order to make a humanized Tmprss4 protein having an ectodomain that is identical with the ectodomain of the human TMPRSS4 protein.

In specific embodiments, a contiguous genomic sequence of a human TMPRSS4 gene being inserted into an endogenous rodent Tmprss4 gene contains at least coding exon 4 through the stop codon in coding exon 13 of the human TMPRSS4 gene.

In certain embodiments, a contiguous genomic sequence of a human TMPRSS4 gene being inserted into an endogenous rodent Tmprss4 gene includes a 3′ portion of intron 3, and coding exon 4 through the stop codon in coding exon 13 of a human TMPRSS4 gene. In specific embodiments, the 3′ portion of intron 3 of a human TMPRSS4 gene included in the humanization is about 140-160 base pair in length, i.e., about 140, 145, 150, 155, 160 base pair of the 3′ end of intron 3.

In some embodiments, a contiguous genomic sequence of a human TMPRSS4 gene being inserted into an endogenous rodent Tmprss4 gene contains the 3′ UTR of the human TMPRSS4 gene. In specific embodiments, a contiguous genomic sequence of a human TMPRSS4 gene being inserted into an endogenous rodent Tmprss4 gene does not contain the 3′ UTR of the human TMPRSS4 gene, and the 3′ UTR of the endogenous rodent Tmprss4 gene follows immediately after the stop codon in the humanized Tmprss4 gene.

In some embodiments, the nucleotide sequence of an endogenous rodent Tmprss4 gene remaining at a humanized Tmprss4 locus encodes a polypeptide that is substantially identical with the cytoplasmic and transmembrane portion of the endogenous rodent Tmprss4 protein. In circumstances where an endogenous Tmprss4 protein and a human TMPRSS4 protein share common amino acids near the junction of the transmembrane domain and the ectodomain, it may not be necessary to maintain the endogenous rodent Tmprss4 genomic sequence that encodes precisely the transmembrane domain of the endogenous rodent Tmprss4 protein, and it is possible to maintain a slightly longer or shorter rodent Tmprss4 genomic sequence that encodes substantially the transmembrane domain of the endogenous rodent Tmprss4 protein in the humanization replacement in order to encode a humanized Tmprss4 protein having a transmembrane domain that is identical with the transmembrane of the endogenous rodent Tmprss4 protein.

In specific embodiments, a humanized Tmprss4 gene contains coding exons 1-3 of an endogenous rodent Tmprss4 gene, and coding exon 4 through the stop codon of coding exon 13 of a human TMPRSS4 gene. In particular embodiments, a humanized Tmprss4 gene contains coding exons 1-3 and a 5′ portion of intron 3 of an endogenous rodent Tmprss4 gene, and a 3′ portion of intron 3 and coding exon 4 through the stop codon of coding exon 13 of a human TMPRSS4 gene. In certain embodiments, the humanized Tmprss4 gene encodes a humanized Tmprss4 protein that contains the cytoplasmic domain and the transmembrane domain of the rodent Tmprss4 protein encoded by an endogenous rodent Tmprss4 gene, and the ectodomain of the human TMPRSS4 protein encoded by a human TMPRSS4 gene. In particular embodiments, a humanized Tmprss4 gene encodes a humanized Tmprss4 protein having the amino acid sequence as set forth in SEQ ID NO: 14.

In some embodiments, the exons and introns of a human TMPRSS4 gene and a rodent Tmprss4 gene used in the humanization are those found in SEQ ID NOS: 8, 10 and 12-13.

In some embodiments, a humanized Tmprss4 gene results in an expression of the encoded humanized Tmprss4 protein in a rodent. In some embodiments, a humanized Tmprss4 protein is expressed in a pattern comparable with, or substantially the same as, a counterpart rodent Tmprss4 protein in a control rodent (e.g., a rodent without the humanized Tmprss4 gene encoding the humanized Tmprss4 protein). In some embodiments, a humanized Tmprss4 protein is expressed at a level comparable with, or substantially the same as, a counterpart rodent Tmprss4 protein in a control rodent (e.g., a rodent without the humanized Tmprss4 gene encoding the humanized Tmprss4 protein). In certain embodiments, a humanized Tmprss4 protein is expressed and detected at the cell surface. In certain embodiments, a humanized Tmprss4 protein or a soluble form (e.g., a shed ectodomain form) is expressed and detected in serum of a rodent, e.g., at a level comparable with, or substantially the same as, a counterpart rodent Tmprss4 protein or a soluble form thereof in a control rodent.

### Humanized Tmprss11d Rodents

In some embodiments, this invention provides a rodent whose genome contains a humanized Tmprss11d gene that includes a nucleotide sequence of an endogenous rodent Tmprss11d gene and a nucleotide sequence of a human TMPRSS11D gene, and that is under control of a 5′ regulatory element(s), such as the promoter and/or enhancer(s) of the endogenous rodent Tmprss11d gene. Examples of rodents include mice and rats.

In some embodiments, a humanized Tmprss11d gene encodes a humanized Tmprss11d protein that contains an ectodomain that is substantially identical with the ectodomain of a human TMPRSS11D protein.

In specific embodiments, the human TMPRSS1ID protein has an amino acid sequence having at least 85%, 90%, 95%, 98%, 99% or 100% identity with the amino acid sequence as set forth in SEQ ID NO: 18.

In some embodiments, a humanized Tmprss11d protein contains the C-terminal 377 amino acids of a human TMPRSS11D protein, for example, amino acids 42 to 418 of a human TMPRSS11D protein. In some embodiments, a humanized Tmprss11d protein contains an ectodomain that is substantially identical with the amino acid sequence composed of A42 to 1418 of SEQ ID NO: 18. In specific embodiments, a humanized Tmprss11d protein contains an ectodomain having at least 85%, 90%, 95%, 98%, 99% or 100% identity with the amino acid sequence composed of A42 to 1418 of SEQ ID NO: 18; an ectodomain that differs from the amino acid sequence composed of A42 to 1418 of SEQ ID NO: 18 by not more than 10, 9, 8, 7, 6, 5, 4, 3, 2 or 1 amino acid(s); or an ectodomain that differs from the amino acid sequence composed of A42 to 1418 of SEQ ID NO: 18 only at the N- or C-terminus, e.g., by lacking 1-5 amino acids or having additional 1-5 amino acids at the N- or C-terminus.

In some embodiments, a humanized Tmprss11d protein further contains a cytoplasmic and transmembrane portion that is substantially identical with the cytoplasmic and transmembrane portion of an endogenous rodent Tmprss11d protein. In some embodiments, a humanized Tmprss11d protein includes the transmembrane domain and the cytoplasmic domain of an endogenous rodent Tmprss11d protein.

In specific embodiments, a humanized Tmprss11d protein contains the transmembrane domain and the cytoplasmic domain of an endogenous rodent Tmprss11d protein, and the ectodomain of a human TMPRSS11D protein. In particular embodiments, a humanized Tmprss11d gene encodes a humanized Tmprss11d protein having the amino acid sequence as set forth in SEQ ID NO: 21.

In some embodiments, a humanized Tmprss11d gene results from a replacement of a nucleotide sequence of an endogenous rodent Tmprss11d gene at an endogenous rodent Tmprss11d locus with a nucleotide sequence of a human TMPRSS11D gene.

In some embodiments, a contiguous genomic sequence of an endogenous rodent Tmprss11d gene at an endogenous rodent Tmprss11d locus has been replaced with a contiguous genomic sequence of a human TMPRSS11D gene to form a humanized Tmprss11d gene. In specific embodiments, the contiguous genomic sequence of a human TMPRSS11D gene inserted into an endogenous rodent Tmprss11d gene includes exon sequences, i.e., exons in full or in part, of a human TMPRSS11D gene that encode an ectodomain that is substantially identical with the ectodomain of the human TMPRSS1ID protein encoded by the human TMPRSS11D gene. In circumstances where an endogenous Tmprss11d protein and a human TMPRSS11D protein share common amino acids near the junction of the transmembrane domain and the ectodomain, it may not be necessary to insert a human TMPRSS11D genomic sequence that encodes precisely the ectodomain of the human TMPRSS11D protein, and it is possible to use a slightly longer or shorter human TMPRSS11D genomic sequence that encodes substantially the ectodomain of a human TMPRSS11D protein in order to make a humanized Tmprss11d protein having an ectodomain that is identical with the ectodomain of the human TMPRSS11D protein.

In specific embodiments, a contiguous genomic sequence of a human TMPRSS11D gene being inserted into an endogenous rodent Tmprss11d gene contains at least coding exon 3 through the stop codon in coding exon 10 of a human TMPRSS11D gene.

In certain embodiments, a contiguous genomic sequence of a human TMPRSS11D gene being inserted into an endogenous rodent Tmprss11d gene contains at least a 3′ portion of intron 2 and coding exon 3 through the stop codon in coding exon 10 of the human TMPRSS11D gene. In specific embodiments, the 3′ portion of intron 2 of a human TMPRSS2 gene included in the humanization is about 444 base pairs in length.

In some embodiments, a contiguous genomic sequence of a human TMPRSS11D gene being inserted into an endogenous rodent Tmprss11d gene contains the 3′ UTR of the human TMPRSS11D gene. In specific embodiments, the entire coding exon 10 of a human TMPRSS11D gene is included in the contiguous human TMPRSS11D genomic sequence for humanization, which includes the 3′ UTR of a human TMPRSS11D gene. In particular embodiments, a contiguous genomic sequence of a human TMPRSS11D gene includes an additional human genomic sequence downstream of the 3′ UTR of the human TMPRSS11D gene. The additional human genomic sequence can be a sequence of 10-200 bp, 50-200 bp, or about 150, 160, 170, 180 bp, that is found immediately downstream of the 3′ UTR of the human TMPRSS11D gene at a human TMPRSS11D locus.

In some embodiments, the nucleotide sequence of an endogenous rodent Tmprss11d gene remaining at a humanized Tmprss11d locus encodes a polypeptide that is substantially identical with the cytoplasmic and transmembrane portion of the endogenous rodent Tmprss11d protein encoded by the endogenous rodent Tmprss11d gene. In circumstances where an endogenous Tmprss11d protein and a human TMPRSS11D protein share common amino acids near the junction of the transmembrane domain and the ectodomain, it may not be necessary to maintain the endogenous rodent Tmprss11d genomic sequence that encodes precisely the transmembrane domain of the endogenous rodent Tmprss11d protein, and it is possible to maintain a slightly longer or shorter rodent Tmprss11d genomic sequence that encodes substantially the transmembrane domain of the endogenous rodent Tmprss11d protein in the humanization replacement in order to encode a humanized Tmprss11d protein having a transmembrane domain that is identical with the transmembrane of the endogenous rodent Tmprss 11d protein.

In specific embodiments, a humanized Tmprss11d gene contains coding exons 1-2 of an endogenous rodent Tmprss11d gene, and coding exon 3 through coding exon 10 of a human TMPRSS11D gene. In certain embodiments, the humanized Tmprss11d gene encodes a humanized Tmprss11d protein that contains the cytoplasmic domain and the transmembrane domain of the rodent Tmprss11d protein encoded by an endogenous rodent Tmprss11d gene, and the ectodomain of the human TMPRSS 11D protein encoded by a human TMPRSS11D gene. In particular embodiments, a humanized Tmprss11d gene encodes a humanized Tmprss11d protein having the amino acid sequence as set forth in SEQ ID NO: 21.

In some embodiments, the exons and introns of a human TMPRSS11D gene and a rodent Tmprss11d gene used in the humanization are those found in SEQ ID NOS: 15, 17 and 19-20.

In some embodiments, a humanized Tmprss11D gene results in an expression of the encoded humanized Tmprss11d protein in a rodent. In some embodiments, a humanized Tmprss11d protein is expressed in a pattern comparable with, or substantially the same as, a counterpart rodent Tmprss11d protein in a control rodent (e.g., a rodent without the humanized Tmprss11d gene encoding the humanized Tmprss11d protein). In some embodiments, a humanized Tmprss11d protein is expressed at a level comparable with, or substantially the same as, a counterpart rodent Tmprss11d protein in a control rodent (e.g., a rodent without the humanized Tmprss11d gene encoding the humanized Tmprss11d protein). In certain embodiments, a humanized Tmprss11d protein is expressed and detected at the cell surface. In certain embodiments, a humanized Tmprss11d protein or a soluble form (e.g., a shed ectodomain form) is expressed and detected in serum of a rodent, e.g., at a level comparable with, or substantially the same as, a counterpart rodent Tmprss11d protein or a soluble form thereof in a control rodent.

### Methods of Making Humanized Tmprss Rodent Animals

Further aspects of this disclosure are directed to methods for making a humanized Tmprss rodent described above, as well as nucleic acid vectors and non-human embryonic stem cells suitable for use in making a humanized Tmprss rodent.

The rodents provided herein can be made using methods known in the art. In exemplary embodiments, a bacterial artificial chromosome (BAC) clone carrying a rodent Tmprss gene can be modified using bacterial homologous recombination and VELOCIGENE® technology (see, e.g., U.S. Pat. No. 6,586,251 and Valenzuela et al. (2003), High-throughput engineering of the mouse genome coupled with high-resolution expression analysis, Nature Biotech. 21(6):652-659). As a result, a rodent Tmprss nucleotide sequence has been deleted from the original BAC clone, and a human Tmprss nucleotide sequence has been inserted, resulting in a modified BAC clone carrying a humanized Tmprss gene, flanked with 5′ and 3′ rodent homology arms. The modified BAC clone, once linearized, can be introduced into rodent embryonic stem (ES) by, e.g., electroporation. Both mouse ES cells and rat ES cells have been described in the art. See, e.g., U.S. Pat. Nos. 7,576,259, 7,659,442, 7,294,754, and US 2008-0078000 A1 (all of which are incorporated herein by reference) describe mouse ES cells and the VELOCIMOUSE® method for making a genetically modified mouse; US 2014/0235933 A1, US 2014/0310828 A1, Tong et al. (2010) Nature 467:211-215, and Tong et al. (2011) NatProtoc. 6(6): doi:10.1038/nprot.2011.338 (all of which are incorporated herein by reference) describe rat ES cells and methods for making a genetically modified rat.

ES cells having a humanized Tmprss gene integrated in the genome can be selected. In some embodiments, ES cells having a humanized Tmprss integrated into an endogenous rodent Tmprss locus can be selected based on loss of rodent allele and/or gain of human allele assays. Selected ES cells are then used as donor ES cells for injection into a pre-morula stage embryo (e.g., 8-cell stage embryo) by using the VELOCIMOUSE® method (see, e.g., U.S. Pat. Nos. 7,576,259, 7,659,442, 7,294,754, and US 2008-0078000 A1), or methods described in US 2014/0235933 A1 and US 2014/0310828 A1. The embryo comprising the donor ES cells is incubated until blastocyst stage and then implanted into a surrogate mother to produce an F0 rodent fully derived from the donor ES cells. Rodent pups bearing the humanized Tmprss gene can be identified by genotyping of DNA isolated from tail snips using loss of rodent allele and/or gain of human allele assays.

Rodents heterozygous for a humanized Tmprss gene can be crossed to generated homozygous rodents. Rodents containing one humanized Tmprss gene can be crossed with rodents containing another humanized Tmprss gene to make rodents containing multiple humanized Tmprss genes. For example, rodents containing a humanized Tmprss2 gene can be crossed with rodents containing a humanized Tmprss4 gene to make rodents containing a humanized Tmprss2 gene and a humanized Tmprss4 gene.

### Methods Employing Rodents Having Humanized Tmprss Genes

Rodents disclosed herein provide a useful in vivo system and source of biological materials (e.g., cells) expressing humanized Tmprss proteins for identifying and testing compounds that specifically target human TMPRSS proteins.

In one aspect, a rodent disclosed herein is used to determine the ability of a candidate compound, such as an inhibitor of a human TMPRSS protein, to treat and/or prevent influenza virus infection.

In some embodiments, a rodent containing a humanized Tmprss gene and expressing a humanized Tmprss protein disclosed herein is administered with a candidate compound prior to experimental influenza virus infection. The prophylactic efficacy of the compound can be evaluated by determining whether the rodent exhibits fewer and/or less severe symptoms of influenza virus infection, and/or improved viability, as compared to control rodent(s).

In other embodiments, a rodent containing a humanized Tmprss gene and expressing a humanized Tmprss protein comprising the ectodomain of a human TMPRSS protein is administered with a candidate inhibitor of that human TMPRSS protein after experimental influenza virus infection. The treatment efficacy of the candidate inhibitor can be evaluated by determining whether the rodent exhibits fewer and/or less severe symptoms of influenza virus infection, and/or improved viability, as compared to control rodent(s).

Suitable control rodents include, e.g., rodents containing a humanized Tmprss gene without being subjected to the experimental infection; and rodents containing a humanized Tmprss gene subjected to the experimental infection without any compound; and rodents containing a humanized Tmprss gene subjected to the experimental infection and a compound known to be therapeutically effective.

Compounds that can be evaluated in the methods of this invention include candidate TMPRSS inhibitors, for example, a small molecule protease inhibitor, a nucleic acid-based inhibitor (e.g., siRNA, ribozyme, antisense construct, etc.), antigen-binding protein (e.g., antibody or antigen-binding fragment thereof), or a blocking peptide/peptide inhibitor. A TMPRSS inhibitor may function by inhibiting or reducing the ability of a TMPRSS protein to proteolytically cleave hemagglutinin precursor protein (HA0) into the HA1 and HA2 subunits.

In some embodiments, a candidate inhibitor is an antibody or antigen-binding fragment thereof. Both monoclonal and polyclonal antibodies are suitable for purposes of this invention. In specific embodiments, the antibody specifically binds to a TMPRSS protein and inhibits the protease activity of that TMPRSS protein and does not substantially inhibit the protease activity of another TMPRSS protein. For example, an anti-TMPRSS2 antibody inhibitor specifically binds to a TMPRSS2 protein and inhibits the protease activity of the TMPRSS2 protein, and has no effect on the proteolytic activity of TMPRSS4 or TMPRSS11D, or reduces the proteolytic activity of TMPRSS4 or TMPRSS11D by no more than 25% (e.g., by 20%, 15%, 10%, 5%, or less) relative to a non-inhibitory control molecule tested under identical or substantially identical experimental conditions.

In some embodiments, the inhibitor is an anti-TMPRSS2 antibody or antigen-binding fragment thereof. In some embodiments, the inhibitor is an anti-TMPRSS4 antibody or antigen-binding fragment thereof. In other embodiments, the inhibitor is an anti-TMPRSS 11D antibody or antigen-binding fragment thereof.

Experimental influenza virus infection can be induced and monitored following known protocols. See, e.g., US 2013/0273070 A1. For example, rodent animals can be administered intranasally with influenza virus. The infected animals can be evaluated to determine the symptoms and severity of the infection. For example, the animals can be analyzed for (1) weight change and survival, (2) cellular changes via flow cytometry, (3) immunochemistry, PAS and H&E staining of whole lungs, and (4) cytokine levels in serum. Control animals known to be susceptible to the virus exhibit a significant increase in the frequency of dendritic cells, the levels influenza-positive alveolar macrophages, neutrophils or epithelial cells in the lungs, and the levels of IFNγ, as compared to uninfected animals.

### EXAMPLES

The following examples are provided so as to describe to those of ordinary skill in the art how to make and use methods and compositions of the invention, and are not intended to limit the scope of what the inventors regard as their invention. Unless indicated otherwise, temperature is indicated in Celsius, and pressure is at or near atmospheric.

### Example 1. Humanization of an Endogenous Tmprss2 Gene

This example illustrates exemplary methods of humanizing an endogenous gene encoding Tmprss2 in a rodent (e.g., a mouse). The methods described in this example can be employed to humanize an endogenous Tmprss2 gene of a rodent using any human sequence, or combination of human sequences (or sequence fragments) as desired.

A targeting vector for humanization of an endogenous Tmprss2 gene was constructed using bacterial artificial chromosome (BAC) clones and VELOCIGENE® technology (see, e.g., U.S. Pat. No. 6,586,251 and Valenzuela et al. (2003) High-throughput engineering of the mouse genome coupled with high-resolution expression analysis, Nature Biotech. 21(6):652-659; incorporated herein by reference).

Briefly, mouse bacterial artificial chromosome (BAC) clone bMQ-264A15 containing a mouse Tmprss2 gene was used and modified as follows. A DNA fragment was generated to include a 5′ mouse homology nucleotide sequence, a human TMPRSS2 genomic DNA of about 25,091 bp (containing the last 7 bp of coding exon 3, intron 3, and coding exon 4 through coding exon 13 (including the 3′ UTR which is part of coding exon 13), of a human TMPRSS2 gene), a self-deleting neomycin cassette of about 2,691 bp, and a 3′ mouse homology sequence. This DNA fragment was used to modify BAC clone bMQ-264A15 through homologous recombination in bacterial cells. As a result, an ectodomain-encoding mouse Tmprss2 genomic fragment (of about 25,291 bp) in the BAC clone was replaced with the human TMPRSS2 genomic fragment of about 25,091 bp, followed by a self-deleting neomycin cassette of about 2691 bp. Specifically, the mouse Tmprss2 genomic fragment that was replaced included the last 7 bp of coding exon 3, intron 3, and coding exon 4 through the stop codon in coding exon 13 of the mouse Tmprss2 gene (FIGS. 1A-1B). The human TMPRSS2 genomic fragment that was inserted included the last 7 bp of coding exon 3, intron 3, and coding exon 4 through coding exon 13 of a human TMPRSS2 gene (including the 3′ UTR of human TMPRSS2), and a human 3′ genomic sequence of 131 bp downstream of the 3′ UTR of human TMPRSS2 (FIGS. 1A-1B). The resulting modified BAC clone included, from 5′ to 3′, (i) a 5′ mouse homology arm containing about 12 kb of mouse genomic DNA including a mouse Tmprss2 5′ UTR, mouse Tmprss2 exon 1 (non-coding), coding exons 1-3 (except the last 7 bp of coding exon 3); (ii) a human TMPRSS2 genomic fragment of about 25,091 bp including the last 7 bp of human coding exon 3, intron 3, human coding exons 4 through 13 (including the 3′ UTR of human TMPRSS2), and a human 3′ genomic sequence; (iii) a self-deleting neomycin cassette of about 2691 bp, followed by (iv) a 3′ mouse homology arm of 45 kb containing the mouse Tmprss2 3′UTR and the remaining mouse genomic DNA in the original BAC clone. See FIGS. 1A-1B.

The junction sequences are also set forth at the bottom of FIG. 1B. The part of the modified BAC clone containing the human TMPRSS2 genomic fragment and the neomycin cassette, as well as the upstream and downstream insertion junctions, is set forth in SEQ ID NO: 5. The amino acid sequence of the protein encoded by the humanized Tmprss2 gene is set forth in SEQ ID NO: 7. An alignment of this humanized Tmprss2 protein (“7010 mutant protein”), a mouse Tmprss2 protein (SEQ ID NO: 2), and a human TMPRSS2 protein (SEQ ID NO: 4), is provided in FIG. 1D.

The modified BAC clone containing the humanized Tmprss2 gene, as described above, was used to electroporate mouse embryonic stem (ES) cells to create modified ES cells comprising a humanized Tmprss2 gene. Positively targeted ES cells containing a humanized Tmprss2 gene were identified by an assay (Valenzuela et al., supra) that detected the presence of the human TMPRSS2 sequences (e.g., coding exons 4-13 of human TMPRSS2) and confirmed the loss and/or retention of mouse Tmprss2 sequences (e.g., loss of coding exons 4-13 of mouse Tmprss2). Table 1 sets forth the primers and probes that were used to confirm humanization of an endogenous Tmprss2 gene as described above (FIGS. 1A-1B). Once a correctly targeted ES cell clone has been selected, the neomycin selection cassette can be excised by introducing a Cre recombinase, e.g., via electroporation. Alternatively, the neomycin selection cassette can be removed by crossing the progeny generated from the ES clone with a deletor rodent strain that expresses a Cre recombinase. The humanized Tmprss2 locus after the deletion of the cassette is depicted in FIG. 1C, with the junction sequences shown at the bottom of FIG. 1C.

Selected ES cell clones (with or without the cassette) were used to implant female mice using the VELOCIMOUSE® method (see, e.g., U.S. Pat. No. 7,294,754 and Poueymirou et al., F0 generation mice that are essentially fully derived from the donor gene-targeted ES cells allowing immediate phenotypic analyses, 2007, Nature Biotech. 25(1):91-99) to generate a litter of pups containing a humanized Tmprss2 allele in the genome. Mice bearing a humanized Tmprss2 allele can be again confirmed and identified by genotyping of DNA isolated from tail snips using a modification of allele assay (Valenzuela et al., supra) that detects the presence of the human TMPRSS2 gene sequences. Pups are genotyped and cohorts of animals heterozygous for the humanized Tmprss2 locus are selected for characterization. Animals homozygous for the humanized Tmprss2 locus are made by crossing heterozygous animals.

TABLE 1

| Name | Primer | Sequence (5′-3′) | SEQ ID NO |
|---|---|---|---|
| 7010U | Forward | GCCGTGACTGTGACCTTCTC | (SEQ ID NO: 26) |
| | Probe (BHQ) | TGGAGGAGCCACCTGATGCCTC | (SEQ ID NO: 27) |
| | Reverse | GCCTTGCCCTCAATGGAAAC | (SEQ ID NO: 28) |
| 7010D | Forward | GGTTGCACAGCAAGGAAGAAG | (SEQ ID NO: 29) |
| | Probe (BHQ) | CCAGGAGTTCCTGTGAGCCTACCC | (SEQ ID NO: 30) |
| | Reverse | TGGAATGGAAGGAGCTGGAG | (SEQ ID NO: 31) |
| 7010hU | Forward | GTCCCACCTCCTGCAACTG | (SEQ ID NO: 32) |
| | Probe (BHQ) | TGAGCCTTCCCATCAGCCTGGG | (SEQ ID NO: 33) |
| | Reverse | CCACAATGGCACATGGGTCTG | (SEQ ID NO: 34) |
| 7010hTD | Forward | GGTGCTTGCTCCCCAAGA | (SEQ ID NO: 35) |
| | Probe (BHQ) | CCTAAAAGGTGTTGTAATGG | (SEQ ID NO: 36) |
| | Reverse | GGCAATAAAGAAGGAAGACGTTTT | (SEQ ID NO: 37) |

### Example 2. Humanization of an Endogenous Tmprss4 Gene

This example illustrates exemplary methods of humanizing an endogenous gene encoding Tmprss4 in a rodent (e.g., a mouse). The methods described in this example can be employed to humanize an endogenous Tmprss4 gene of a rodent using any human sequence, or combination of human sequences (or sequence fragments) as desired.

A targeting vector for humanization of an endogenous Tmprss4 gene was constructed using bacterial artificial chromosome (BAC) clones and VELOCIGENE® technology (see, e.g., U.S. Pat. No. 6,586,251 and Valenzuela et al. (2003), supra).

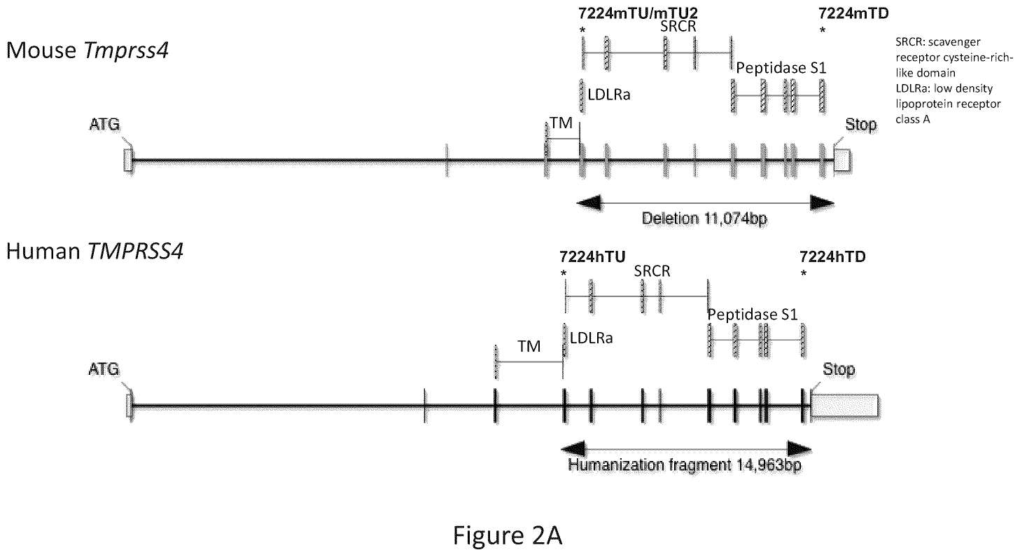

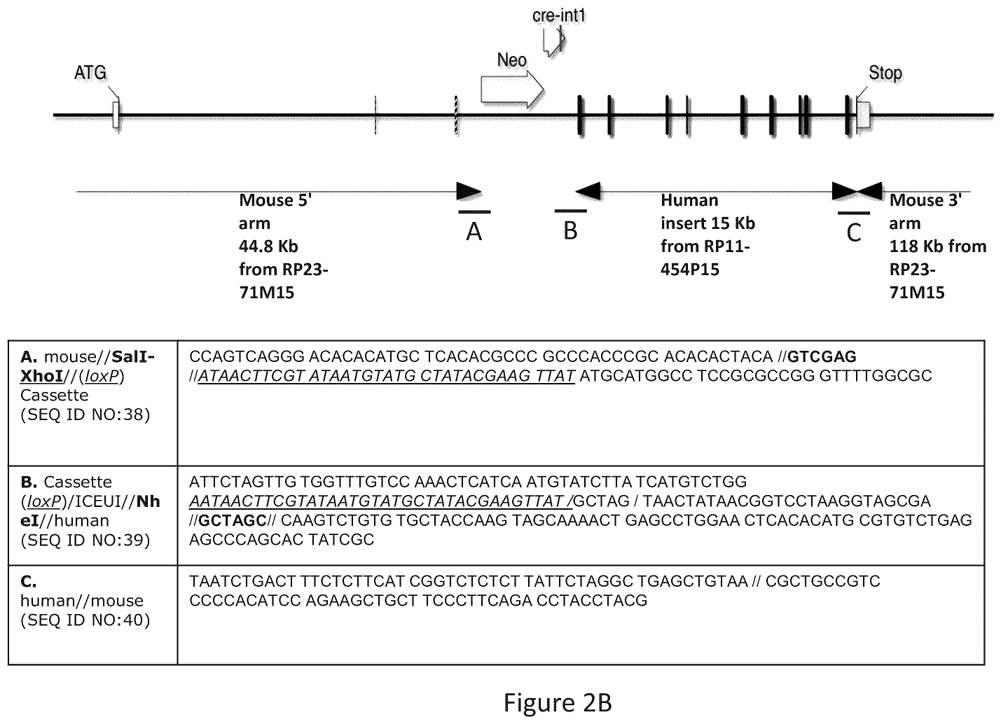

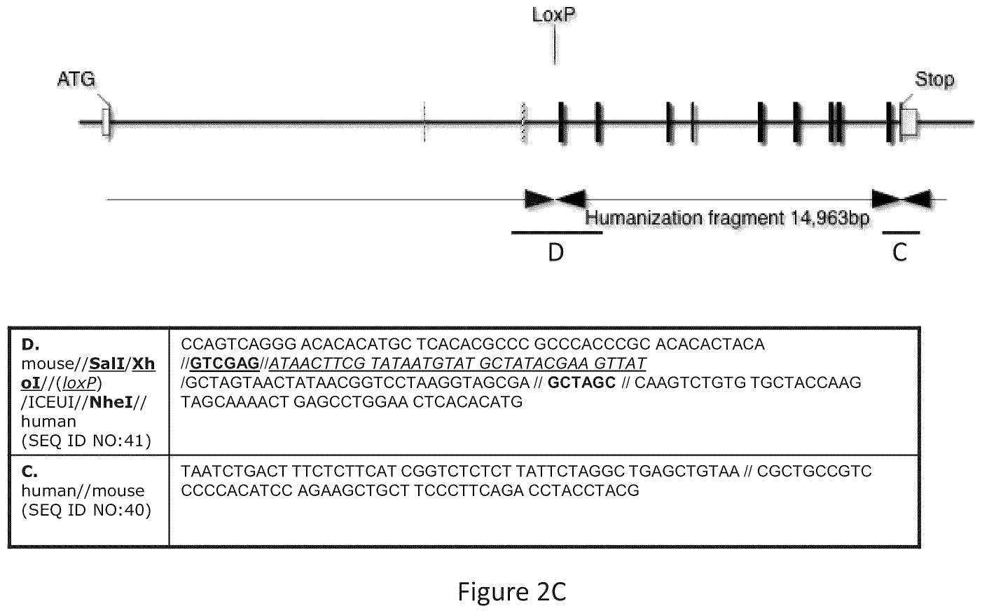

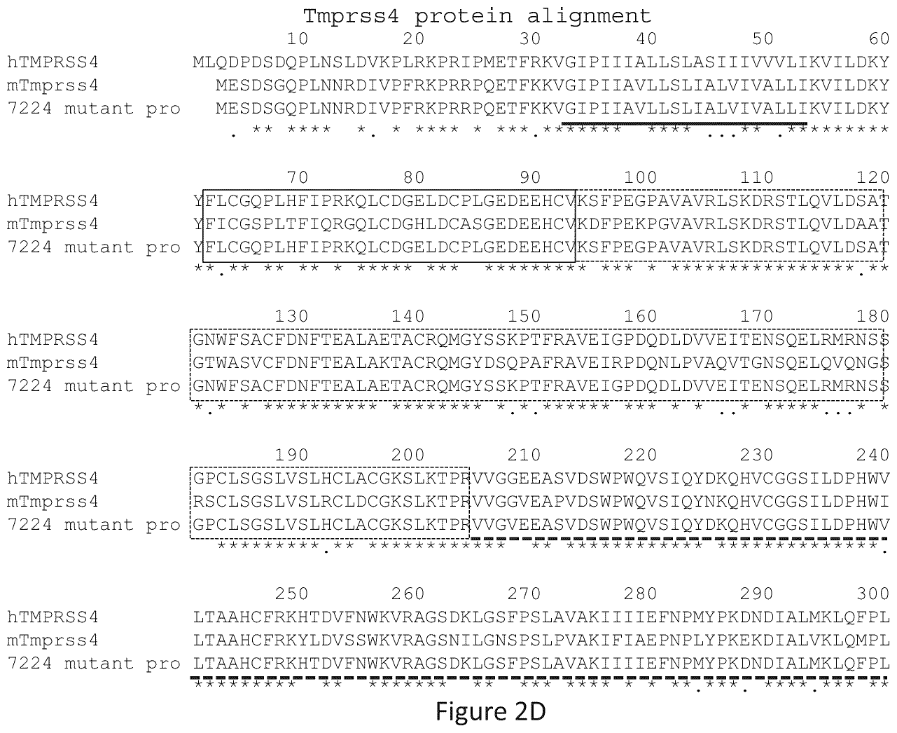

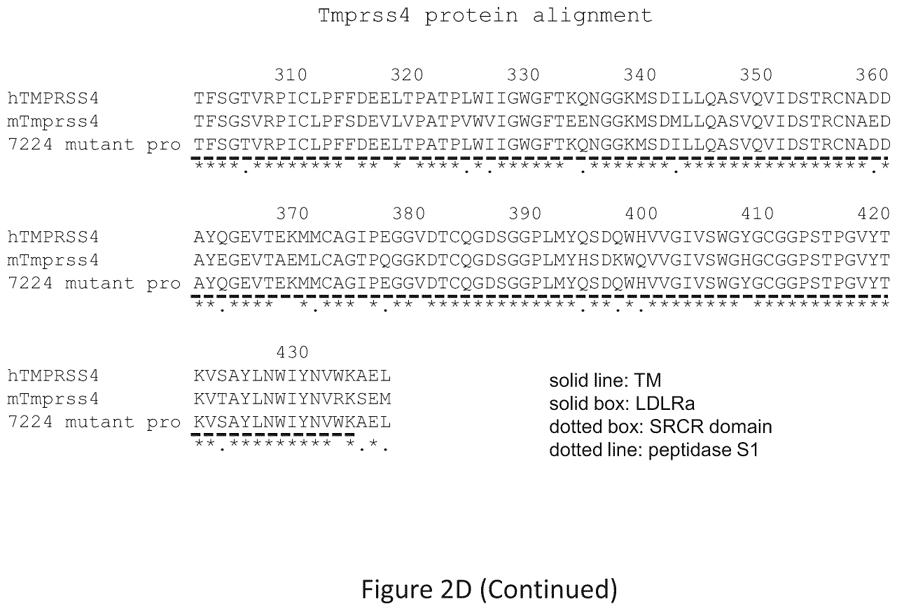

Briefly, mouse bacterial artificial chromosome (BAC) clone RP23-71M15 containing a mouse Tmprss4 gene was used and modified as follows. A DNA fragment was generated to include a 5′ mouse homology nucleotide sequence, a self-deleting neomycin cassette of about 4,996 bp, a human genomic DNA of about 14,963 bp (containing coding exon 4 through the stop codon in coding exon 13 of a human TMPRSS4 gene), and a 3′ mouse homology sequence. This DNA fragment was used to modify BAC clone RP23-71M15 through homologous recombination in bacterial cells. As a result, an ectodomain-encoding mouse genomic fragment (of about 11,074 bp) in the BAC clone was replaced with a self-deleting neomycin cassette of about 4,996 bp, followed by the human genomic DNA of about 14,963 bp. Specifically, the mouse genomic fragment that was deleted and replaced included the 3′ 130 bp of mouse intron 3, coding exon 4 through the stop codon in coding exon 13 of the mouse Tmprss4 gene (FIGS. 2A-2B). The human genomic fragment that was inserted included a 3′ portion of human TMPRSS4 intron 3 of about 150 bp, and human TMPRSS4 coding exon 4 through the stop codon in coding exon 13 (FIGS. 2A-2B). The resulting modified BAC clone included, from 5′ to 3′, a 5′ mouse homology arm containing about 44.8 kb of mouse genomic DNA (including a mouse Tmprss4 5′ UTR, mouse Tmprss4 coding exons 1 through 3, mouse Tmprss4 intron 3 in part (without the 3′ 130 bp), a self-deleting neomycin cassette of about 4996 bp, a 3′ portion of human TMPRSS4 intron 3 of about 150 bp, human TMPRSS4 coding exons 4 through the stop codon in coding exon 13, followed directly by the mouse Tmprss4 3′ UTR and the remaining mouse genomic DNA in the original BAC clone (a 3′ mouse homology arm of about 118 kb in total). See FIGS. 2A-2B. The junction sequences are also set forth at the bottom of FIG. 2B. The part of the modified BAC clone containing the neomycin cassette and the human TMPRSS4 genomic fragment, as well as the upstream and downstream insertion junctions, is set forth in SEQ ID NO: 12. The amino acid sequence of the protein encoded by the humanized Tmprss4 gene is set forth in SEQ ID NO: 14. An alignment of this humanized Tmprss4 protein (“7224 mutant pro”), a mouse Tmprss4 protein (SEQ ID NO: 9), and a human TMPRSS4 protein (SEQ ID NO: 11), is provided in FIG. 2D.

The modified BAC clone containing the humanized Tmprss4 gene, as described above, was used to electroporate mouse embryonic stem (ES) cells to create modified ES cells comprising a humanized Tmprss4 gene. Positively targeted ES cells containing a humanized Tmprss4 gene were identified by an assay (Valenzuela et al., supra) that detected the presence of the human TMPRSS4 sequences (e.g., coding exons 4-13 of human TMPRSS4) and confirmed the loss and/or retention of mouse Tmprss4 sequences (e.g., loss of coding exons 4-13 of mouse Tmprss4). Table 2 sets forth the primers and probes that were used to confirm humanization of an endogenous Tmprss4 gene as described above (FIGS. 2A-2B). Once a correctly targeted ES cell clone has been selected, the neomycin selection cassette can be excised by introducing a Cre recombinase, e.g., via electroporation. Alternatively, the neomycin selection cassette can be removed by crossing the progeny generated from the ES clone with a deletor rodent strain that expresses a Cre recombinase. The humanized Tmprss4 locus after the deletion of the cassette is depicted in FIG. 2C, with the junction sequences shown at the bottom of FIG. 2C.

Selected ES cell clones (with or without the cassette) were used to implant female mice using the VELOCIMOUSE® method (see, e.g., U.S. Pat. No. 7,294,754 and Poueymirou et al. (2007), supra) to generate a litter of pups containing a humanized Tmprss4 allele in the genome. Mice bearing a humanized Tmprss4 allele were again confirmed and identified by genotyping of DNA isolated from tail snips using a modification of allele assay (Valenzuela et al., supra) that detected the presence of the human TMPRSS4 gene sequences. Pups were genotyped and cohorts of animals heterozygous for the humanized Tmprss4 locus were selected for characterization. Animals homozygous for the humanized Tmprss4 locus were made by crossing heterozygous animals.

TABLE 2

| Name | Primer | Sequence (5′-3′) | SEQ ID NO |
|---|---|---|---|
| 7224mTU | Forward | GAGCAGGGCCATGACACAT | (SEQ ID NO: 42) |
| | Probe (BHQ) | ACCATTAGATCCCAGCACTGGACA | (SEQ ID NO: 43) |
| | Reverse | AAACCCTTCCCGAGAGAGAA | (SEQ ID NO: 44) |
| 7224mTU2 | Forward | GAGGAACACTGTGTCAAGGACTT | (SEQ ID NO: 45) |
| | Probe (BHQ) | CCTGAAAAGCCCGGAGTGGCAG | (SEQ ID NO: 46) |
| | Reverse | GGGCAGAGACCACATCTGA | (SEQ ID NO: 47) |
| 7224mTD | Forward | GGAAGCCCTCTCTCGATACTTG | (SEQ ID NO: 48) |
| | Probe (BHQ) | TTCTACCCTGAGGGCATGCAGC | (SEQ ID NO: 49) |
| | Reverse | TGGGATGTAGAAGGTTGTCAGA | (SEQ ID NO: 50) |
| 7224hTU | Forward | CTGAGCCTGGAACTCACACATG | (SEQ ID NO: 51) |
| | Probe (BHQ) | TCTGAGAGCCCAGCACTATCGCC | (SEQ ID NO: 52) |
| | Reverse | GCTGAGGGTCAGGCTTGAG | (SEQ ID NO: 53) |
| 7224hTD | Forward | TCTGCAGGGTAGGGAGAGAAG | (SEQ ID NO: 54) |
| | Probe (BHQ) | TGTTTCAGAAAAGGAAGACTCACG | (SEQ ID NO: 55) |
| | | TTACA | |
| | Reverse | GAGACCGATGAAGAGAAAGTCAGA | (SEQ ID NO: 56) |

### Example 3. Humanization of an Endogenous Tmprss11d Gene

This example illustrates exemplary methods of humanizing an endogenous gene encoding Tmprss11d in a rodent (e.g., a mouse). The methods described in this example can be employed to humanize an endogenous Tmprss11d gene of a rodent using any human sequence, or combination of human sequences (or sequence fragments) as desired.

A targeting vector for humanization of an endogenous Tmprss11d gene was constructed using bacterial artificial chromosome (BAC) clones and VELOCIGENE® technology (see, e.g., U.S. Pat. No. 6,586,251 and Valenzuela et al. (2003), supra).

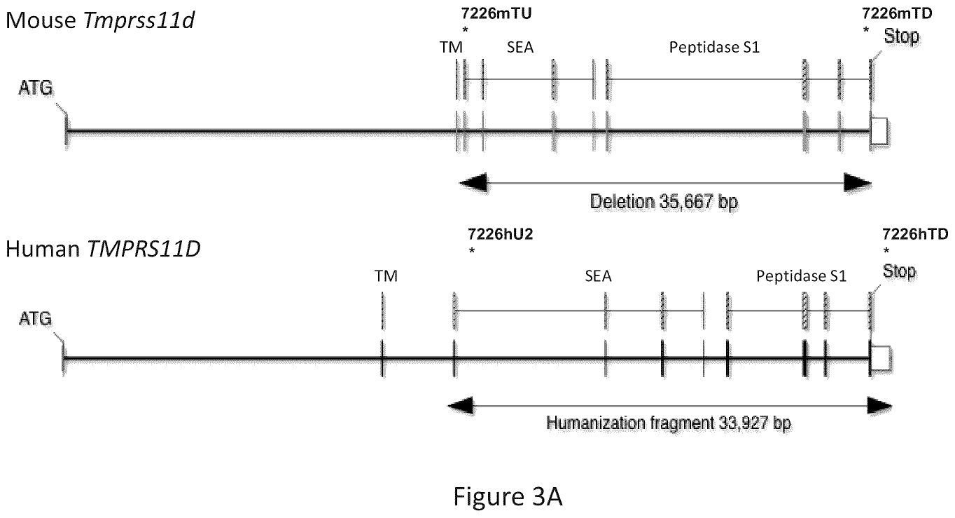

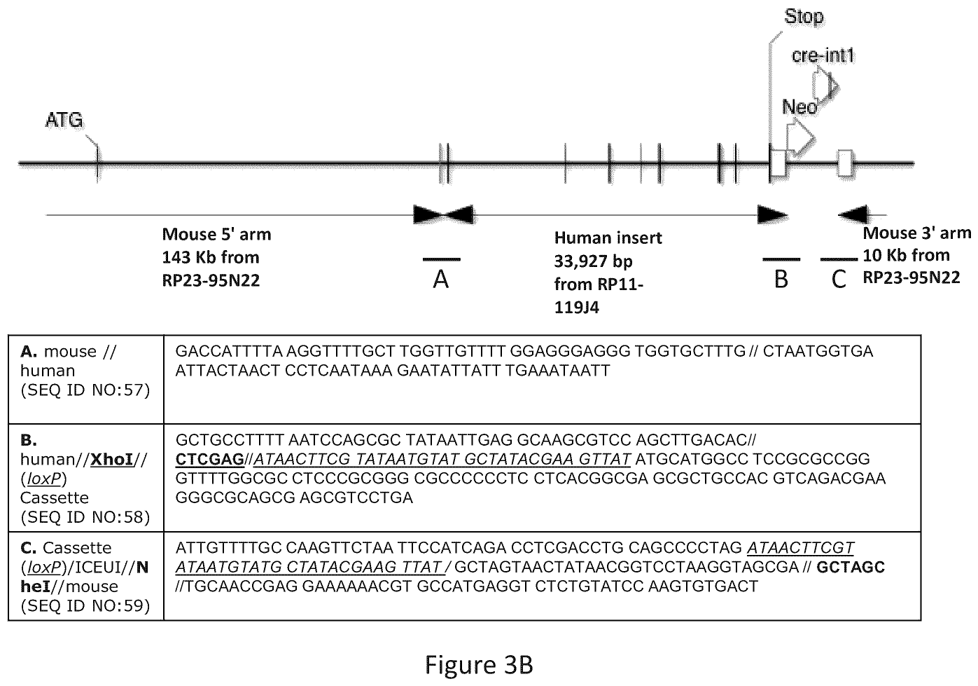

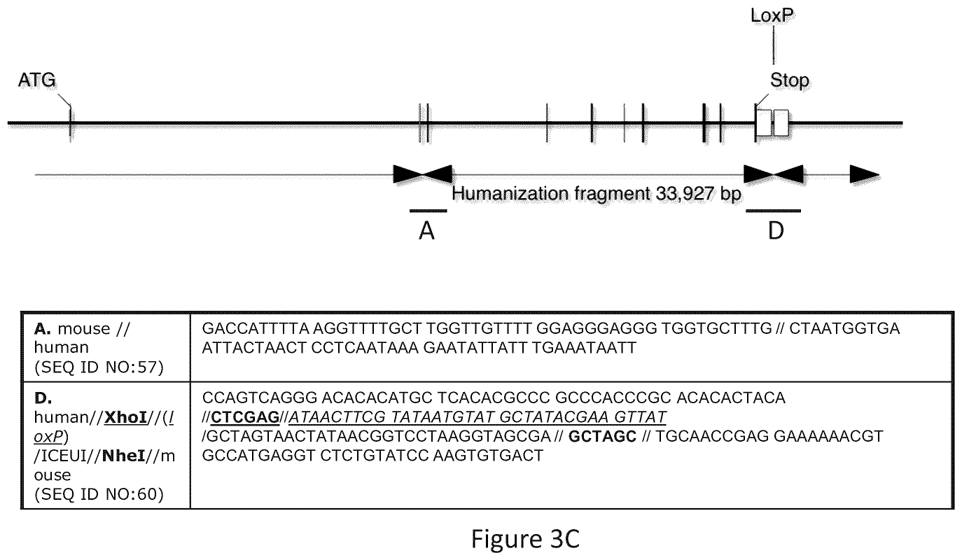

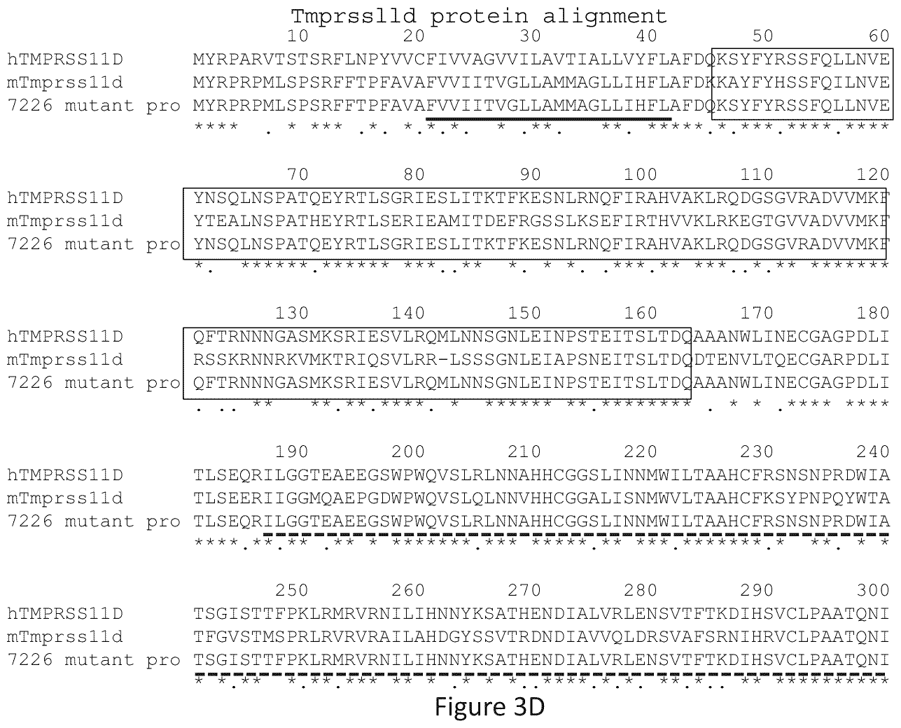

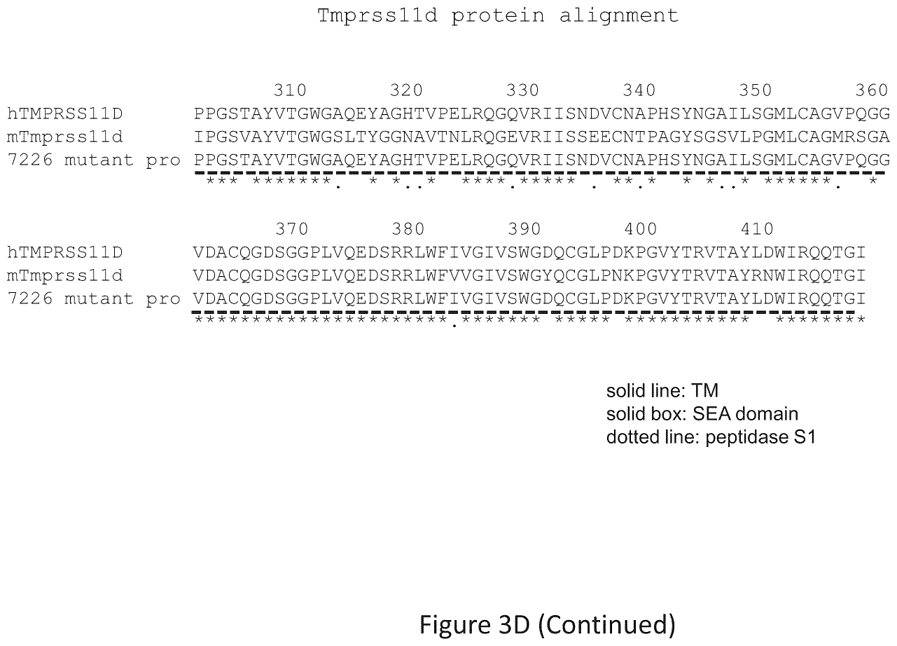

Briefly, mouse bacterial artificial chromosome (BAC) clone RP23-95N22 containing a mouse Tmprss11d gene was used and modified as follows. A DNA fragment was generated to include a 5′ mouse homology nucleotide sequence, a human TMPRSS11D genomic DNA of about 33,927 bp (containing 444 bp at the 3′ end of intron 2, and coding exon 3 through coding exon 10 (including the 3′ UTR which is part of coding exon 10), of a human TMPRSS11D gene), a self-deleting neomycin cassette of about 4,996 bp, and a 3′ mouse homology sequence. This DNA fragment was used to modify BAC clone RP23-95N22 through homologous recombination in bacterial cells. As a result, an ectodomain-encoding mouse Tmprss11d genomic fragment (of about 35,667 bp) in the BAC clone was replaced with the human TMPRSS11D genomic fragment of about 33,927 bp, followed by a self-deleting neomycin cassette of about 4,996 bp. Specifically, the mouse Tmprss11d genomic fragment that was replaced included a 3′ portion of intron 2, and coding exon 3 through the stop codon in coding exon 10 of the mouse Tmprss11d gene (FIGS. 3A-3B).

The human TMPRSS11D genomic fragment that was inserted included 444 bp at the 3′ end of intron 2, and coding exon 3 through coding exon 10 of a human TMPRSS11D gene (including the 3′ UTR of human TMPRSS11D), and a human 3′ genomic sequence of about 172 bp downstream of the 3′ UTR of human TMPRSS11D (FIGS. 3A-3B). The resulting modified BAC clone included, from 5′ to 3′, (i) a 5′ mouse homology arm containing about 143 kb of mouse genomic DNA including a mouse Tmprss11d 5′ UTR, mouse Tmprss11d coding exons 1-2 and a 5′ portion of intron 2; (ii) a human TMPRSS11D genomic fragment including a 3′ portion of intron 2 and coding exons 3 through 10 (including the 3′ UTR) of human TMPRSS11D, and a human 3′ genomic sequence; (iii) a self-deleting neomycin cassette of about 4,996 bp, followed by (iv) a 3′ mouse homology arm of 10 kb containing the mouse Tmprss11d 3′UTR and the remaining mouse genomic DNA in the original BAC clone. See FIGS. 3A-3B. The junction sequences are also set forth at the bottom of FIG. 3B. The part of the modified BAC clone containing the human TMPRSS11D genomic fragment and the neomycin cassette, as well as the upstream and downstream insertion junctions, is set forth in SEQ ID NO: 19. The amino acid sequence of the protein encoded by the humanized Tmprss11d gene is set forth in SEQ ID NO: 21. An alignment of this humanized Tmprss11d protein (“7226 mutant pro”), a mouse Tmprss11d protein (SEQ ID NO: 16), and a human TMPRSS 11D protein (SEQ ID NO: 18), is provided in FIG. 3D.

The modified BAC clone containing the humanized Tmprss11d gene, as described above, is used to electroporate mouse embryonic stem (ES) cells to create modified ES cells comprising a humanized Tmprss11d gene. Positively targeted ES cells containing a humanized Tmprss11d gene are identified by an assay (Valenzuela et al., supra) that detects the presence of the human TMPRSS11D sequences (e.g., coding exons 3-10 of human TMPRSS11D) and confirms the loss and/or retention of mouse Tmprss11d sequences (e.g., loss of coding exons 3-10 of mouse Tmprss11d). Table 3 sets forth the primers and probes that were used to confirm humanization of an endogenous Tmprss11d gene as described above (FIGS. 3A-3B). Once a correctly targeted ES cell clone has been selected, the neomycin selection cassette can be excised by introducing a Cre recombinase, e.g., via electroporation. Alternatively, the neomycin selection cassette can be removed by crossing the progeny generated from the ES clone with a deletor rodent strain that expresses a Cre recombinase. The humanized Tmprss11d locus after the deletion of the cassette is depicted in FIG. 3C, with the junction sequences shown at the bottom of FIG. 3C.

Selected ES cell clones (with or without the cassette) are used to implant female mice using the VELOCIMOUSE® method (see, e.g., U.S. Pat. No. 7,294,754 and Poueymirou et al. (2007), supra) to generate a litter of pups containing a humanized Tmprss11d allele in the genome. Mice bearing a humanized Tmprss11d allele are again confirmed and identified by genotyping of DNA isolated from tail snips using a modification of allele assay (Valenzuela et al., supra) that detects the presence of the human TMPRSS11D gene sequences. Pups are genotyped and cohorts of animals heterozygous for the humanized Tmprss11d locus are selected for characterization. Animals homozygous for the humanized Tmprss11d locus are made by crossing heterozygous animals.

TABLE 3

| Name | Primer | Sequence (5′-3′) | SEQ ID NO |
|---|---|---|---|
| 7226mTU | Forward | TCCTCTCCAGACAAGAAAGCT | (SEQ ID NO: 61) |
| | Probe (BHQ) | TCATAGCAGCTTTCAAATCCTAAACGT | (SEQ ID NO: 62) |
| | | TGA | |
| | Reverse | TCGTGTGTAGCTGGTGAGTT | (SEQ ID NO: 63) |
| 7226mTD | Forward | CATGCGATCACAGGAGGAGATC | (SEQ ID NO: 64) |
| | Probe (BHQ) | AATTGGGCCCGAAGCCAGATGC | (SEQ ID NO: 65) |
| | Reverse | CGGAAGGCTTCTGTGACTTC | (SEQ ID NO: 66) |
| 7226hTU | Forward | GTCTCCCACTTCTGACATAATGAAC | (SEQ ID NO: 67) |
| | Probe (BHQ) | CCCAGTGTTAACCCTACATCTGGTTCC | (SEQ ID NO: 68) |
| | Reverse | TGGGAAGAGACTCTTGGACA | (SEQ ID NO: 69) |
| 7226hTD | Forward | ATGAGCTCCTAGTACAGCTAAAGTT | (SEQ ID NO: 70) |
| | Probe (MGB) | ATGCATGATCATCTATGCGTCAGAGC | (SEQ ID NO: 71) |
| | Reverse | TGCCCAGATGCAGGGAGTTAG | (SEQ ID NO: 72) |

### Example 4. Evaluation of Group 1 and Group 2 Influenza a Viruses in MAID7225 HumIn Vs. Wild-Type Tmprss4 Mice

To validate the use of humanized Tmprss rodents as an animal model of infection, experiments were conducted to evaluate the survival of MAID7225 HumIn TMPRSS4 mice versus wild-type (WT) littermates in an influenza A group 1 and group 2 model of severe influenza infection.

MAID7225 HumIn TMPRSS4 mice are homozygous for a humanized Tmprss4 gene in its genome and were generated as described in Example 2. The viral strains used in these studies included the historical A/Puerto Rico/08/1934 (H1N1) influenza A virus group 1 isolate and an in-house mouse-adapted A/Aichi/02/1968 (HA, NA) X-31 (H3N2) influenza A virus group 2 isolate. All experiments were performed in 6-8 week-old male and female MAID7225 HumIn TMPRSS4 mice or WT littermates. Mice were challenged with 1150 plaque-forming units (PFUs) of A/Puerto Rico/08/1934 (H1N1) or 10,000 PFUs of A/Aichi/02/1968-X31 (H3N2). In these survival models, mice were challenged intranasally (IN) on day 0 post-infection (p.i.). Mice were weighed and observed daily up to day 14 p.i. and were sacrificed when they lost 20% of their starting weight. Results are reported as percent survival (Table 4).

TABLE 4

| | | Percent survival | |
|---|---|---|---|
| | Number of mice | (no. of surviving mice/total | |
| Group ID | per group | no. of mice in the group) | |
| Uninfected control (2 HumIn, 2 WT mice) | 4 | 100 | (4/4) |
| WT TMPRSS4; H1_PR34 infected | 10 | 20 | (2/10) |
| HumIn TMPRSS4; H1_PR34 infected | 8 | 25 | (2/8) |
| WT TMPRSS4; H3_X31 infected | 9 | 11.1 | (1/9) |
| HumIn TMPRSS4; H3_X31 infected | 8 | 25 | (2/8) |

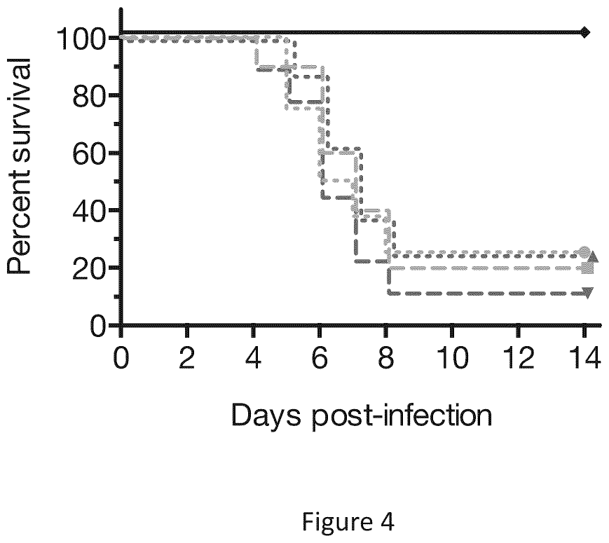

The survival of MAID7225 HumIn TMPRSS4 mice was compared to WT littermates after challenge with both severe Influenza A group 1 virus [A/Puerto Rico/08/1934 (H1N1)] and a severe, mouse-adapted Influenza A group 2 virus [A/Aichi/02/1968-X31 (H3N2)](FIG. 4). Survival of MAID7225 HumIn TMPRSS4 mice was no different from wild-type mice with either the H1N1 challenge (25%; n=8 and 20%; n=10, respectively) or the H3N2 challenge (25%; n=8 and 11.1%; n=9, respectively).

The publications, websites and other reference materials referenced herein to describe the background of the invention and to provide additional detail regarding its practice are hereby incorporated by reference.

What is claimed is:

1. A rodent comprising a humanized Tmprss gene that comprises a nucleotide sequence of an endogenous rodent Tmprss gene and a nucleotide sequence of a cognate human TMPRSS gene, wherein the humanized Tmprss gene is under control of the promoter of the endogenous rodent Tmprss gene.

2. The rodent of claim 1, wherein the humanized Tmprss gene encodes a humanized Tmprss protein that comprises an ectodomain substantially identical to the ectodomain of the human TMPRSS protein encoded by the cognate human TMPRSS gene.

3. The rodent of claim 2, wherein the humanized Tmprss protein further comprises a cytoplasmic and transmembrane portion that is substantially identical to the cytoplasmic and transmembrane portion of the endogenous rodent Tmprss protein encoded by the endogenous rodent Tmprss gene.

4. The rodent of claim 1, wherein the nucleotide sequence of the cognate human TMPRSS gene encodes a polypeptide substantially identical to the ectodomain of the human TMPRSS protein encoded by the cognate human TMPRSS gene.

5. The rodent of claim 1, wherein the nucleotide sequence of the endogenous rodent Tmprss gene encodes a polypeptide substantially identical to the cytoplasmic and transmembrane portion of the endogenous rodent Tmprss protein encoded by the endogenous rodent Tmprss gene.

6. The rodent of claim 1, wherein the humanized Tmprss gene is located at an endogenous rodent Tmprss locus, and results from a replacement of a genomic sequence of the endogenous rodent Tmprss gene with said nucleotide sequence of the cognate human TMPRSS gene.

7. The rodent of claim 1, wherein the humanized Tmprss gene is a humanized Tmprss2 gene, the endogenous rodent Tmprss gene is an endogenous rodent Tmprss2 gene, and the cognate human TMPRSS gene is a human TMPRSS2 gene.

8. The rodent of claim 7, wherein the humanized Tmprss2 gene encodes a humanized Tmprss2 protein that comprises an ectodomain substantially identical with the ectodomain of the human TMPRSS2 protein encoded by the human TMPRSS2 gene.

9. The rodent of claim 8, wherein the human TMPRSS2 protein comprises an amino acid sequence at least 85% identical with the amino acid sequence as set forth in SEQ ID NO: 4.

10. The rodent of claim 8, wherein the ectodomain of the humanized Tmprss2 protein comprises an amino acid sequence substantially identical with the amino acid sequence composed of residues W106 to G492 of SEQ ID NO: 4.

11. The rodent of claim 8, wherein the humanized Tmprss2 protein further comprises a cytoplasmic and transmembrane portion that is substantially identical with the cytoplasmic and transmembrane portion of the endogenous rodent Tmprss2 protein encoded by the endogenous rodent Tmprss2 gene.

12. The rodent of claim 7, wherein the nucleotide sequence of the human TMPRSS2 gene encodes a polypeptide that is substantially identical with the ectodomain of the human TMPRSS2 protein encoded by the human TMPRSS2 gene.

13. The rodent of claim 12, wherein the nucleotide sequence of the human TMPRSS2 gene comprises coding exon 4 through the stop codon in coding exon 13 of the human TMPRSS2 gene.

14. The rodent of claim 13, further comprising the 3′ UTR of the human TMPRSS2 gene.

15. The rodent of claim 7, wherein the nucleotide sequence of the endogenous rodent Tmprss2 gene encodes a cytoplasmic and transmembrane portion that is substantially identical with the cytoplasmic and transmembrane portion of the endogenous rodent Tmprss2 protein encoded by the endogenous rodent Tmprss2 gene.

16. The rodent of claim 7, wherein the humanized Tmprss2 gene comprises coding exons 1-2 of the endogenous rodent Tmprss2 gene, and coding exon 4 through coding exon 13 of the human TMPRSS2 gene, wherein the humanized Tmprss2 gene encodes a humanized Tmprss2 protein that comprises a cytoplasmic and transmembrane portion that is substantially identical with the cytoplasmic and transmembrane portion of the rodent Tmprss2 protein encoded by said endogenous rodent Tmprss2 gene, and an ectodomain that is substantially identical with the ectodomain of the human TMPRSS2 protein encoded by said human TMPRSS2 gene.

17. The rodent of claim 16, wherein the humanized Tmprss2 gene comprises an exon 3 that comprises a 5′ portion of coding exon 3 of the endogenous rodent Tmprss2 gene and a 3′ portion of coding exon 3 of the human TMPRSS2 gene.

18. The rodent of claim 1, wherein the humanized Tmprss gene is a humanized Tmprss4 gene, the endogenous rodent Tmprss gene is an endogenous rodent Tmprss4 gene, and the cognate human TMPRSS gene is a human TMPRSS4 gene.

19. The rodent of claim 18, wherein the humanized Tmprss4 gene encodes a humanized Tmprss4 protein that comprises an ectodomain that is substantially identical with the ectodomain of the human TMPRSS4 protein encoded by the human TMPRSS4 gene.

20. The rodent of claim 19, wherein the human TMPRSS4 protein comprises an amino acid sequence at least 85% identical with the amino acid sequence as set forth in SEQ ID NO: 11.

21. The rodent of claim 19, wherein the ectodomain comprises an amino acid sequence substantially identical with the amino acid sequence composed of residues K54-L437 of SEQ ID NO: 11.

22. The rodent of claim 19, wherein the humanized Tmprss4 protein further comprises a cytoplasmic and transmembrane portion that is substantially identical with the cytoplasmic and transmembrane portion of the endogenous rodent Tmprss4 protein encoded by the endogenous rodent Tmprss4 gene.

23. The rodent of claim 18, wherein the nucleotide sequence of the human TMPRSS4 gene encodes an ectodomain substantially identical with the ectodomain of the human TMPRSS4 protein encoded by the human TMPRSS4 gene.

24. The rodent of claim 23, wherein the nucleotide sequence of the human TMPRSS4 gene comprises coding exon 4 through the stop codon in coding exon 13 of the human TMPRSS4 gene.

25. The rodent of claim 24, wherein the stop codon of the human TMPRSS4 gene is followed by the 3′ UTR of the endogenous rodent Tmprss4 gene.

26. The rodent of claim 18, wherein the nucleotide sequence of the endogenous rodent Tmprss4 gene encodes a cytoplasmic and transmembrane portion that is substantially identical with the cytoplasmic and transmembrane portion of the endogenous rodent Tmprss4 protein encoded by the endogenous rodent Tmprss4 gene.

27. The rodent of claim 18, wherein the humanized Tmprss4 gene comprises coding exon 1 through coding exon 3 of the endogenous rodent Tmprss4 gene, and coding exon 4 through the stop codon in coding exon 13 of the human TMPRSS4 gene.

28. The rodent of claim 1, wherein the humanized Tmprss gene is a humanized Tmprss11d gene, the endogenous rodent Tmprss gene is an endogenous rodent Tmprss11d gene, and the cognate human TMPRSS gene is a human TMPRSS11D gene.

29. The rodent of claim 28, wherein the humanized Tmprss11d gene encodes a humanized Tmprss11d protein that comprises an ectodomain that is substantially identical with the ectodomain of the human TMPRSS11D protein encoded by the human TMPRSS11D gene.

30. The rodent of claim 29, wherein the human TMPRSS11D protein comprises an amino acid sequence at least 85% identical with the amino acid sequence as set forth in SEQ ID NO: 18.

31. The rodent of claim 29, wherein the ectodomain comprises an amino acid sequence substantially identical with the amino acid sequence composed of residues A42 to 1418 of SEQ ID NO: 18.

32. The rodent of claim 29, wherein the humanized Tmprss11d protein further comprises a cytoplasmic and transmembrane portion that is substantially identical with the cytoplasmic and transmembrane portion of the endogenous rodent Tmprss11d protein encoded by the endogenous rodent Tmprss11d gene.

33. The rodent of claim 28, wherein the nucleotide sequence of the human TMPRSS11D gene encodes an ectodomain that is substantially identical with the ectodomain of the human TMPRSS11D protein encoded by the human TMPRSS11D gene.

34. The rodent of claim 33, wherein the nucleotide sequence of the human TMPRSS11D gene comprises coding exon 3 through the stop codon in coding exon 10 of the human TMPRSS11D gene.

35. The rodent of claim 34, further comprising the 3′ UTR of the human TMPRSS11D gene.

36. The rodent of claim 28, wherein the nucleotide sequence of the endogenous rodent Tmprss11d gene encodes a cytoplasmic and transmembrane portion that is substantially identical with the cytoplasmic and transmembrane portion of the endogenous rodent Tmprss11d protein encoded by endogenous rodent Tmprss11d gene.

37. The rodent of claim 28, wherein the humanized Tmprss11d gene comprises coding exons 1-2 of the endogenous rodent Tmprss11d gene, and coding exons 3 through coding exon 10 of the human TMPRSS11D gene.

38. The rodent of claim 1, wherein the rodent is a mouse or a rat.

39. The rodent of claim 1, wherein the rodent is heterozygous for the humanized Tmprss gene.

40. The rodent of claim 1, wherein the rodent is homozygous for the humanized Tmprss gene.

41. The rodent of claim 1, wherein the rodent comprises at least two humanized Tmprss genes at cognate endogenous Tmprss gene loci.

42. An isolated rodent cell or tissue whose genome comprises a humanized Tmprss gene that comprises a nucleotide sequence of an endogenous rodent Tmprss gene and a nucleotide sequence of a cognate human TMPRSS gene, wherein the humanized Tmprss gene is under control of the promoter of the endogenous rodent Tmprss gene.

43. The isolated rodent cell or tissue of claim 42, wherein the humanized Tmprss gene is selected from the group consisting of a humanized Tmprss2 gene, a humanized Tmprss4 gene, and a humanized Tmprss11d gene.

44. A rodent embryonic stem cell whose genome comprises a humanized Tmprss gene that comprises a nucleotide sequence of an endogenous rodent Tmprss gene and a nucleotide sequence of a cognate human TMPRSS gene, wherein the humanized Tmprss gene is under control of the promoter of the endogenous rodent Tmprss gene.

45. The rodent embryonic stem cell of claim 44, wherein the humanized Tmprss gene is selected from the group consisting of a humanized Tmprss2 gene, a humanized Tmprss4 gene, and a humanized Tmprss11d gene.

46. A rodent embryo generated from the rodent embryonic stem cell of claim 44.

47. A vector comprising a human genomic DNA encoding the ectodomain of a human TMPRSS protein, flanked by a 5′ nucleotide sequence and a 3′ nucleotide sequence that are homologous to genomic DNA sequences flanking a rodent genomic DNA at a rodent Tmprss locus encoding the ectodomain of a cognate rodent Tmprss protein.

48. A method of providing a rodent whose genome comprises a humanized Tmprss gene, the method comprising:
modifying the genome of a rodent to replace a genomic sequence of an endogenous Tmprss gene with a genomic sequence of a cognate human TMPRSS gene to form a humanized Tmprss gene.

49. A method of making a rodent having a humanized Tmprss gene, comprising:
(a) inserting a genomic fragment into an endogenous rodent Tmprss locus in a rodent embryonic stem cell, said genomic fragment comprising a nucleotide sequence of a cognate human TMPRSS gene, thereby forming a humanized Tmprss gene, wherein the humanized Tmprss gene is under control of the promoter of the rodent Tmprss gene at the endogenous rodent Tmprss locus;
(b) obtaining a rodent embryonic stem cell comprising the humanized Tmprss gene of (a); and,
(c) creating a rodent using the rodent embryonic stem cell of (b).

50. The method of claim 49, wherein the humanized Tmprss gene encodes a humanized Tmprss protein that comprises an ectodomain substantially identical to the ectodomain of the human TMPRSS protein encoded by the cognate human TMPRSS gene.

51. The method of claim 50, wherein the humanized Tmprss protein further comprises a cytoplasmic and transmembrane portion that is substantially identical to the cytoplasmic and transmembrane portion of the rodent Tmprss protein encoded by the rodent Tmprss gene at said endogenous rodent Tmprss locus.

52. The method of claim 49, wherein the humanized Tmprss gene is selected from the group consisting of a humanized Tmprss2 gene, a humanized Tmprss4 gene, and a humanized Tmprss11d gene.

53. The method of claim 52, wherein the humanized Tmprss2 gene comprises coding exons 1-2 of the endogenous rodent Tmprss2 gene, and coding exon 4 through coding exon 13 of the human TMPRSS2 gene.

54. The method of claim 53, wherein the humanized Tmprss2 gene comprises an exon 3 that comprises a 5′ portion of coding exon 3 of the endogenous rodent Tmprss2 gene and a 3′ portion of coding exon 3 of the human TMPRSS2 gene.

55. The method of claim 52, wherein the humanized Tmprss4 gene comprises coding exon 1 through coding exon 3 of the endogenous rodent Tmprss4 gene, and coding exon 4 through the stop codon in coding exon 13 of the human TMPRSS4 gene.

56. The method of claim 52, wherein the humanized Tmprss11d gene comprises coding exons 1-2 of the endogenous rodent Tmprss11d gene, and coding exons 3 through coding exon 10 of the human TMPRSS11D gene.

57. The method of claim 52, wherein the humanized Tmprss gene encodes a humanized Tmprss protein that comprises the ectodomain of a human TMPRSS protein selected from the group consisting of a human TMPRSS2 protein, a human TMPRSS4 protein, and a human TMPRSS11D protein.

58. The method of claim 57, wherein the humanized Tmprss protein is a humanized Tmprss2 protein comprising W106 to G492 or the C-terminal 387 amino acids of a human TMPRSS2 protein.

59. The method of claim 57, wherein the humanized Tmprss protein is a humanized Tmprss4 protein comprising K54-L437 or the C-terminal 384 amino acids of a human TMPRSS4 protein.

60. The method of claim 57, wherein the humanized Tmprss protein is a humanized Tmprss11d protein comprising A42-I418 or the C-terminal 377 amino acids of a human TMPRSS11D protein.

61. The method of claim 49, wherein the rodent is a mouse or a rat.

62. A method of assessing the therapeutic efficacy of a compound in treating influenza virus infection, comprising:
providing a rodent according to any one of claims 1-41;
administering an influenza virus and a candidate compound to the rodent; and
monitoring the presence and severity of influenza virus infection in the rodent to determine the therapeutic efficacy of the candidate compound.

63. The method of claim 62, wherein the influenza virus is administered to the rodent before the candidate compound.

64. The method of claim 62, wherein the influenza virus is administered to the rodent after the candidate compound.

65. The method of claim 62, wherein the candidate compound is an antibody or antigen-binding fragment thereof specific for a human TMPRSS protein.

66. The method of claim 65, wherein the human TMPRSS protein is selected from the group consisting of a human TMPRSS2 protein, a human TMPRSS4 protein, and a human TMPRSS11D protein.

67. The method of claim 62, wherein the rodent is a mouse or a rat.
